(19) United States
(12) Patent Application Publication
(10) Pub. No.: US 2012/0232062 A1
(43) Pub. Date: Sep. 13, 2012

(54) AZAINDAZOLES TO TREAT FLAVIVIRIDAE VIRUS INFECTION

(75) Inventors: Wenjin Yang, Foster City, CA (US); Ingrid Choong, Los Altos, CA (US)

(73) Assignee: EIGER BIOPHARMACEUTICALS, INC., San Carlos, CA (US)

(21) Appl. No.: 13/501,733

(22) PCT Filed: Oct. 19, 2010
(86) PCT No.: PCT/US10/53255

§ 371 Date: May 14, 2012

Related U.S. Application Data
(60) Provisional application No. 61/253,296, filed on Oct. 20, 2009.
(60) Provisional application No. 61/295,612, filed on Jan. 15, 2010.
(60) Provisional application No. 61/313,641, filed on Mar. 12, 2010.
(60) Provisional application No. 61/382,874, filed on Sep. 14, 2010.
(60) Provisional application No. 61/382,853, filed on Sep. 14, 2010.

(51) Int. Cl. A61K 31/437; A61P 31/12; C12N 5/02; C07D 471/04; A61K 31/55

(52) U.S. Cl. 514/217.07; 540/597; 540/481; 514/303; 435/375

(57) ABSTRACT

Azaindazole compounds are useful for treating Flaviviridae virus infection, including HCV infection.

### CROSS-REFERENCE TO RELATED APPLICATIONS

This application claims priority to U.S. Provisional Patent Application Ser. Nos. 61/253,296 filed on Oct. 20, 2009, 61/295,612 filed on Jan. 15, 2010, 61/313,641 filed on Mar. 12, 2010, 61/382,853 filed on Sep. 14, 2010, and 61/382,874, filed on Sep. 14, 2010, each of which is incorporated herein by reference in its entirety.

### FIELD OF THE INVENTION

The present invention provides compositions and methods for treating viral infection and so relates to the fields of biology, chemistry, medicinal chemistry, medicine, molecular biology, and pharmacology.

### BACKGROUND OF THE INVENTION

Over 150 million people are infected with Hepatitis C Virus (HCV) worldwide. Unfortunately, many of these individuals are unable to clear their infection with the current standard of care, which consists of treatment with a combination of interferon and ribavirin. Moreover, this treatment is associated with significant side effects, precluding its use by many individuals. Thus, current therapies are inadequate for the majority of the patients, and there is a pressing need for new drugs to treat HCV infection (See, Annals Internal Med. 132:296-305 (2000)).

The 9.6-kb positive single-stranded RNA HCV genome encodes a 3,000-amino-acid polyprotein which is proteolytically processed into structural proteins, which are components of the mature virus, and nonstructural proteins (NS), which are involved in replicating the viral genome (Curr Top Microbiol Immunol 242, 55-84 (2000)). Like other positive strand RNA viruses (Fields et al. (Eds.), Fields Virology. (Lippincott-Raven Publications, Philadelphia, Pa., 1996, in “The viruses and their replication”)), HCV appears to replicate in association with intracellular membrane structures. In the case of HCV, the structures are termed the membranous web (J Virol 76, 5974-5984 (2002)) and are believed to be induced by the NS4B protein. NS4B is also required to assemble the other viral NS proteins within the apparent sites of RNA replication (J Virol 78, 11393-11400 (2004)).

While promising new therapies for HCV infection based on compounds that inhibit the function of HCV proteins such as NS4B are in development (see, e.g., PCT Pub. Nos. 2009/038248; 2010/107739; and 2010/107742, each of which is incorporated herein by reference), there remains a need for new therapies based on more active compounds and/or compounds with fewer, or less severe side effects. The present invention meets that need.

### SUMMARY OF THE INVENTION

In one aspect, the present invention provides a compound of Formula I:

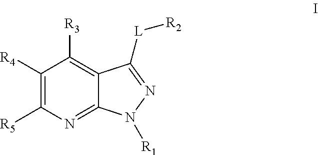

or a pharmaceutically acceptable salt thereof, wherein

R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;

L is a bond, —CONH—, —NH—CO—, substituted or unsubstituted C₁-C₅ alkylene, substituted or unsubstituted C₂-C₅ heteroalkylene, a substituted or unsubstituted 5 membered heteroaryl group, or a combination thereof;

R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group;

R₃, R₄, and R₅ are independently hydrogen, halo, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, —N(R′)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and

R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring.

In another aspect, the present invention provides a pharmaceutical composition comprising, consisting essentially of, or consisting of a compound of the present invention and a pharmaceutically acceptable carrier, diluent, and/or excipient. The compounds and compositions of the invention are useful in treating a Flaviviridae virus infection, including but not limiting to infection with HCV. Thus, in another aspect, the invention provides methods for treating a Flaviviridae virus infection, including methods for treating HCV infection. In various embodiments, the virus is HCV, including any of the various genotypes, such as, without limitation, genotypes 4, 2a, 1b, and 1a. The pharmaceutically acceptable compositions of the invention are useful in these methods. In such treatment methods, the virus or a cell infected with the virus, including but not limited to HCV, is contacted with a compound of the invention and replication of the virus is reduced or inhibited. The contacting can be in vitro or in vivo. In certain embodiments, the cell is a liver cell. When in vitro, the method can be used as a comparative for testing the activity of other antiviral compounds or testing the efficacy of combination therapies. When practiced in vivo in a patient other than a human patient, the method can serve as an animal model for pre-clinical studies or as a comparative similar to the in vitro use. In certain embodiments, particularly embodiments in which a human patient is administered a composition of the invention, a viral infection, such as an HCV infection, is treated by administering a therapeutically effective amount of a compound or pharmaceutical composition of the present invention to a patient in need of such treatment, e.g., a patient infected with the virus.

In another aspect, the present invention provides methods for making the compounds and compositions of the invention.

### DETAILED DESCRIPTION OF THE INVENTION

The detailed description is divided into various sections only for the reader's convenience and disclosure found in any section may be combined with that in another section. In Section I, definitions of terms used herein are provided. In Section II, various compounds useful in the methods of the invention are described. In Section III, infections amenable to treatment in accordance with the methods of the invention are described. In Section IV, pharmaceutical compositions, unit dose forms, and methods for administering the compounds, pharmaceutical compositions, and unit dose forms useful in accordance with the methods of the invention are described. In Section V, combination therapies of the invention are described. Section VI is followed by examples illustrating how the anti-viral activity of various illustrative compounds useful in the methods of the invention can be measured.

### I. Definitions

The terminology used herein is for the purpose of describing particular embodiments only, and is not intended to be limiting, because the scope of the present invention will be limited only by the appended claims. The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, should be viewed as approximations which may be varied (+) or (−), as appropriate, by increments of, for example, 0.1 or 1.0, for example. While not always explicitly stated, all numerical designations should be read as preceded by the term “about”. The reagents described herein should be viewed as exemplary, because equivalents of most reagents are known in the art. All technical and patent publications cited herein are incorporated herein by reference in their entirety. Nothing herein is to be construed as an admission that the invention is not entitled to antedate such disclosure by virtue of prior invention.

The singular forms “a,” “an,” and “the” include plural referents unless the context clearly dictates otherwise. Thus, for example, reference to “a compound” includes a plurality of compounds.

The term “administration” refers to introducing an agent into a host. Preferred routes of administration of the agents include oral administration and intravenous administration. An effective amount is administered, which amount can be determined by the treating physician or the like. Any route of administration, such as topical, subcutaneous, peritoneal, intraarterial, inhalation, vaginal, rectal, nasal, introduction into the cerebrospinal fluid, or instillation into body compartments can be used. The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

The terms “alkyl” refer to straight or branched chain hydrocarbon groups having 1 to 12 (or more as specified) carbon atoms, including, but not limited to, groups selected from 1 to 8 carbon atoms, such as methyl, ethyl, n-propyl, i-propyl, n-butyl, i-butyl, t-butyl, pentyl, hexyl, heptyl, n-octyl, dodecyl, octadecyl, amyl, 2-ethylhexyl, and the like. “C₁-C₆ alkyl” refers to a substituted or unsubstituted straight or branched chain alkyl groups having 1-6 carbon atoms. The term “substituted alkyl” refers to alkyl groups substituted with one or more groups, including, but not limited to, groups selected from alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, and the like.

The term acyl refers to “alkanoyl,” “substituted alkanoyl,” “aroyl,” or “substituted aroyl.”

The term “aryloxy” refers to —O-aryl, and the term “substituted aryloxy” refers to —O-substituted aryl.

The term “alkylester” refers to —O—CO-alkyl, and the term “substituted alkylester” refers to —O—CO-substituted alkyl.

The term “arylester” refers to —O—CO-aryl, and the term “substituted arylester” refers to —O—CO-substituted aryl.

The term “alkanoyl” refers to an alkyl group or a substituted alkyl group linked to a carbonyl group (i.e. —C(O)-alkyl or —C(O)-substituted alkyl). Similarly, the term “aroyl” refers to an aryl group or a substituted aryl group linked to a carbonyl group (i.e., —C(O)-aryl or —C(O)-substituted aryl).

The term “alkenyl” refers to straight or branched chain hydrocarbon groups having 2 to 12 (or more as specified) carbon atoms, including, but not limited to, groups having 2 to 4 carbon atoms, and at least one double carbon to carbon bond (either cis or trans), such as ethenyl. Alkenyl groups may be mono or polyunsaturated. Examples include, but are not limited to, vinyl, —CH═C(H)(CH₃), —CH═C(CH₃)₂, —C(CH₃)═CH₂, —C(CH₃)═C(H)(CH₃), C(CH₂CH₃)═CH₂, and butadienyl. The term “substituted alkenyl” refers to alkenyl groups substituted with one or more groups, including, but not limited to, groups selected from alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, and the like.

The term “alkoxy” refers to O-alkyl. For example, the methoxy group CH₃O— is an alkoxy group. “C₁-C₆ alkoxy” refers to a substituted or unsubstituted alkyl group of 1-6 carbon atoms covalently bonded to an oxygen atom. In other words, a C₁-C₆ alkoxy group has the general structure —O—(C₁-C₆) alkyl. C₁-C₆ alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy. The term “substituted alkoxy” refers to —O-substituted alkyl.

The term “alkoxyamino” refers to —NH-alkoxy. The term “substituted alkoxyamino” refers to —NH-substituted alkoxy.

The term “alkylene” refers to a linear saturated divalent hydrocarbon radical or a branched saturated divalent hydrocarbon radical. Similarly, C₁-C₁₀ alkylene refers to a corresponding alkylene group having 1-10 carbon atoms. C₁-C₆ alkylene groups include, for example, without limitation, methylene, ethylene, propylene, butylene, 2-methylpropylene, and pentylene. The term “substituted alkylene” refers to alkyl groups substituted with one or more groups, including, but not limited to, groups selected from alkoxy (e.g., C₁ to C₇), substituted alkoxy, alkanoyl, substituted alkanoyl, alkenyl, substituted alkenyl, alkoxyamino, substituted alkoxyamino, alkylester, substituted alkylester, alkylthio, substituted alkylthio, alkynyl, substituted alkynyl, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, and the like.

The term “alkylthio” refers to −5-alkyl. The term “substituted alkylthio” refers to —S-substituted alkyl.

The term “alkynyl” refers to straight or branched chain hydrocarbon groups having 2 to 12 carbon atoms, including, but not limited to, groups having 2 to 4 carbon atoms, and at least one triple carbon to carbon bond, such as ethynyl. Alkynyl groups may be mono- or polyunsaturated, having the number of carbon atoms designated. Examples include, but are not limited to, ethynyl, 1-propynyl, —CC(CH₂CH₃), —C(H₂)CC(H), —C(H)₂CC(CH₃), and —C(H)₂CC(CH₂CH₃). The term “substituted alkynyl” refers to alkynyl groups substituted with one or more groups, including, but not limited to, groups selected from alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, and the like.

The term “amino” refers to a monovalent radical —NH₂. The term “alkylamino” refers to the group —NR^{a}R^{b} where R^{a} is alkyl or cycloalkyl and R^{b} is H. The term “dialkylamino” refers to the group —NR^{a}R^{b} where R^{a} and R^{b} independently are alkyl or cycloalkyl where the alkyl portions can be the same or different and can also be combined to form a 3- to 9-membered ring with the nitrogen atom to which each is attached. Accordingly, a dialylamino group represented as —NR^{a}R^{b} is meant to include piperidinyl, pyrrolidinyl, morpholinyl, azetidinyl, azepanyl and the like.

The term “aryl” refer to aromatic homocyclic (i.e., hydrocarbon) mono-, bi- or tricyclic ring-containing groups including, but not limited to, groups having 6 to 12 members such as phenyl, naphthyl and biphenyl. The term “substituted aryl” refers to aryl groups substituted with one or more groups, including, but not limited to, groups selected from alkyl, substituted alkyl, alkenyl, substituted alkenyl, alkynyl, substituted alkynyl, alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, and the like, where optionally one or more pair of substituents together with the atoms to which they are bonded form a 3 to 7 member ring.

The term “arylthio” refers to —S-aryl. The term “substituted arylthio” refers to —S-substituted aryl.

The term “carboxamide” or “amido” refers to —CON(R^{y})₂, wherein each R^{y} is independently hydrogen or is substituted or unsubstituted alkyl, alkenyl, alkynyl, cycloalkyl, heterocyclyl, aryl heteroaryl, acyl, sulfonyl, sulfonyloxy, or protected carboxy group, or two R^{y} groups together with the nitrogen atom to which they are bonded form a substituted or unsubstituted heterocycle or a heteroaryl group.

The term “carboxy” refers to —CO₂H.

The terms “(Cₘ-Cₙ)”, “Cₘ-Cₙ,” and “Cₘ₋ₙ” refer to the range (from “m” to “n”) in the number of carbon atoms in a certain group before which one of these terms is placed. For example, C₁-C₆ alkyl refers to an alkyl group containing from 1 to 6 carbon atoms.

The term “comprising” means that the compounds, compositions, and/or methods referenced in connection therewith include the recited elements following the term, but may or may not include (or exclude) other elements. The phrase “consisting essentially of” means that compounds, compositions and/or methods referenced in connection therewith include the recited elements following the term but exclude other elements that would materially affect the fundamental characteristics of the claimed invention. The phrase “consisting of” means that compounds, compositions and/or methods referenced in connection therewith include the recited elements following the term but exclude all other elements. Embodiments defined by each of these terms and phrases are provided by each of the different aspects of this invention.

The term “cycloalkyl” or “carbocyclo” refers to a mono-, bi-, or tricyclic saturated ring that is fully saturated or partially unsaturated. Thus, “cycloalkyl” refers to, unless otherwise stated, cyclic versions of “alkyl”, “alkenyl” and “alkynyl” in which all ring atoms are carbon. A cycloalkyl group may form a bridged ring or a spiro ring. The cycloalkyl group may have one or more double or triple bond(s). Typical cycloalkyl groups have from 3 to 9 ring atoms. Examples of such groups include cyclopropyl, cyclobutyl, cyclopentyl, cyclohexyl, 1-cyclohexenyl, 3-cyclohexenyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like. The term “substituted carbocyclo” refers to carbocyclo groups substituted with one or more groups, including, but not limited to, groups selected from substituted alkenyl, alkynyl, substituted alkynyl, alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, where optionally one or more pair of substituents together with the atoms to which they are bonded form a 3 to 7 member ring.

The term “(cycloalkyl)alkyl” refers to a cycloalkyl group substituted by an alkyl group. Examples include (cyclohexyl)methyl, 3-(cyclopropyl)-n-propyl, 5-(cyclopentyl)hexyl, 6-(adamantyl)hexyl, and the like.

The term “cycloalkyloxy” refers to —O-cycloalkyl. The term “substituted cycloalkyloxy” refers to —O-substituted cycloalkyl.

The term “cycloalkylthio” refers to —S-cycloalkyl. The term “substituted cycloalkylthio” refers to —S-substituted cycloalkyl.

The term “Flaviviridae virus” means any virus of the Flaviviridae family, including those viruses that infect humans and non-human animals. The polynucleotide and polypeptides sequences encoding these viruses are well known in the art, and may be found at NCBI's GenBank database, e.g., as Genbank Accession numbers NC_{—}004102, AB031663, D11355, D11168, AJ238800, NC_{—}001809, NC_{—}001437, NC_{—}004355, NC_{—}004119, NC_{—}003996, NC_{—}003690, NC_{—}003687, NC_{—}003675, NC_{—}003676, NC_{—}001563, NC_{—}000943, NC_{—}003679, NC_{—}003678, NC_{—}002657, NC_{—}002032, and NC_{—}001461, the contents of which database entries are incorporated by references herein in their entirety.

The term “guanidino” or guanidine refers to the group —NHC(═NH)NH₂. The term “substituted guanidino” refers to —NRC(═NR)N(R)₂, wherein each R is independently hydrogen, alkyl, substituted alkyl, aryl, substituted aryl, heteroaryl, substituted heteroaryl, heterocyclyl, or substituted heterocyclyl, or wherein two R groups attached to a common guanidino nitrogen atom may be joined together with the nitrogen bound thereto to form a heterocyclic or substituted heterocyclic group, provided that at least one R is not hydrogen.

The terms “halo” and “halogen” refer to the fluoro, chloro, bromo or iodo groups. There can be one or more halogen in a compound or attached to a moiety in a compound, which can be the same or different halo group(s).

The term “heteroalkylene” refers to an alkylene wherein 1-3 carbon atoms in the linear saturated divalent hydrocarbon radical or a branched saturated divalent hydrocarbon radical is replaced with a heteroatom. C₁-C₆ heteroalkylene groups include, for example, —O—CH₂—, —CH₂CH₂—O—CH₂CH₂—, —CH₂CH₂—NH—CH₂CH₂—, —CH₂—O—CH₂—, —CH₂—NH—CH₂— and —CH₂CH₂—S—CH₂CH₂—. The term “substituted heteroalkylene” refers to a heteroalkylene group substituted with one or more groups, including, but not limited to, groups selected from alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, and the like.

The term “heteroaryl” refers to optionally substituted aromatic rings that have 1 to 4 heteroatoms, such as oxygen, sulfur and/or nitrogen atoms. In particular, heteroaryl groups often contain nitrogen, either alone or in conjunction with sulfur or oxygen ring atoms, and often contain 1-3 five-membered or six-membered rings. Furthermore, the above optionally substituted five-membered or six-membered rings can optionally be fused to an aromatic 5-membered or 6-membered ring system. For example, the rings can be optionally fused to an aromatic 5-membered or 6-membered ring system, such as a pyridine or a triazole system or a benzene ring. Thus, heteroaryl can refer to a monocyclic aromatic system having 5 or 6 ring atoms, or to a fused ring bicyclic aromatic system having 8-20 atoms, in which the ring atoms are C, O, S, SO, SO₂, or N, and at least one of the ring atoms is a heteroatom, i.e., O, S, SO, SO₂, or N. The following ring systems are examples of radicals denoted by the term “heteroaryl”: acridinyl, azocinyl, benzimidazolyl, benzofuranyl, benzothio-furanyl, benzothiophenyl, benzoxazolyl, benzothiazolyl, benzotriazolyl, benzotetrazolyl, benzisoxazolyl, benzisothiazolyl, benzimidazolinyl, carbazolyl, NH-carbazolyl, carbolinyl, chromanyl, chromenyl, cinnolinyl, dithiazinyl, furanyl, furazanyl, imidazolidinyl, imidazolinyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazole, pyridinyl, pyridyl, pyrimidinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienyl, thienothiazolyl, thienooxazolyl, thienoimidazolyl, thiophenyl, triazinyl and xanthenyl. Unless indicated otherwise, the arrangement of the heteroatoms within the ring may be any arrangement allowed by the bonding characteristics of the constituent ring atoms. The term “substituted heteroaryl” refers to heteroaryl groups substituted with one or more groups, including, but not limited to, groups selected from substituted alkenyl, alkynyl, substituted alkynyl, alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, where optionally one or more pair of substituents together with the atoms to which they are bonded form a 3 to 7 member ring. In some embodiments, substituents for substituted heteroaryl rings can include from one to three acyl, halo, nitro, cyano, trihalomethyl, amino, protected amino, amido, amino salts, substituted amino, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, alkenyl, substituted alkenyl, alkynyl, substituted alkynyl, alkyl ester, aryl ester, phenyl, substituted phenyl, phenylalkyl, and (substituted phenyl)alkyl, sulfonyl (optionally substituted), sulfonyloxy (optionally substituted), and the like. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group; similarly, “lower alkylthio” means a C₁ to C₄ alkylthio group.

The term “heteroaryloxy” refers to —O-heteroaryl. The term “substituted heteroaryloxy” refers to —O-substituted heteroaryl.

The term “heteroarylthio” refers to —S-heteroaryl. The term “substituted heteroarylthio” refers to —S-substituted heteroaryl.

The term “heterocyclyl” “heterocyclic”, “heterocyclic group” or “heterocyclo” refers to a monocyclic or fused ring multicyclic cycloalkyl group in which one or more of the carbon atoms in the ring system is replaced by a heteroatom selected from O, S, SO, SO₂, P, or N, where the nitrogen and sulfur heteroatoms may optionally be oxidized and the nitrogen heteroatoms may optionally be quaternized. Heterocycle includes 3 to 13 member monocyclic, 7 to 17 member bicyclic, or 10 to 20 member tricyclic ring systems, typically containing a total of 3 to 10 ring atoms. Examples of heterocyclyl groups include but are not limited to azepanyl, imidazolinyl, morpholinyl, piperidinyl, piperidin-2-onyl, piperazinyl, pyrrolidinyl, pyrrolidine-2-onyl, tetrahydrofuranyl, and tetrahydroimidazo[4,5-c]pyridinyl. The terms “substituted heterocycle”, “substituted heterocyclic”, “substituted heterocyclic group” and “substituted heterocyclo” refer to heterocycle, heterocyclic and heterocyclo groups substituted with one or more groups, including, but not limited to, groups selected from alkenyl, substituted alkenyl, alkynyl, substituted alkynyl, alkoxy (e.g., C₁ to C₇), substituted alkoxy, alkanoyl, substituted alkanoyl, alkoxyamino, substituted alkoxyamino, alkylester, substituted alkylester, alkylthio, substituted alkylthio, amino, substituted amino, (monosubstituted)amino, (disubstituted)amino, protected amino, amido, arylthio, substituted arylthio, aryloxy (e.g., C₁ to C₇), substituted aryloxy, arylester, substituted arylester, aroyl, substituted aroyl, aryl, substituted aryl, azido, carbocyclo, substituted carbocyclo, carboxy, protected carboxy, cyano, cycloalkyloxy, substituted cycloalkyloxy, cycloalkylthio, substituted cycloalkylthio, halo, heteroaryloxy, substituted heteroaryloxy, heteroarylthio, substituted heteroarylthio, heterocyclo, substituted heterocyclo, heterocyclyloxy, substituted heterocyclyloxy, heterocyclylthio, substituted heterocyclylthio, hydroxy, protected hydroxy, hydroxyamino, hydrazino, substituted hydrazino, guanidino, substituted guanidino, lactam, nitro, oxo, sulfonyl, substituted sulfonyl, sulfonyloxy, substituted sulfonyloxy, —SO₃H, thioacyl, thiocyanate, thiol, thione, urea, urethane, where optionally one or more pair of substituents together with the atoms to which they are bonded form a 3 to 7 member ring Examples of heterocycle and heteroaryl groups include the following monocyclic, bicyclic, and tricyclic ring systems: pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, triazinyl, tetrahydropyranyl, tetrazoyl, triazolyl, morpholinyl, thiamorpholinyl, thiamorpholinyl sulfoxide, thiamorpholinyl sulfone, 1,3-dioxolane and tetrahydro-1,1-dioxothienyl, and the like. Exemplary such bicyclic groups include indolyl, benzothiazolyl, benzoxazolyl, benzothienyl, quinuclidinyl, quinolinyl, tetra-hydroisoquinolinyl, isoquinolinyl, benzimidazolyl, benzopyranyl, indolizinyl, benzofuryl, benzofuranly, dihydrobenzofuranyl, chromonyl, coumarinyl, benzodioxolyl, dihydrobenzodioxolyl, benzodioxinyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), tetrahydroquinolinyl, azabicycloalkyls (such as 6-azabicyclo[3.2.1]octane), azaspiroalkyls (such as 1,4 dioxa-8-azaspiro[4.5]decane), imidazopyridinyl (such as imidazo[1,5-a]pyridin-3-yl), triazolopyridinyl (such as 1,2,4-triazolo[4,3-a]pyridin-3-yl), and hexahydroimidazopyridinyl (such as 1,5,6,7,8,8a-hexahydroimidazo[1,5-a]pyridin-3-yl), and the like. Exemplary such tricyclic groups include carbazolyl, benzidolyl, phenanthrolinyl, acridinyl, phenanthridinyl, xanthenyl, and the like.

The term “heterocyclyloxy” refers to —O-heterocyclyl. The term “substituted heterocyclyloxy” refers to —O-substituted heterocyclyl.

The term “heterocyclylthio” refers to —S-heterocyclyl. The term “substituted heterocyclylthio” refers to —S-substituted heterocyclyl.

The terms “host,” “individual,” “subject,” “patient,” or “organism” include humans and mammals (e.g., mice, rats, pigs, cats, dogs, and horses). Typical hosts to which compounds of the present disclosure may be administered are mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living host” refers to any mammal or other animal listed above or any other organism that is alive. The term “living host” refers to the entire host or organism and not just a part excised (e.g., a liver or other organ) from the living host.

The term “hydrazino” refers to the group —NHNH₂. The term “substituted hydrazino” refers to the group —NRNR₂, wherein each R is independently hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, or —SO₂-substituted heterocyclic, or wherein two R groups may be joined, together with the nitrogen bound thereto, to form a heterocyclic or substituted heterocyclic group, provided that all three R groups are not hydrogen.

The term “isolated compound” means a compound that has been substantially separated from other compounds with which it occurs, e.g., in a synthetic preparation or, if a naturally occurring compound, in nature. Isolated compounds are usually at least about 80%, at least 90% pure, at least 98% pure, or at least about 99% pure, by weight. Purity percentages herein can also refer to purity in terms of other compounds present in a preparation, wherein, e.g., the 80% pure isolated compound contains 80 parts of the compound (and 20 parts of some other specified or unspecified compound(s) or material(s)). The present disclosure also includes diastereomers, racemic and resolved, enantiomerically pure forms, and pharmaceutically acceptable salts thereof.

“Optionally substituted” refers to “substituted or unsubstituted.”

The term “pharmaceutical composition” refers to a composition suitable for administration to a subject, such as a mammal, especially a human. In general a “pharmaceutical composition” is sterile, and free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The terms “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that is useful in preparing a pharmaceutical composition that is generally safe, non-toxic and neither biologically nor otherwise undesirable, and include an excipient, diluent, carrier, and adjuvant that is acceptable for veterinary use and/or human pharmaceutical use. “A pharmaceutically acceptable excipient, diluent, carrier and/or adjuvant” as used in the specification and claims includes one and/or more such excipients, diluents, carriers, and adjuvants.

The term “pharmaceutically acceptable salt” refers to a salt(s) that retains the biological effectiveness and optionally other properties of the free base(s) or acid(s) and that is obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like, or inorganic or organic bases. In the event that embodiments of the disclosed agents form salts, these salts are within the scope of the present disclosure. Reference to an agent of any of the formulas herein is understood to include reference to salts thereof, unless otherwise indicated. The term “salt(s)”, as employed herein, denotes acidic and/or basic salts formed with inorganic and/or organic acids and bases. In addition, when an agent contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of an agent may be formed, for example, by reacting the agent with an amount of acid or base, such as an equivalent amount, in a medium such as one in which the salt precipitates or in an aqueous medium followed by lyophilization. Embodiments of the agents that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like. Embodiments of the agents that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glutamines, N-methyl-D-glucamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Solvates of the agents of the disclosure are also contemplated herein.

The term “prodrug” refers to an inactive precursor of an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet, 15(2): 143-53; Balimane and Sinko (1999). Involvement of multiple transporters in the oral absorption of nucleoside analogues, Adv. Drug Delivery Rev., 39 (1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2 (1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The terms “prophylactically treat” or “prophylactically treating” refers completely or partially to preventing a disease or symptom thereof and/or may be therapeutic in terms of a partial or complete cure for a disease and/or adverse effect attributable to the disease.

The term “protected amino” refers to “substituted amino,” of formula —NHR^{y} or —N(R^{y})₂ wherein the R^{y} moiety or moieties can be removed by hydrogenolysis or acidic, basic or other chemical transformations well known to the skilled artisan, to provide an —NH₂ group or —NHR^{y} group.

The term “protected hydroxy” refers to —O—R^{z}, or —OCOR^{z}, or for phenolic hydroxy groups, also O—SO₂R^{z}, wherein R^{z} is a substituted or unsubstituted alkyl, alkenyl, alkynyl, cycloalkyl, heterocyclyl, aryl, or heteroaryl group, and the R^{z} moiety or moieties can be removed by hydrogenolysis or by acidic, basic or other chemical transformations well known to the skilled artisan, to provide an —OH group.

The term “protected carboxy” refers to carboxyl esters of formula —CO₂—R^{x}, wherein R^{x} is a substituted or unsubstituted alkyl, alkenyl, alkynyl, cycloalkyl, heterocyclyl, aryl, or heteroaryl group, and R^{x} may be converted to an H by hydrogenolysis or by acidic, basic or other chemical transformations well known to the skilled artisan, to provide a —CO₂H group.

The term “reduction” as used in connection with a symptom or symptoms (and grammatical equivalents of this phrase) refers to decreasing the severity or frequency of the symptom(s), or eliminating of the symptom(s).

The term “substituted” refers to a group as defined herein in which one or more bonds to a carbon(s) or hydrogen(s) are replaced by a bond to non-hydrogen and non-carbon atom “substituents” such as, but not limited to, a halogen atom; an oxygen atom in groups such as hydroxyl groups, alkoxy groups, aryloxy, and acyloxy groups; a sulfur atom in groups such as thiol groups, alkyl and aryl sulfide groups, sulfone groups, sulfonyl groups, and sulfoxide groups; a nitrogen atom in groups such as amino, alkylamines, dialkylamines, arylamines, alkylarylamines, diarylamines, alkoxyamino, hydroxyamino, acylamino, sulfonylamino, N-oxides, imides, and enamines; and other heteroatoms in various other groups. Any substituted group can be substituted with these functional groups, many of which are in addition to those specifically disclosed to define a particular “substituted group.” “Substituents” also include groups in which one or more bonds to a carbon(s) or hydrogen(s) atom is replaced by a higher-order bond (e.g., a double- or triple-bond) to a heteroatom such as oxygen in oxo, acyl, amido, alkoxycarbonyl, aminocarbonyl, carboxyl, and ester groups; nitrogen in groups such as imines, oximes, hydrazones, and nitriles. “Substituents” further include groups in which one or more bonds to a carbon(s) or hydrogen(s) atoms is replaced by a bond to a cycloalkyl, heterocyclyl, aryl, and heteroaryl groups. Another representative “substituent” is the trifluoromethyl group and other groups that contain the trifluoromethyl group. Typically, a particular group may have 0, 1, 2 or 3 substituents.

As used herein, for example, “substituted or unsubstituted alkyl, alkenyl, alkynyl, cycloalkyl, aryl, heteroaryl, or heterocyclyl group” refers to substituted or unsubstituted alkyl, substituted or unsubstituted alkenyl, substituted or unsubstituted alkynyl, substituted or unsubstituted cycloalkyl, substituted or unsubstituted aryl, substituted or unsubstituted heteroaryl, or substituted or unsubstituted heterocyclyl.

The term “substituted phenyl” refers to a phenyl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, substituted or unsubstituted, such that, for example, a biphenyl or naphthyl group results. Examples of the term “substituted phenyl” include a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “substituted amino” refers to monosubstituted amino (or (monosubstituted)amino or grammatical variants thereof), —NHR^{y}, or disubstituted amino (or (disubstituted)amino or grammatical variants thereof), —N(R^{y})₂, wherein R^{y} is substituted or unsubstituted alkyl, alkenyl, alkynyl, cycloalkyl, heterocyclyl, aryl heteroaryl, acyl, sulfonyl, sulfonyloxy, or protected carboxy group, or two R^{y} groups together with the nitrogen atom to which they are bonded form a substituted or unsubstituted heterocycle or a heteroaryl group.

The term “(substituted phenyl)alkyl” refers to a substituted phenyl groups attached to an alkyl group. Examples include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

The term “sulfonyl” refers to —SO₂R^{x}, wherein R^{x} is a substituted or unsubstituted alkyl, alkenyl, alkynyl, cycloalkyl, heterocyclyl, aryl, or heteroaryl group.

The term “sulfonyloxy” refers to —SO₃R^{x}, wherein R^{x} is a substituted or unsubstituted alkyl, alkenyl, alkynyl, cycloalkyl, heterocyclyl, aryl, or heteroaryl group.

The term “thioacyl” refers to —S-acyl.

The term “therapeutically effective amount” as used herein refers to that amount of an embodiment of the agent (which may be referred to as a compound, an inhibitory agent, and/or a drug) being administered that will relieve to some extent one or more of the symptoms of the disease, i.e., infection, being treated, and/or that amount that will prevent, to some extent, one or more of the symptoms of the disease, i.e., infection, that the host being treated has or is at risk of developing. Thus, the “therapeutically effective amount” is an amount administered to a patient with a disease, e.g., HCV infection, that is sufficient to effect beneficial or desired results. A therapeutically effective amount can be administered in one or more administrations, applications, or dosages.

The terms “treatment”, “treating”, and “treat” are defined as acting upon a disease, disorder, or condition with an agent to reduce or ameliorate the harmful or any other undesired effects of the disease, disorder, or condition and/or its symptoms. “Treatment,” as used herein, covers any treatment of a disease in a host (e.g., a mammal, typically a human or non-human animal of veterinary interest), and includes: (a) reducing the risk of occurrence of the disease in a subject determined to be predisposed to the disease but not yet diagnosed as infected with the disease, (b) impeding the development of the disease, and/or (c) relieving the disease, i.e., causing regression of the disease and/or relieving one or more disease symptoms, e.g., viral infection. “Treatment” also encompasses delivery of an inhibiting agent to provide a pharmacologic effect, even in the absence of a disease or condition. For example, “treatment” encompasses delivery of a disease or pathogen inhibiting agent that provides for enhanced or desirable effects in the subject (e.g., prevention of infection, reduction of pathogen load, reduction of disease symptoms, and the like). Thus, “treating” or “treatment of” a condition or patient refers to taking steps to obtain beneficial or desired results, including clinical results such as the reduction of symptoms. For purposes of this invention, beneficial or desired clinical results include, but are not limited to, alleviation or amelioration of one or more symptoms of HCV infection; prevention of HCV infection; diminishment of extent of HCV infection; delay or slowing of disease progression; amelioration, palliation, or stabilization of HCV infection; or other beneficial results.

The term “unit dosage form” as used herein, refers to physically discrete units suitable as unitary dosages for human and/or animal subjects, each unit containing a predetermined quantity of a compound (e.g., an anti-viral compound, as described herein) calculated in an amount sufficient to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the host.

The term “urea” refers to —NRCONR₂ wherein each R independently is hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, or —SO₂-substituted heterocyclic, or wherein 2 R groups may be joined, together with the nitrogen bound thereto, to form a heterocyclic or substituted heterocyclic group.

The term “urethane” refers to —O—CONR₂, wherein each R independently is hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, or substituted heterocyclic.

### II. Compounds of the Invention

In one aspect, the present invention provides compounds of Formula I:

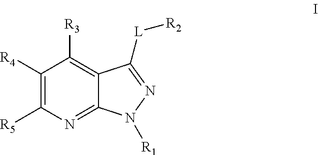

or a pharmaceutically acceptable salt thereof, wherein

R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;

L is a bond, —CONH—, —NH—CO—, substituted or unsubstituted C₁-C₅ alkylene, substituted or unsubstituted C₂-C₅ heteroalkylene, a substituted or unsubstituted 5 membered heteroaryl group, or a combination thereof;

R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group;

R₃, R₄, and R₅ are independently hydrogen, halo, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and

R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring.

To the extent that the compounds of the present invention, and salts thereof, may exist in their tautomeric forms, all such tautomeric forms are part of the present disclosure. All stereoisomers of the agents, such as those that may exist due to asymmetric carbons on the various substituents, including enantiomeric forms (which may exist even in the absence of asymmetric carbons) and diastereomeric forms, are within the scope of this disclosure. Individual stereoisomers of the compounds of the present invention may, for example, be substantially free of other isomers, or may be admixed, for example, as racemates or with all other, or certain other stereoisomers.

In one embodiment, R₃ is hydrogen, and R₄ and R₅ are both independently a non hydrogen substituent. In another embodiment, R₃ and R₅ are hydrogen, and R₄ is a non hydrogen substituent.

In another embodiment, the present invention provides a compound wherein R₃ and R₄ are hydrogen, and R₅ is a non hydrogen substituent. Thus, in one embodiment, the compound is of Formula IA:

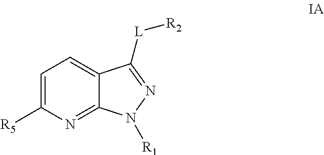

wherein L is —CO—NH—, —NH—CO—, —CO—NH—CH₂—, and —CH₂—Y—(CH₂)ₚ— wherein p is 0 or an integer from 1 to 4 and Y is a bond, —O— or —NH—, wherein the right hand side of each L moiety is attached to R₂. In one embodiment, L is —CO—NH—. In one embodiment, L is —NH—CO—. In one embodiment, L is —CO—NH—CH₂—. In one embodiment, L is —CH₂—Y—(CH₂)ₚ— wherein p is 1 or an integer from 1 to 4 and Y is —O— or —NH—. In one embodiment, Y is —O—. In one embodiment, Y is —NH—. In one embodiment, p is 0. In one embodiment, p is 1. In one embodiment, p is 2. In one embodiment, p is 3. In one embodiment, p is 4.

In another embodiment, the compound is of Formula IB:

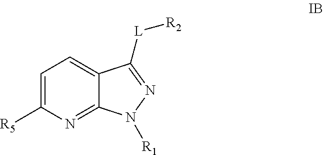

wherein:

R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO—(C₃-C₈ cycloheteroalkyl), —CO—(C₁-C₆ heteroalkyl), —SO₂—(C₁-C₆ cycloalkyl), or —SO₂—(C₃-C₈ cycloalkyl) group;

L is a bond, —CONH—, —NH—CO—, substituted or unsubstituted C₁-C₅ alkylene, substituted or unsubstituted C₂-C₅ heteroalkylene, or a combination thereof;

R₂ is a substituted or unsubstituted 5-7 membered heterocyclyl, C₅-C₇ cycloalkyl, 5-6 membered heteroaryl, or a 6 membered aryl group;

R₅ is R₅₁R₅₂N—, R₅₃(MeSO₂)N—, R₅₄O—, or substituted or unsubstituted C₁-C₆ alkyl;

R₅₁ is hydrogen or C₁-C₃ alkyl;

R₅₂ is C₁-C₃ alkyl, substituted or unsubstituted cycloalkyl, aryl, heterocyclyl, or heteroaryl group, wherein each cycloalkyl, aryl, heterocyclyl, or heteroaryl group contains 6-8 ring atoms, or R₅₁ and R₅₂ together with the nitrogen atom to which they are bonded form a 6, 7, 8, or 9-membered heterocyclyl ring containing up to 3 heteroatoms substituted by a substituted or unsubstituted benzyl, acyl, or sulfonyl group;

R₅₃ is substituted and unsubstituted C₁-C₆ alkyl; and

R₅₄ is hydrogen, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups.

In other embodiments, the present invention provides compounds of Formulas IC and ID:

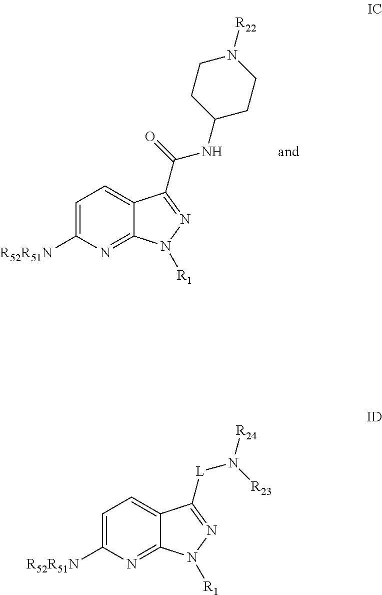

wherein R₁, R₂₂, R₂₃, R₂₄, R₅₁, and R₅₂ are defined as in any aspect or embodiment above (or below).

In another embodiment, R₁ is hydrogen, C₁-C₅ alkyl, or —(CH₂)ₖ—R₁₁; k is 1 or 2; and R₁₁ is C₃-C₈ cycloalkyl or a substituted or unsubstituted aryl or heteroaryl group. In another embodiment, R₁ is C₁-C₅ alkyl. In another embodiment, R₁ is hydrogen, methyl, ethyl, propyl, isopropyl, isobutyl, cyclopropylmethyl, or 4-chlorobenzyl. In another embodiment, R₁ is methyl. In another embodiment, R₁ is 4-chlorobenzyl. In another embodiment, R₁ is hydrogen, methyl, or 4-chlorobenzyl. In another embodiment, R₁ is hydrogen or methyl. In another embodiment, R₁ is hydrogen.

In another embodiment, L is —CONH— and the carbon atom of the —CO—NH— is bonded to the azaindazole ring.

In another embodiment, L is a substituted or unsubstituted C₁-C₅ alkylene or C₂-C₅ heteroalkylene group. In another embodiment, L is —(CH₂)ₙ—, —O—(CH₂)ₙ—, or —CH₂—O—(CH₂)ₙ— wherein, the left hand side of the L is bonded to the azaindazole moiety; and n is 1, 2, 3, or 4. In another embodiment, L is —(CH₂)ₙ—. In another embodiment, L is —O—(CH₂)ₙ—. In another embodiment, L is —CH₂—O—(CH₂)ₙ—. In another embodiment, n is 3 or 4. In another embodiment, n is 3 wherein L is —(CH₂)ₙ—. In another embodiment, R₁ is 4-chlorobenzyl, wherein L is —CH₂—O—(CH₂)ₙ— and n is 2 or 3.

In another embodiment, R₂ is substituted or unsubstituted piperidinyl, pyrrolidinyl, piperazinyl, or azepanyl group. In another embodiment, R₂ is a substituted or unsubstituted piperidin-3-yl or piperidin-4-yl group. In another embodiment, the substituted piperidin-4-yl group is:

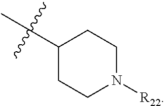

wherein R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂)ₙ—, or —CH₂—O—(CH₂)ₙ— wherein, the left hand side of the L is bonded to the azaindazole moiety and n is 1, 2, 3, or 4.

In other embodiments, R₂ may be a 4-piperidinyl group that is:

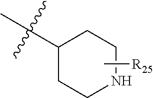

wherein R₂₅ is H or a substituent that is substituted or unsubstituted C₁-C₃ alkyl substituting a carbon or the nitrogen atom. In another embodiment, R₂ is piperidinyl of formula:

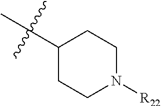

wherein R₂₂ is C₃-C₁₅ alkenyl, C₁-C₄ alkyl optionally substituted with a piperidine or a cyclohexyl moiety, substituted or unsubstituted benzyl, or C₅-C₈ cycloalkyl.
In another embodiment, R₂ is:

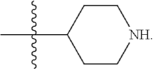

In another embodiment, R₂ is:

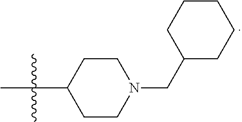

In another embodiment, R₂ is:

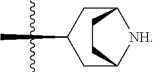

In another embodiment, R₂ is:

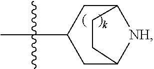

wherein k is 1 or 2.
In another embodiment, R₂ is:

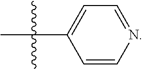

In another embodiment, R₂ is:

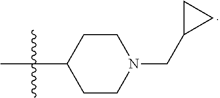

In another embodiment, R₂ is:

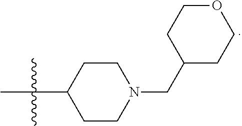

In another embodiment, R₂ is:

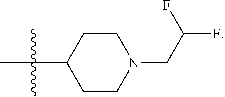

In another embodiment, R₂ is:

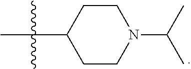

In another embodiment, R₂ is:

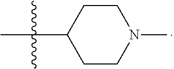

In another embodiment, R₂ is:

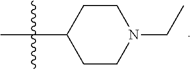

In another embodiment, R₂ is:

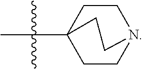

In another embodiment, R₂ is substituted or unsubstituted thienyl.

In another embodiment, NR₂₃R₂₄ is:

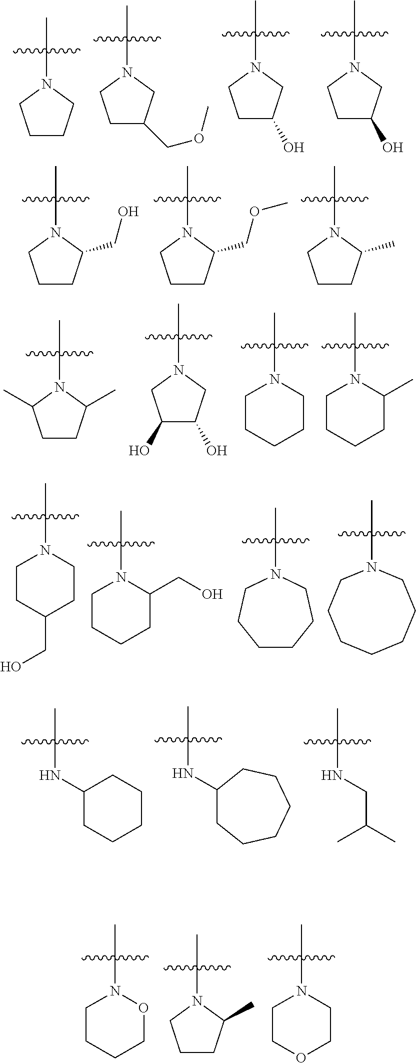

In another embodiment, NR₂₃R₂₄ is:

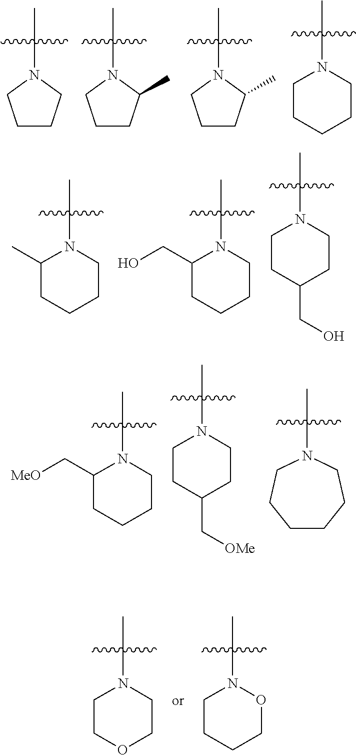

In another embodiment, NR₂₃R₂₄ is:

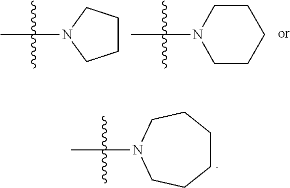

In another embodiment, R₅ is —NR₅₁R₅₂, wherein R₅₁ is H, methyl, or ethyl and R₅₂ is ethyl, isobutyl, cyclohexyl, cycloheptyl, cyclooctyl, or cyclohexylmethyl, or —NR₅₁R₅₂ is:

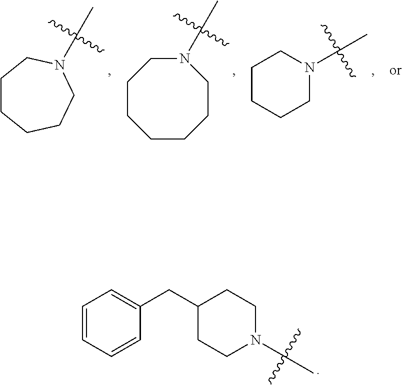

In another embodiment, —NR₅₁R₅₂ is:

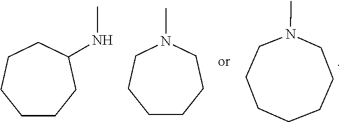

In another embodiment, R₅ is —NR₅₁R₅₂, which is:

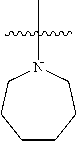

In another embodiment, R₅₃ is HOCH₂CH₂(MeSO₂)N—.

In another embodiment, R₅ is —OR₅₄, which is:

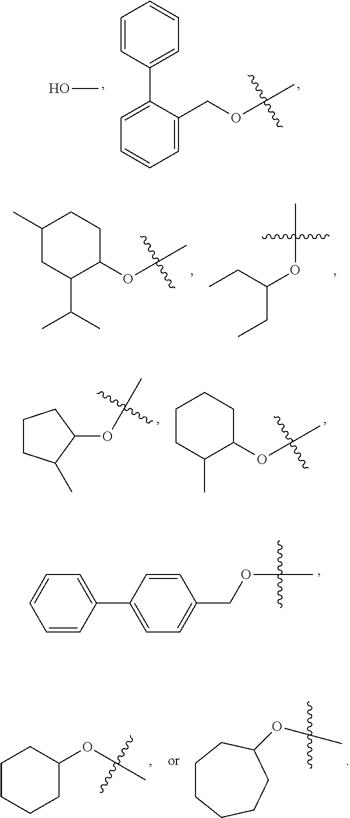

In another embodiment, among the compounds of the present invention, particularly chosen, e.g., and without limitation, for their impressive in vitro and in vivo properties, are compounds of Formula IC, wherein R₅₁R₅₂N— is a azepanyl or a similar medium ring (containing 7-8 ring atoms) heterocycle, R₁ is methyl or closely related lower alkyl (such as a C₂-C₃ alkyl group), and R₂₂ is ethyl, isopropyl, or a ethyl substituted with a 5 or 6 membered heterocycle ring containing a basic nitrogen atom. Exemplary such compounds include, without limitation EBP1047, EBP1595, EBP1597, and EBP1604.

In another embodiment, the present invention provides an isolated compound, which is EBP841, EBP1310, EBP1047, EBP1489, EBP1597, EBP1452, EBP1172, or EBP1456, whose structures are shown below, or a pharmaceutically acceptable salt or prodrug of each thereof.

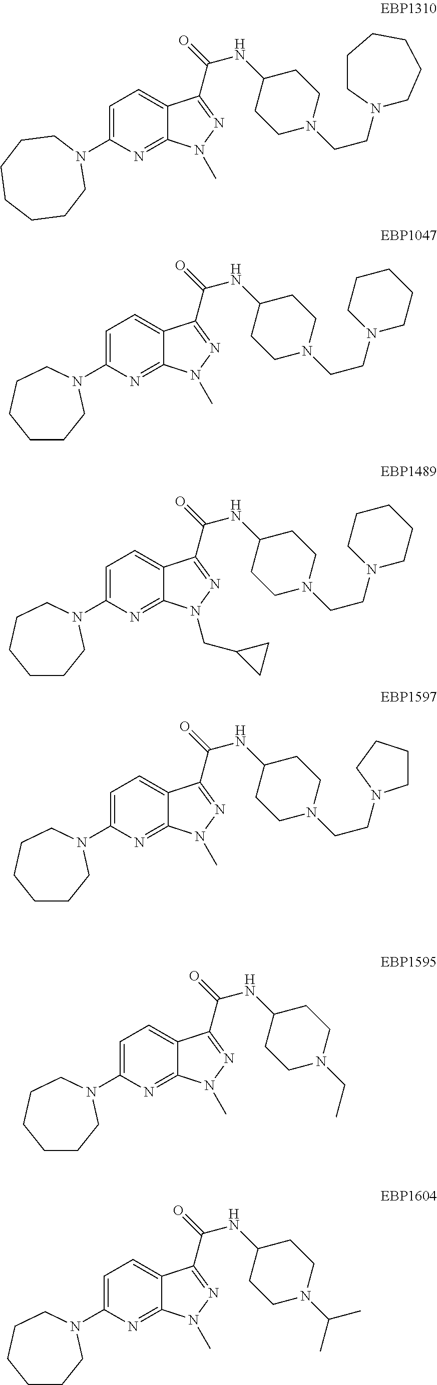

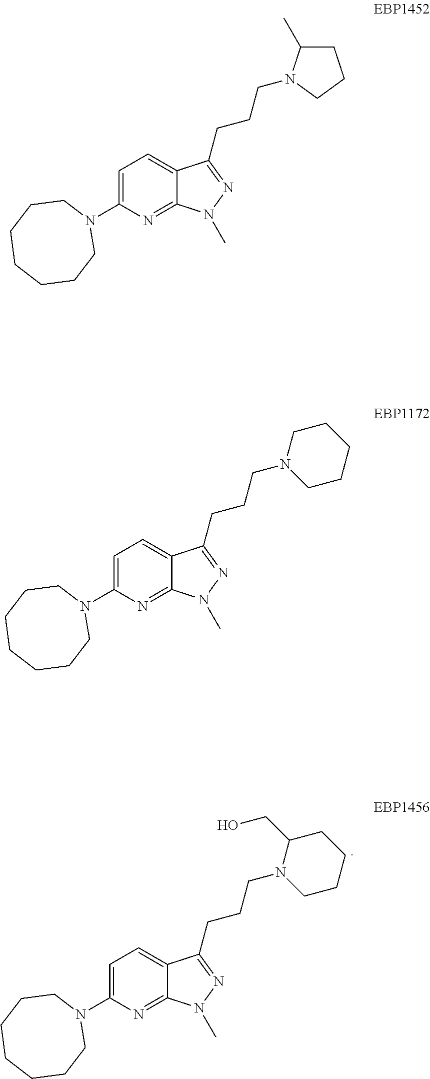

In one embodiment, such compounds of the present invention are useful in inhibiting hepatitis C virus (HCV), including, without limitation, genotypes 4, 2a and/or 1b of HCV.

In another embodiment, for each compound within the scope of Formula I, IA and IB, R₅ is R₅₁R₅₂N— or R₅₄O—; R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 7, 8, or 9-membered heterocyclyl ring containing in total 1 nitrogen atom and R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups.

In one embodiment, the present invention provides compounds of Formula II, shown below:

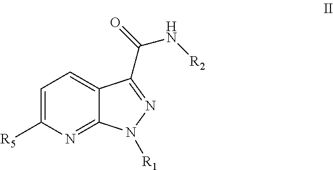

wherein, R₁ is hydrogen, branched or linear C₁-C₅ alkyl, C₂-C₁₅ alkenyl, unsubstituted or substituted cycloalkyl, —CO-(cycloalkyl), —SO₂— (cycloalkyl) group, or —(CH₂)ₙ—R₁₁, or R₅ and R₁ together form a 12-18 membered heterocycle; n is 1 or 2; R₂ is substituted or unsubstituted piperidinyl, 4-pyridyl, pyrrolidinyl, piperazinyl, benzyl, substituted phenyl, or pirazolyl group; R₅ is R₅₁R₅₂N— or R₅₄O—; R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁₁ is C₅-C₈ cycloalkyl or substituted or unsubstituted aryl, or substituted or unsubstituted heteroaryl. In various embodiments, the compound of Formula II can have R₁, R₂, and R₃ groups as defined below.

In another embodiment of Formula II, R₁ is C₁-C₃ alkyl. In one embodiment, R₁ is methyl. In one embodiment, R₁₁ is cyclohexyl. In one embodiment, R₁₁ is halo substituted phenyl. In one embodiment, R₁₁ is 2-chlorophenyl or 3-chlorophenyl.

In another embodiment of Formula II, R₁ is substituted or unsubstituted 4-piperidinyl or 3-piperidinyl group. In various embodiments, R₂ is a 4-piperidinyl group that is:

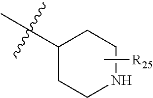

wherein R₂₅ is H or a substituent that is substituted or unsubstituted C₁-C₃ alkyl substituting a carbon or the nitrogen atom. In another embodiment, R₂ is piperidinyl of formula:

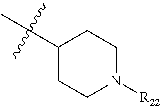

wherein R₂₂ is C₃-C₁₅ alkenyl, C₁-C₄ alkyl optionally substituted with a piperidine or a cyclohexyl moiety, substituted or unsubstituted benzyl, or C₅-C₈ cycloalkyl.
In another embodiment R₂ is

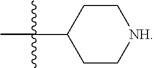

In another embodiment, R₂ is:

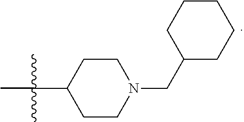

In another embodiment, R₂ is:

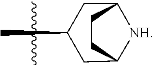

In another embodiment, R₂ is:

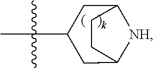

wherein k is 1 or 2.
In another embodiment, R₂ is:

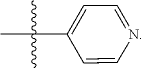

In another embodiment, R₂ is:

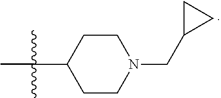

In another embodiment, R₂ is:

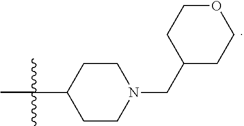

In another embodiment, R₂ is:

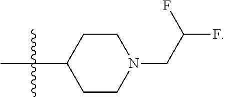

In another embodiment, R₂ is:

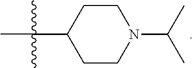

In another embodiment, R₂ is:

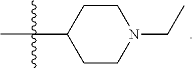

In another embodiment, R₂ is:

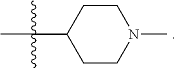

In another embodiment, R₂ is:

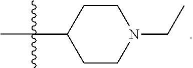

In another embodiment, R₂ is:

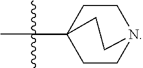

In another embodiment, R₂ is substituted or unsubstituted thienyl.

In another embodiment of Formula II, R₅ is R₅₁R₅₂N— or R₅₄O—; R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 7, 8, or 9-membered heterocyclyl ring containing in total 1 nitrogen atom and R₂₃ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups.

In one embodiment, the present invention provides compounds of Formula IIA, shown below:

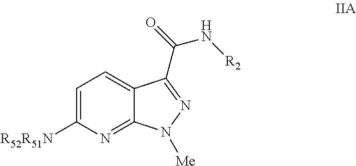

wherein R₂ and NR₅₁R₅₂ are defined as in Formula II above. In another embodiment, R₂ is substituted or unsubstituted 3-piperidinyl or 4-piperidinyl. In another embodiment, R₂ is 4-pyridyl. In another embodiment, —NR₅₁R₅₂ is

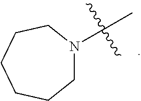

In another embodiment, the present invention provides compounds of Formula IIB, shown below:

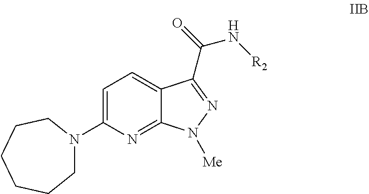

wherein R₂ is defined as in Formula II above.

In another embodiment, the present invention provides compounds of Formula IIC, shown below:

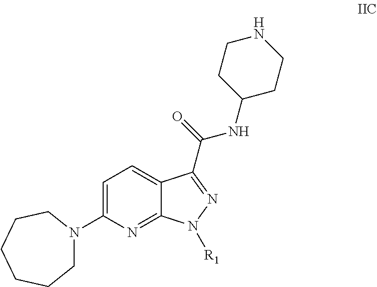

wherein R₁ is defined as in Formula II above.

In another embodiment, the present invention provides compounds of Formula IID, shown below:

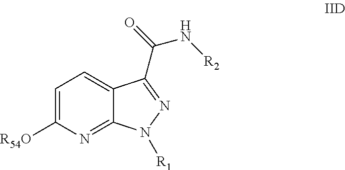

wherein R₂ is substituted or unsubstituted 4-piperidinyl or 3-piperidinyl group; R₁ is defined as in Formula II above; and R₅₄ is substituted or unsubstituted benzyl, 3-pentyl, cyclopentyl or cyclohexyl substituted with up to 2 branched or linear C₁-C₃ alkyl group, or cycloheptyl. In another embodiment of Formula IID, R₅₄ is phenyl, cyclopentyl, cyclohexyl, or cycloheptyl.

In another embodiment, the present invention provides compounds of Formula IIE, shown below:

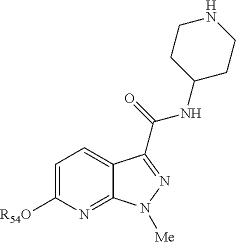

wherein R₅₄ is defined as in Formula II above.

In another embodiment, the present invention provides compounds of Formula IIF, shown below:

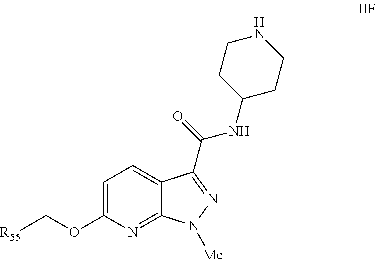

wherein R₅₅ is substituted or unsubstituted phenyl. In one embodiment, R₅₅ is phenyl. In another embodiment, R₅₅ is ortho or 2-substituted phenyl, substituted with halo or substituted or unsubstituted aryl or heteroaryl.

In another embodiment, the present invention provides compounds of Formula IIG, shown below:

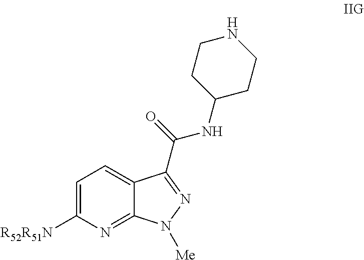

wherein NR₅₁R₅₂ is defined as in Formula IB or II above or is 1-azepanyl or formula

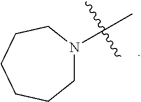

In another embodiment, the present invention provides compounds having the structure of Formula IIH, shown below:

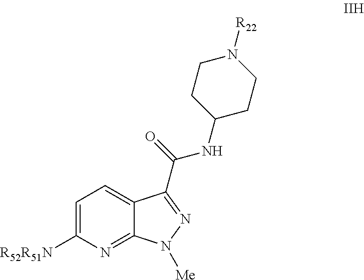

wherein NR₅₁R₅₂ is defined as in Formula IB or II above or is 1-azepanyl, and R₂₂ is hydrogen or substituted or unsubstituted C₁-C₆ alkyl.

In another embodiment, the present invention provides compounds of Formula II-I, shown below:

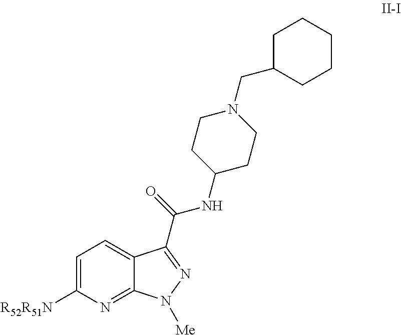

wherein NR₂₁R₂₂ is defined as in Formula IB or II above, or is 1-azepanyl.

In another embodiment, the present invention provides compounds of Formula III, shown below:

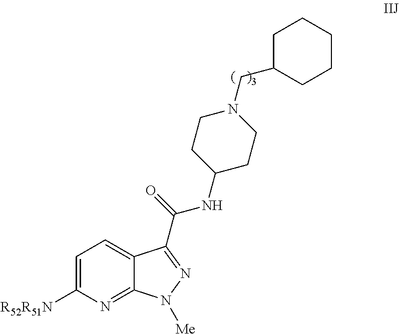

wherein NR₅₁R₅₂ is defined as in Formula IB or II above, or is 1-azepanyl.

In another embodiment, the present invention provides compounds of Formula IIK, shown below:

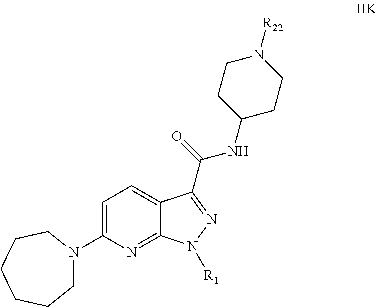

wherein R₂₂ is hydrogen or substituted or unsubstituted C₁-C₆ alkyl. In another embodiment, the R₂₂ substituent is —COR₂₆ wherein R₂₆ is substituted or unsubstituted alkyl, aryl, cycloalkyl, heteroaryl, or heterocyclyl.

In another embodiment, the present invention provides compounds of Formulas III and IIIA, shown below:

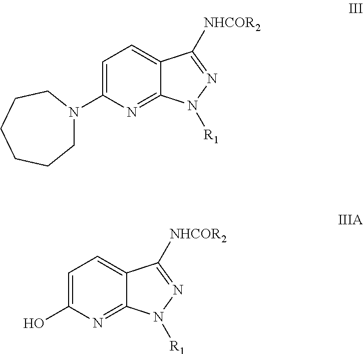

wherein R₂ is 3-pyridyl or 4-pyridyl, 3-pyridyl or 4-pyridyl independently substituted with chloro, phenyl, monosubstituted phenyl, substituted or unsubstituted thienyl, or —(CH₂)_{q}—R₂₇, wherein q is 0 or 1; R₂₇ is unsubstituted cyclohexyl, cyclohexyl substituted with an amino group, or piperidinyl; R₁ is methyl, hydrogen, or 4-chlorobenzyl; provided however that when R₁ is 4-chlorobenzyl or hydrogen, then q is 0 and R₅ is 3-piperidinyl or 4-piperidinyl.

In another embodiment, the present invention provides compounds of Formula IV, shown below:

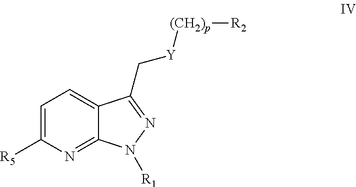

wherein R₂ is substituted or unsubstituted 5-8 membered heterocyclyl group containing 1 nitrogen atom; Y is oxygen, NH or a bond; p is 0 or an integer from 1-4; R₅ is —NR₅₁R₅₂ or —OR₅₄ wherein R₅₁-R₅₂ is defined as in any one of the Formula above; and R₁ is methyl or 4-chlorobenzyl. In another embodiment, R₂ is piperidinyl; Y is oxygen or NH; p is 0 or an integer from 1-4, R₅ is

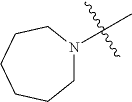

or —O—R₅₄; R₅₄ is 3-pentyl; and R₃ is methyl or 4-chloronebenzyl. In another embodiment, R₂ is 1-, 3-, or 4-piperidinyl.

In another embodiment, the present invention provides compounds of Formula IV having the following Formulas, wherein n and m are 1-4:

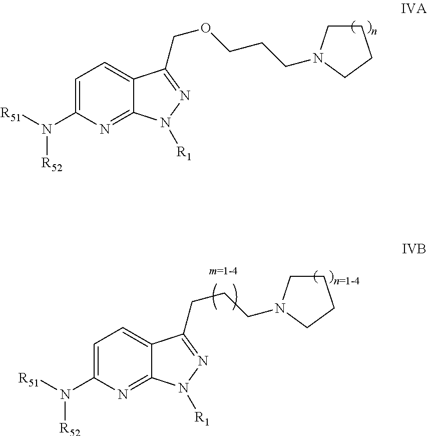

In another embodiment, the present invention provides compounds of Formulas V and VA, shown below:

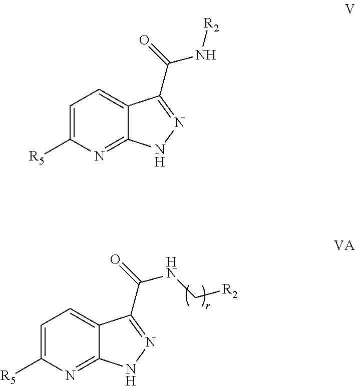

wherein R₂ is piperidinyl or piperidinyl (3- or 4-) substituted with a C₁-C₃ alkyl group or piperidinylmethyl; R₅ is Me(Me₂CHCH₂)N—,

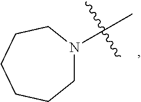

cyclohexyl-O—, or 3-pentyl-O—, and r is 0 or 1. In another embodiment, R₂ is 3- or 4-piperidinyl. In another embodiment, R₂ is 3- or 4-piperidinyl substituted with up to 3 C₁-C₃ alkyl group.

In another embodiment, the present invention provides compounds of Formula VB, shown below:

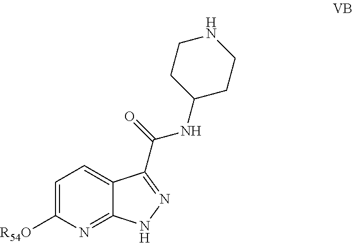

wherein R₅₄ is defined as in Formula, IB, II, or IID above.

Within these and other embodiments, in one embodiment, R₁ is hydrogen, in another embodiment, R₁ is methyl, in another embodiment, R₁ is 4-chlorobenzyl, in another embodiment, R₁ is 3-pentyl, and in yet another embodiment, R₁ is —CO-cyclohexyl. Within these and other embodiments, in one embodiment L is —CO—NH—, —NH—CO—, —CO—NH—CH₂—, and —CH₂—Y—(CH₂)ₚ— wherein p is 1 or an integer from 1 to 4 and Y is —O— or —NH—, wherein the right hand side of each L moiety is attached to R₂. In one embodiment, L is —CO—NH—. In one embodiment, L is —NH—CO—. In one embodiment, L is —CO—NH—CH₂—. In one embodiment, L is —CH₂—Y—(CH₂)ₚ— wherein p is 1 or an integer from 1 to 4 and Y is —O— or —NH—. In one embodiment, Y is —O—. In one embodiment, Y is —NH—. In one embodiment, p is 0. In one embodiment, p is 1. In one embodiment, p is 2. In one embodiment, p is 3. In one embodiment, p is 4. In various embodiments where R₁ is defined as in this paragraph, -L- is —CO—NH— where the —NH— moiety of L is attached to the R₁ group which is a substituted or unsubstituted 4-piperidinyl or 3-piperidinyl group. In various embodiments, the 4-piperidinyl group is

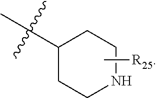

wherein R₂₅ is H or substituent that is substituted or unsubstituted C₁-C₃ alkyl substituting a carbon or the nitrogen atom. In other embodiments, the 3-piperidinyl group is

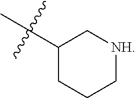

In another embodiment, the present invention provides compounds of Formulas VI, VIA, and VIB:

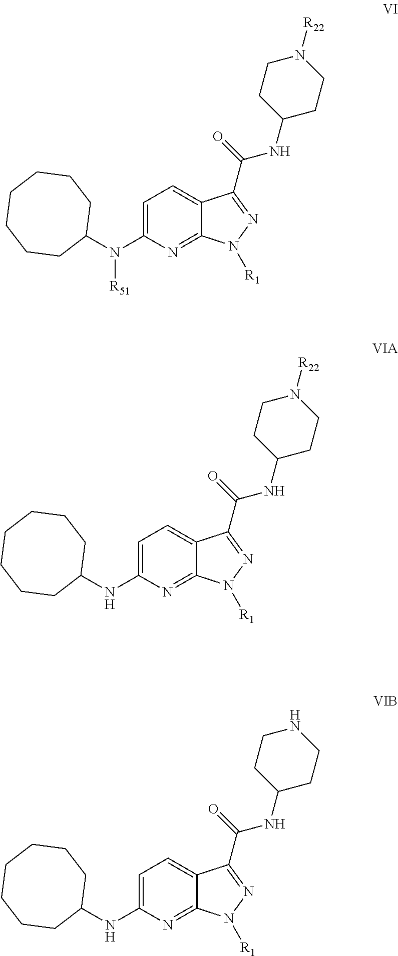

wherein R₁ and R₅₁ are defined as in Formula IB or II above and R₂₂ is hydrogen or substituted or unsubstituted C₁-C₆ alkyl.

In another embodiment, the present invention provides compounds of Formulas VII and VIIA:

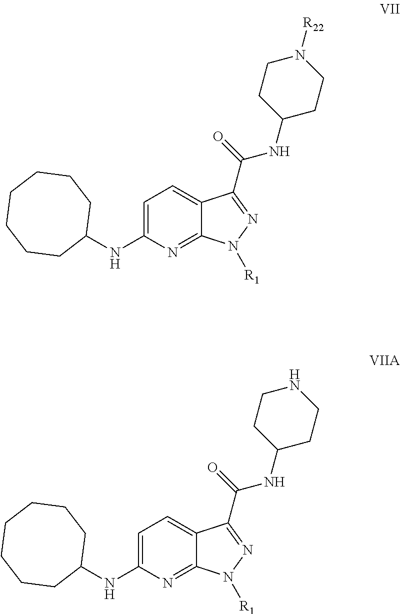

wherein R₁ is defined as in Formula I or II above and R₂₂ is hydrogen or substituted or unsubstituted C₁-C₆ alkyl.

In another embodiment, the present invention provides compounds of Formulas VIII and VIIIA:

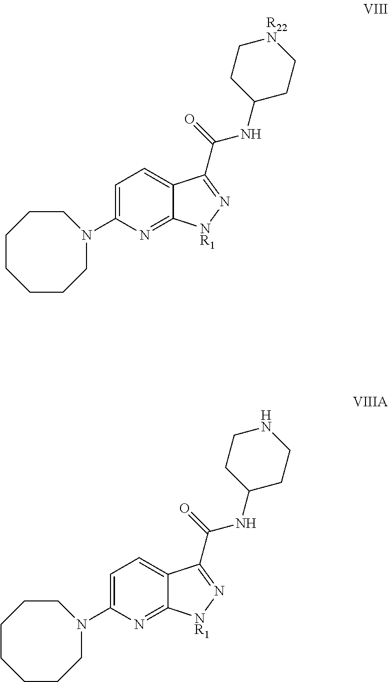

wherein R₁ is defined as in Formula I or II above and R₂₂ hydrogen or substituted or unsubstituted C₁-C₆ alkyl.

In another embodiment, the present invention provides compounds of Formula IX:

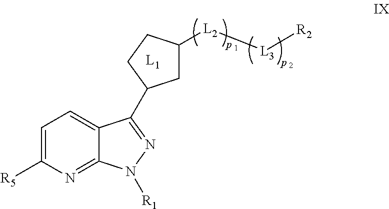

wherein L₁ is 5 membered heteroaryl containing up to 3 heteroatoms selected from the group consisting of O, N, and S; L₂ is —CO—NH— wherein the carbon atom is attached to L₁; L₃ is substituted or unsubstituted C₁-C₃ alkylene; p₁ is 0 or 1; p₂ is 0, 1, or 2; R₂ is 3- or 4-piperidinyl; R₅ is —NR₅₁R₅₂ or —OR₅₄ wherein R₅₁, R₅₂, and R₅₂ is defined as in any one of the formula above; and R₁ is methyl. In another embodiment, P₁ is 1 and P₂ is 0 or 1. In another embodiment, P₁ is 0 and P₂ is 0 or 1. In another embodiment, P₂ is 0. In another embodiment, P₂ is 1. In another embodiment, L₃ is —CH₂—. In other embodiments, the compounds of Formula IX have the following Formulas, wherein R₇, R₈, and R₉ are independently hydrogen or substituted or unsubstituted C₁-C₃ alkyl:

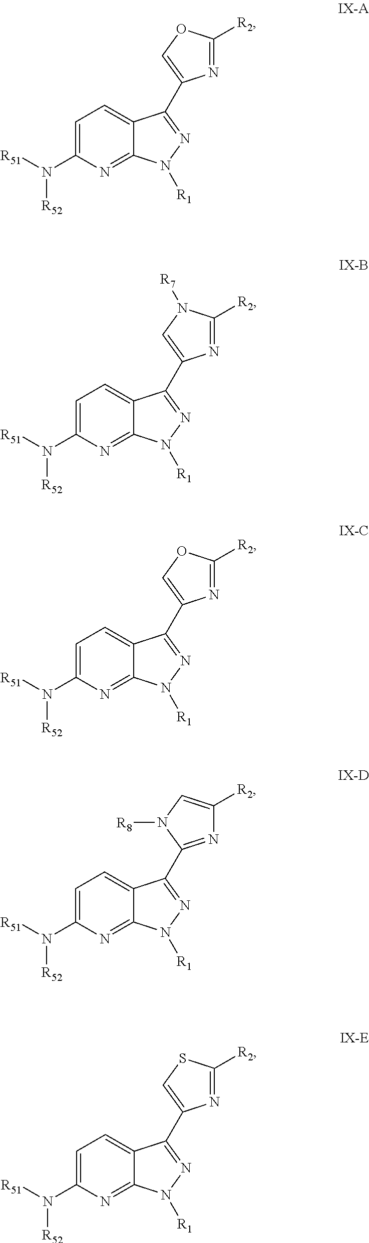

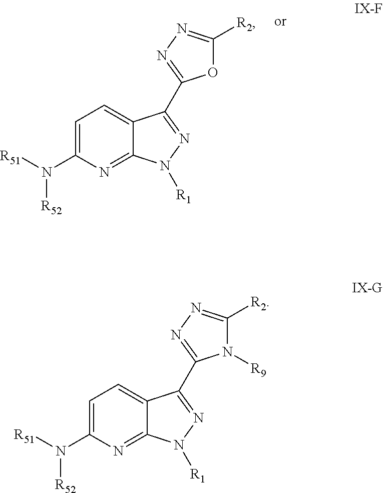

In another embodiment, the present invention provides compounds that are isolated compounds. In another embodiment, the isolated compounds are at least about 80%, at least about 90% pure, at least about 98% pure, or at least about 99% pure.

In another embodiment, the present invention provides compounds shown in the tables below and pharmaceutically acceptable salts or prodrugs thereof. The anti-HCV activity of these compounds, as measured by their EC₅₀ against HCV genotype 1b, and their hERG activities are also shown, if available. Preferred compounds of the invention include those compounds having an anti-HCV 1b activity (as shown in the table) of less than about 4 microMolar (“μM”), including but not limited to those having an EC₅₀ less than about 1 μM, hERG activity of greater than about 10 μM, permeability Papp (A-B)>5 μM, and efflux/permeability ratio of Papp (B-A)/Papp (A-B)<3. A method for demonstrating the activity of compounds against HCV genotype 1b and 2a and hERG is described in the Examples section below. Methods for assaying compounds for their activity against HCV genotype 2a and hERG are also generally known in the art.

In one embodiment, the present invention provides compounds included in Table 1, which have, or are expected to have, EC₅₀ values less than or equal to 25 micromolar in the HCV 1b replicon assay.

TABLE 1

| | | 1b EC₅₀ | hERG | |
|---|---|---|---|---|
| EBP # | Structure | (μM) | (μM) | Other Data |
| 655 | 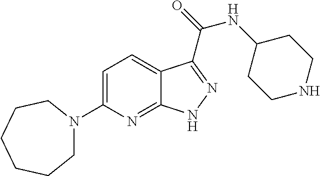 | 2.1-3.5 | 1.34 | |
| 658 | 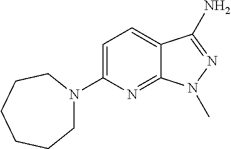 | 0.4-2.2 | >10 | |
| 659 | 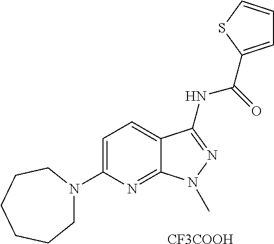 | 1.8-4.9 | >10 | |
| 680 | 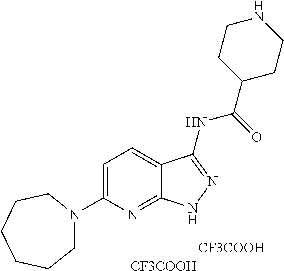 | 4.1 | >10 | |
| 681 | 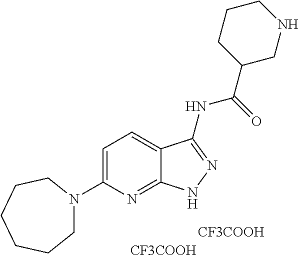 | 8.5 | | |
| 684 | 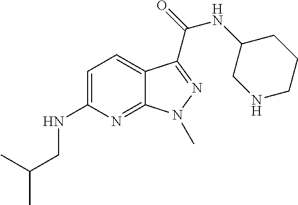 | 10 | | |
| 686 | 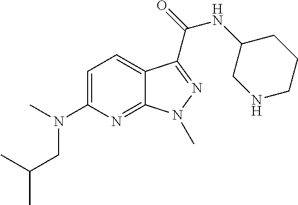 | 2.6 | | |
| 688 | 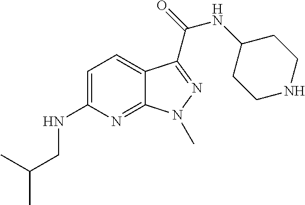 | 4.9 | | |
| 690 | 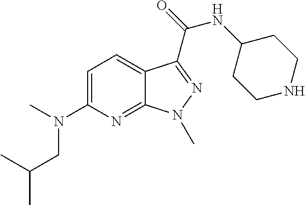 | 1.2 | | |
| 697 | 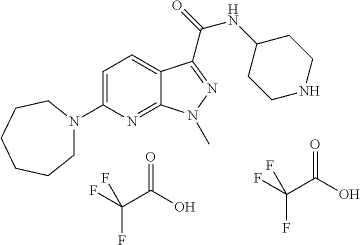 | 0.67 | >10 | Efflux/permeability = 48/5.5; human liver microsome (HLM, % parent remaining): 96, 109, 108; rat liver microsome (RLM): 69, 42, 13; mouse liver microsome (MLM): 85, 74, 44; CYP (% negative controls) 1A2 = 97; 2C9 = 92; 2C19 = 78; 2D6 = 80; 3A4 = 110 |
| 698 | 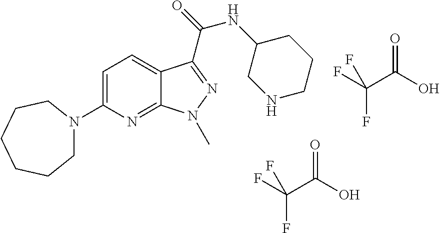 | 3.8 | | |
| 699 | 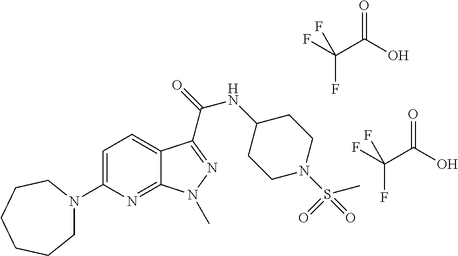 | 3.8 | | |
| 700 | 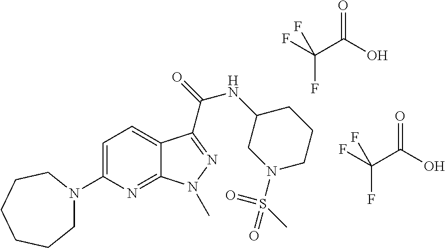 | 3.8 | | |
| 701 | 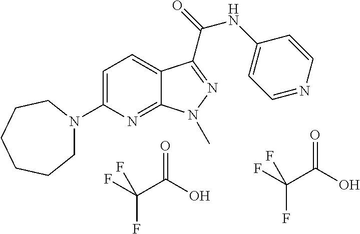 | 1.1 | >10 | |
| 702 | 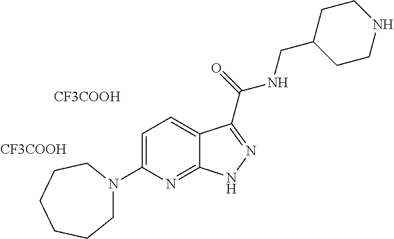 | 3.4 | >10 | |
| 703 | 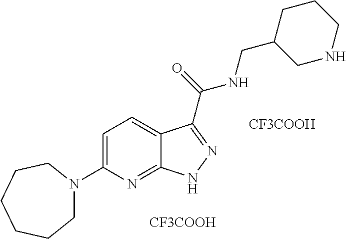 | 2.2 | 8.8 | |
| 704 | 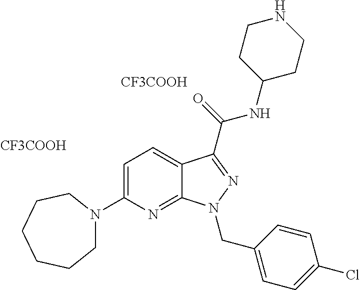 | 2.5 | | |
| 705 | 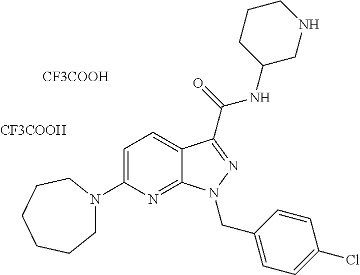 | 2.7 | | |
| 707 | 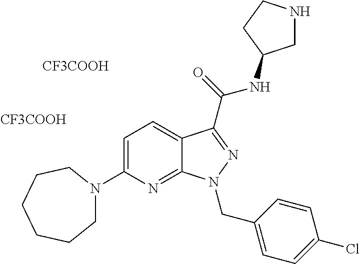 | 3.3 | | |
| 708 | 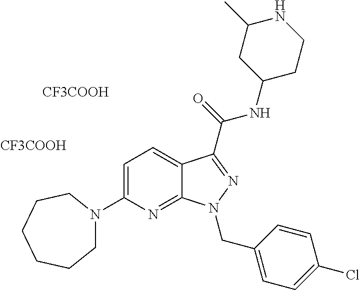 | 2.4 | | |
| 709 | 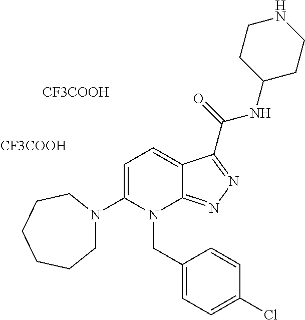 | 8.7 | | |
| 710 | 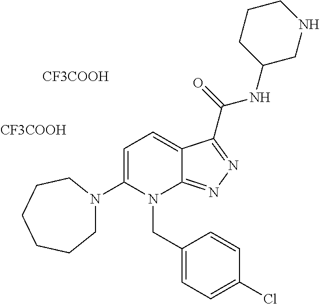 | 7.9 | | |
| 712 | 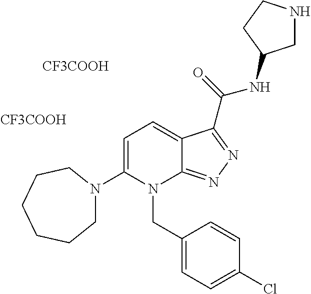 | 8.2 | | |
| 713 | 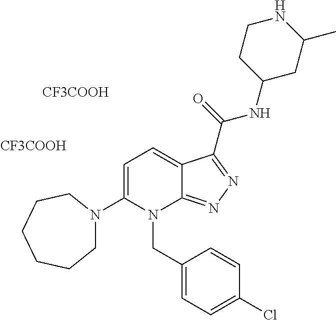 | 6.1 | | |
| 725 | 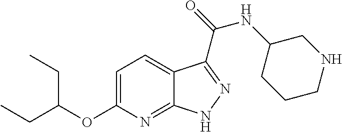 | 7.6 | | |
| 726 | 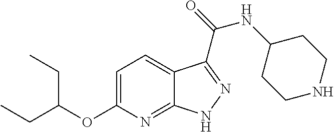 | 1.2 | >10 | Efflux/permeability = 41/3.5; human liver microsome (HLM, % parent remaining): 86, 81, 65; rat liver microsome (RLM): 83, 69, 53; mouse liver microsome (MLM): 99, 96, 86; CYP (% negative controls) 1A2 = 102; 2C9 = 84; 2C19 = 74; 2D6 = 72; 3A4 = 60 |
| 728 | 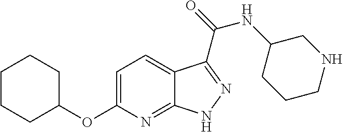 | 8 | | |
| 729 | 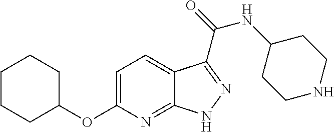 | 1.8 | >10 | |
| 734 | 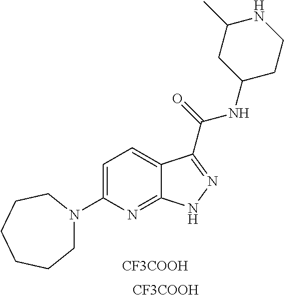 | 2.6 | | |
| 738 | 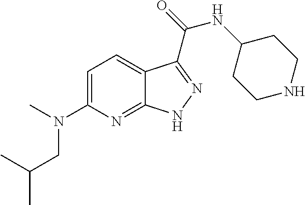 | 2.2 | >10 | |
| 744 | 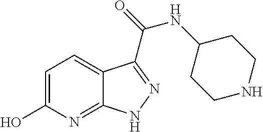 | 1.5 | >10 | |
| 745 | 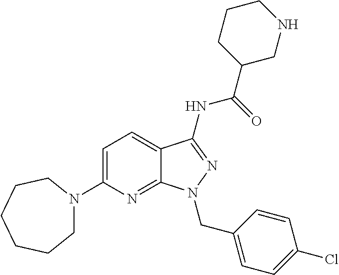 | 3 | >10 | |
| 747 | 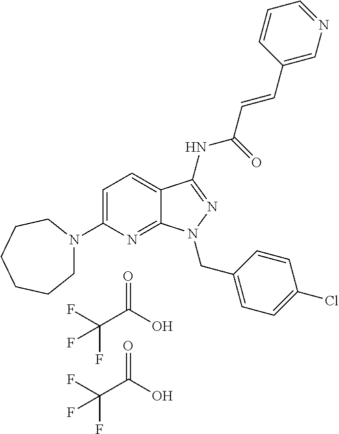 | 9 | | |
| 749 | 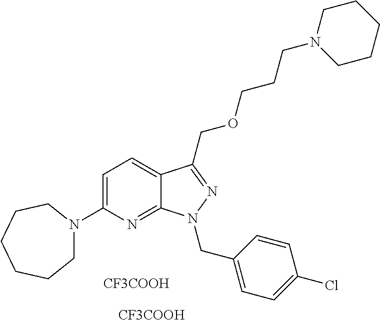 | 2 | 3.3 | Efflux/permeability = 0.52/0.44 |
| 751 | 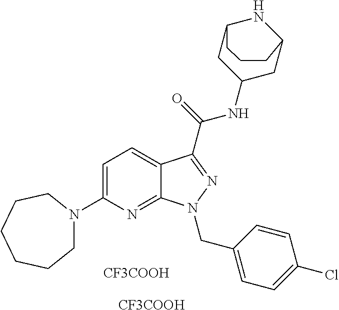 | 2 | | |
| 755 | 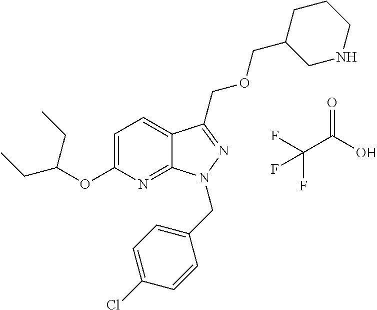 | 3.2 | >10 | Efflux/permeability = 0.2/0.4 |
| 756 | 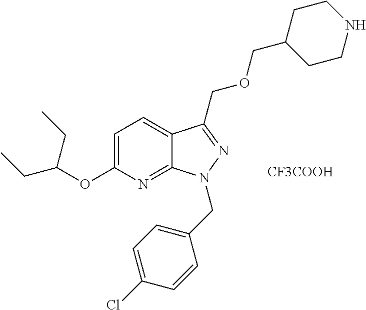 | 2.2 | 10 | |
| 759 | 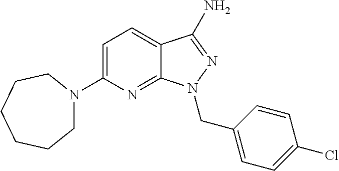 | 8.6 | | |
| 762 | 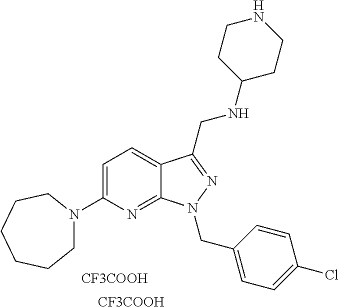 | 2.9 | | |
| 763 | 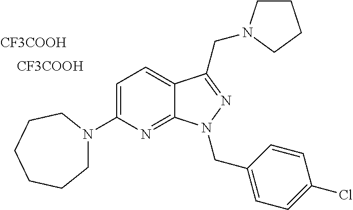 | 8.7 | | |
| 777 | 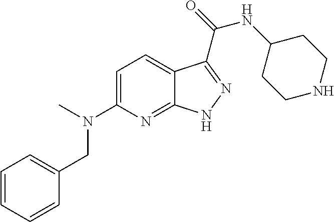 | 2 | | |
| 778 | 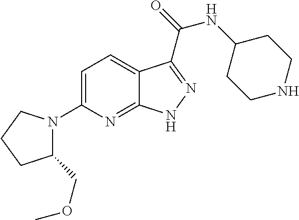 | 17 | | |
| 788 | 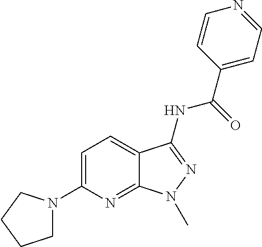 | 9.5 | | |
| 791 | 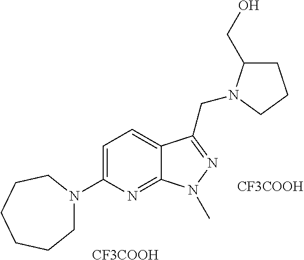 | 8.5 | | |
| 792 | 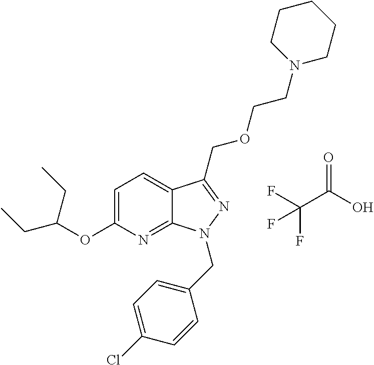 | 4.6 | | |
| 794 | 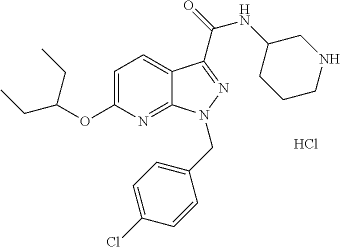 | 2.8 | | |
| 795 | 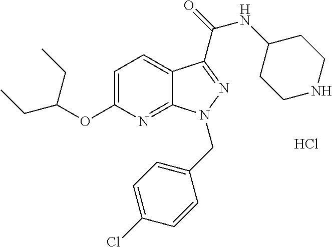 | 3.2 | | |
| 811 | 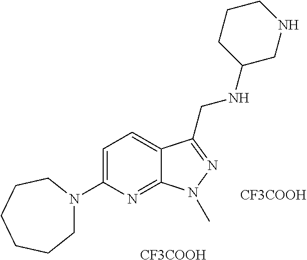 | 2.9 | | |
| 812 | 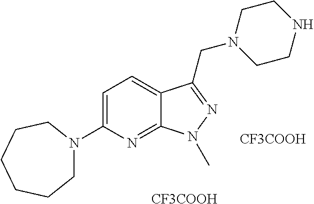 | 4.6 | | |
| 813 | 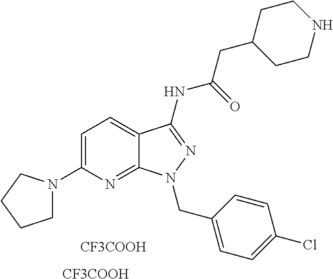 | 6.8 | | |
| 815 | 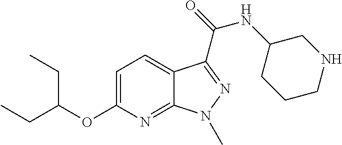 | 6.6 | | |
| 816 | 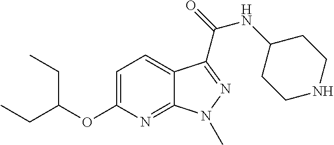 | 2.4 | | |
| 817 | 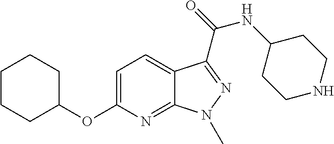 | 3.4 | | |
| 822 | 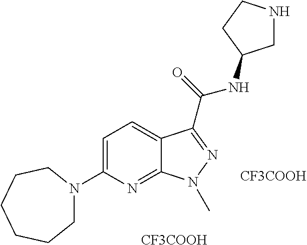 | 4.3 | | |
| 823 | 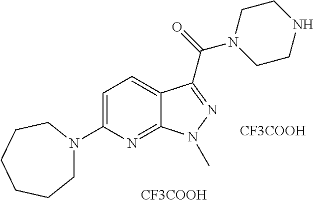 | 7.6 | | |
| 824 | 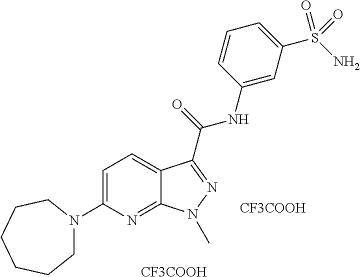 | 3.5 | | |
| 827 | 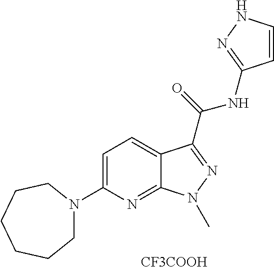 | 3.2 | | |
| 829 | 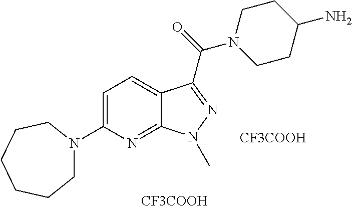 | 8.7 | | |
| 830 | 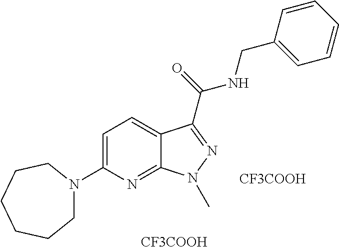 | 7.3 | | |
| 831 | 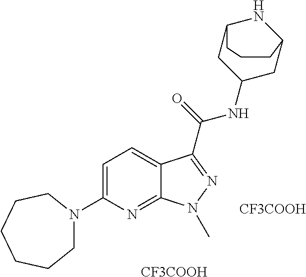 | 2.7 | | |
| 832 | 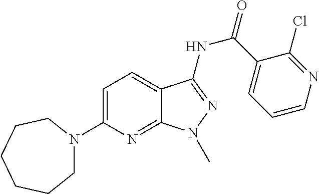 | 3.3 | | |
| 833 | 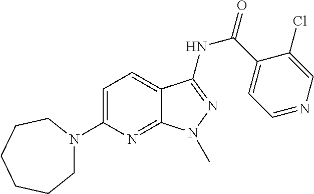 | 2.9 | | |
| 835 | 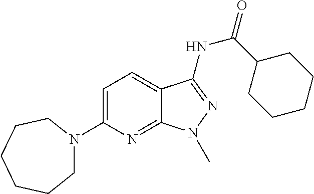 | 2.4 | | |
| 836 | 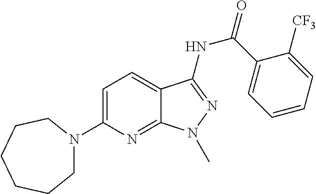 | 4.1 | | |
| 838 | 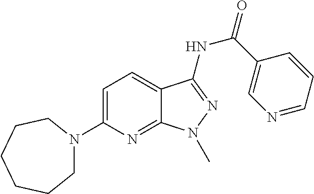 | 3.8 | | |
| 839 | 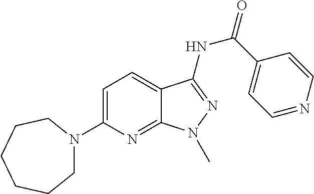 | 2.5 | | |
| 841 | 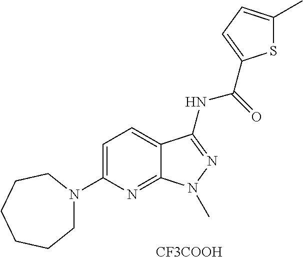 | 1.6 | | Efflux/permeability = 1.9/9.7 |
| 843 | 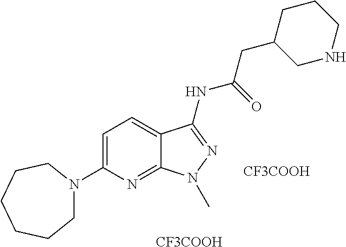 | 8.3 | | |
| 844 | 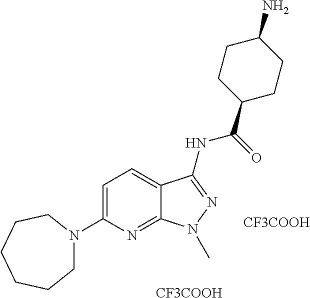 | 6.6 | | |
| 845 | 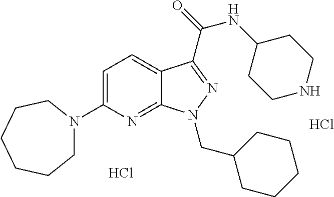 | 2 | | |
| 850 | 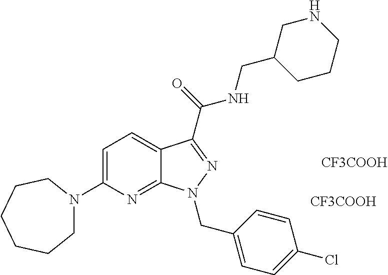 | 2.5 | | |
| 852 | 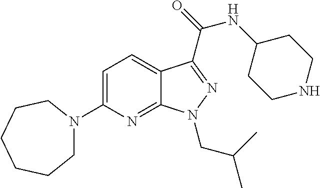 | 2.9 | | |
| 853 | 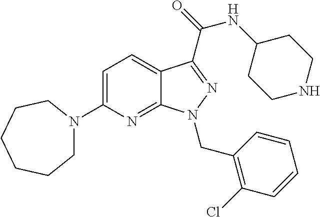 | 2.9 | | Efflux/permeability = 0.4/0.4 |
| 854 | 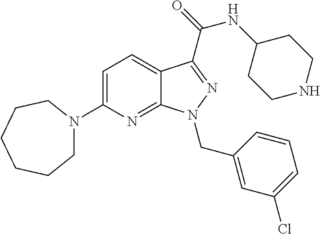 | 7.2 | | |
| 855 | 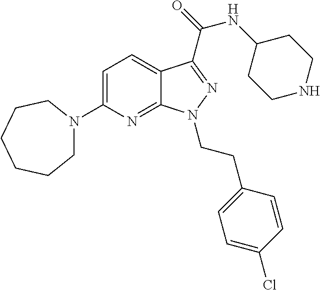 | 3.7 | | |
| 856 | 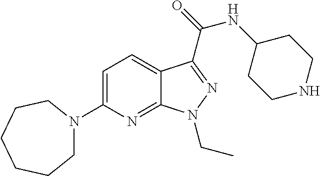 | 3.7 | | |
| 857 | 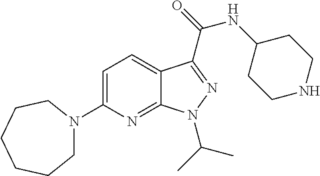 | 3.1 | | |
| 858 | 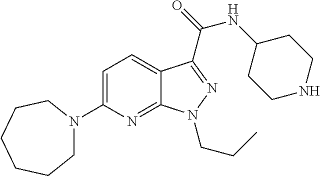 | 3.3 | | |
| 859 | 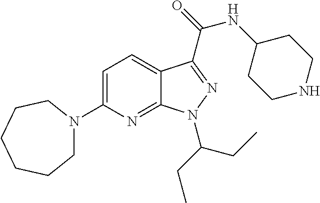 | 2.8 | | |
| 860 | 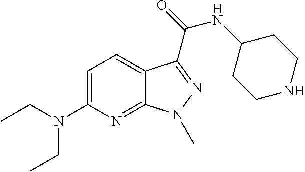 | 2.9 | | |
| 861 | 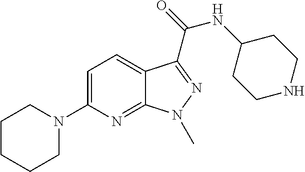 | 7.3 | | |
| 862 | 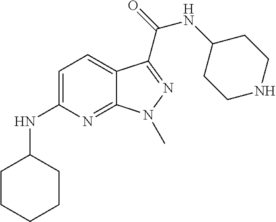 | 2.6 | | |
| 863 | 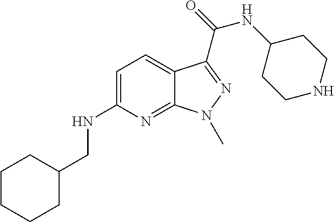 | 1.3 | | |
| 866 | 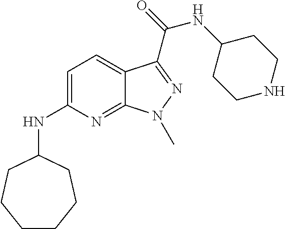 | 1.1 | | |
| 867 | 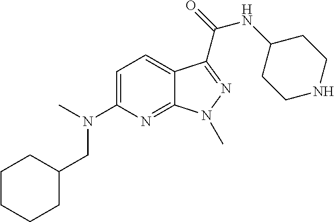 | 0.934 | | |
| 868 | 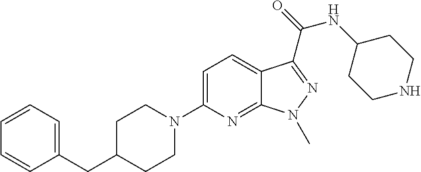 | 2.4 | | |
| 872 | 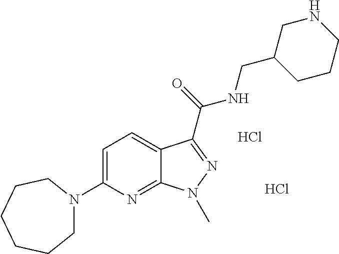 | 8.1 | | |
| 873 | 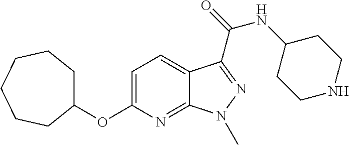 | 4.7 | | |
| 874 | 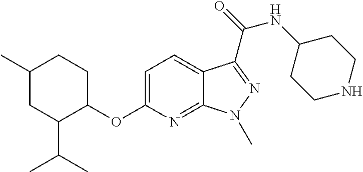 | 1.7 | | |
| 876 | 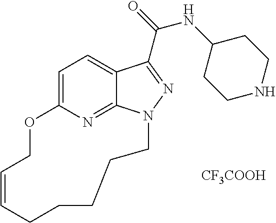 | 7.5 | | |
| 877 | 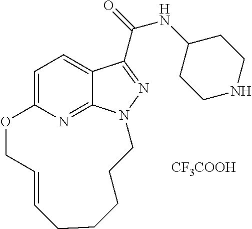 | 3.3 | | |
| 878 | 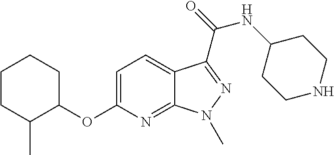 | 2.8 | | |
| 879 | 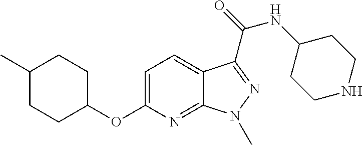 | 3.9 | | |
| 880 | 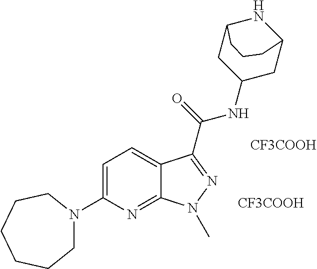 | 8.1 | | |
| 885 | 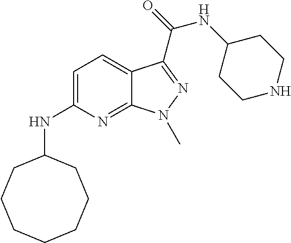 | 0.889 | | |
| 886 | 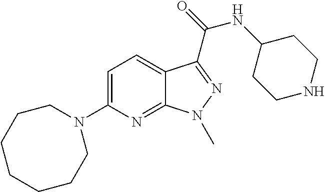 | 0.798 | | |
| 891 | 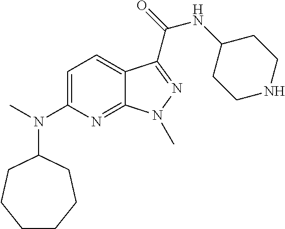 | 1.1 | | |
| 892 | 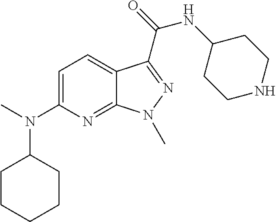 | 1.5 | | |
| 893 | 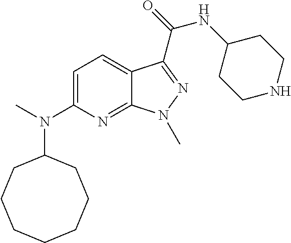 | 1.1 | | |
| 908 | 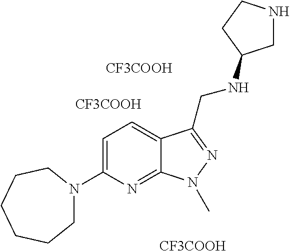 | 8.7 | | |
| 909 | 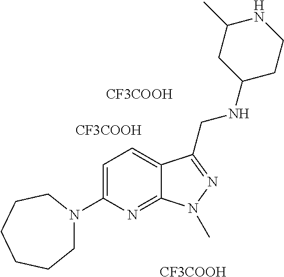 | 4 | | |
| 910 | 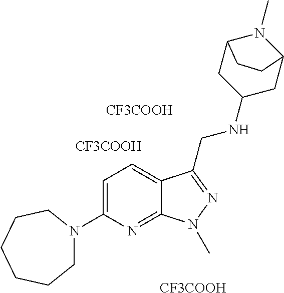 | 3.9 | | |
| 912 | 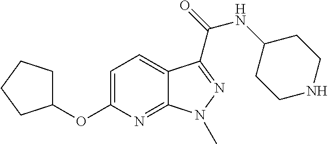 | 6.9 | | |
| 913 | 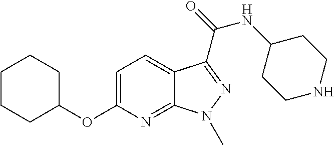 | 4.7 | | |
| 938 | 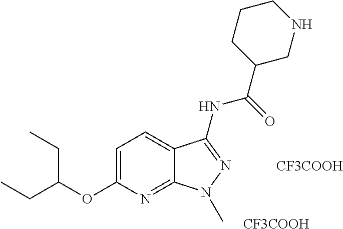 | 8.2 | | |
| 939 | 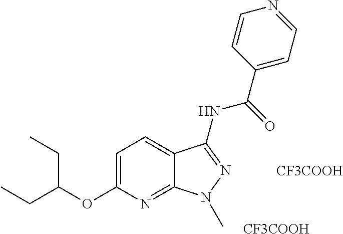 | 4.7 | | |
| 942 | 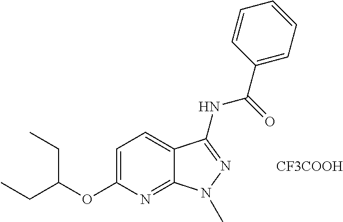 | 8.6 | | |
| 945 | 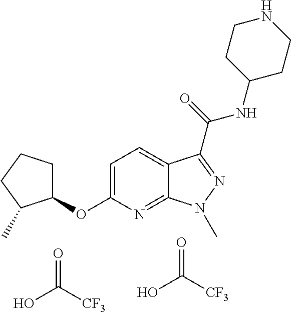 | 2.6 | | |
| 946 | 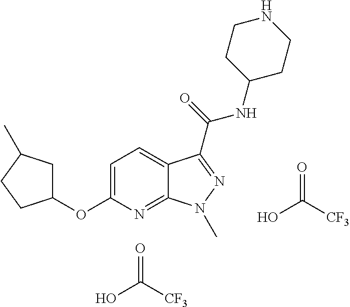 | 3.7 | | |
| 949 | 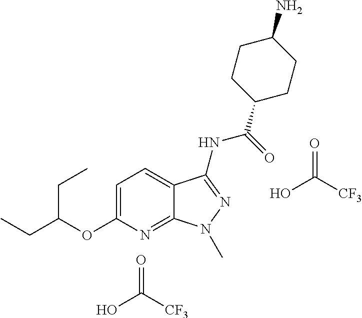 | 5.7 | | |
| 950 | 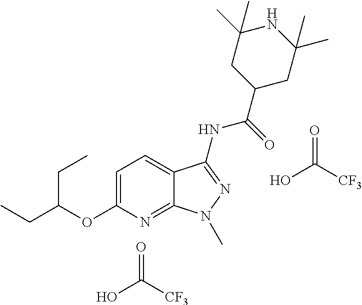 | 7.9 | | |
| 951 | 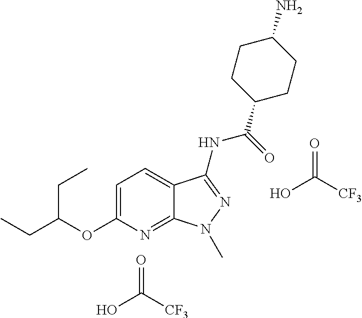 | 7.8 | | |
| 952 | 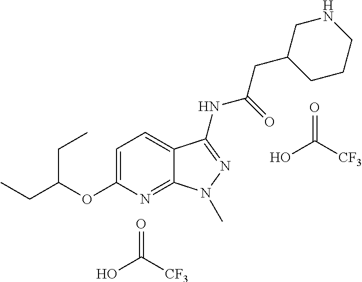 | 10 | | |
| 953 | 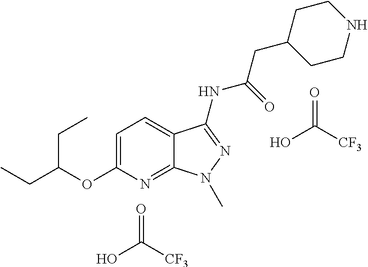 | 6.4 | | |
| 954 | 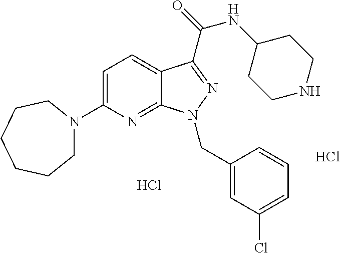 | 7.2 | | |
| 955 | 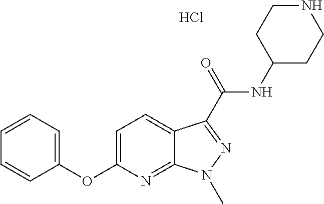 | 3.4 | | |
| 956 | 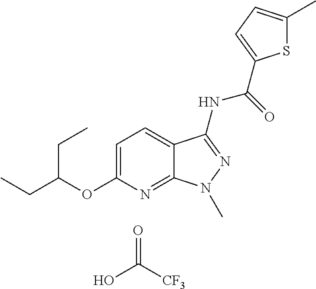 | 21 | | |
| 962 | 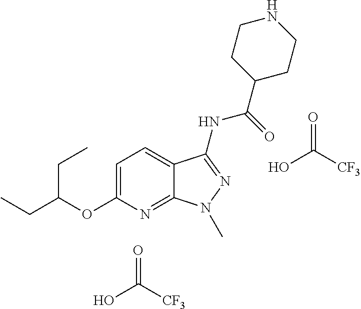 | 4.4 | | |
| 963 | 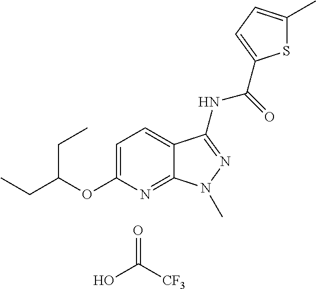 | 2.7 | | |
| 969 | 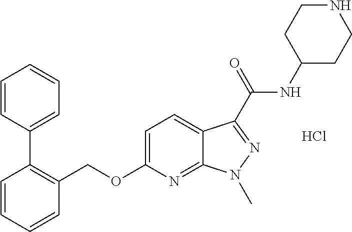 | 1.3 | | |
| 970 | 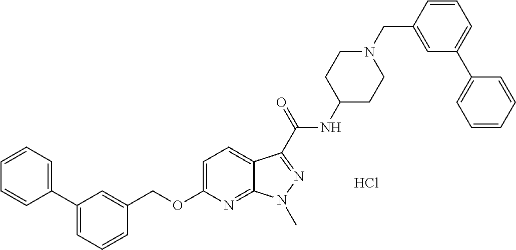 | 8.9 | | |
| 971 | 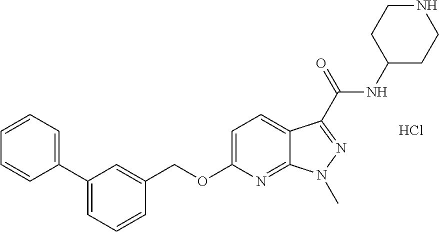 | 8.2 | | |
| 971 | 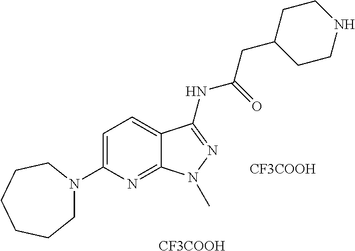 | 8.4 | | |
| 972 | 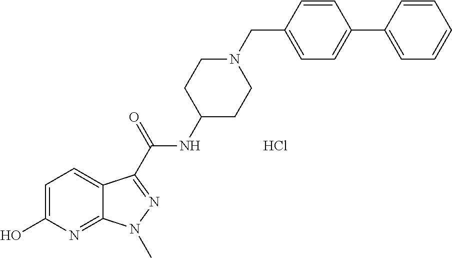 | 3.7 | | |
| 973 | 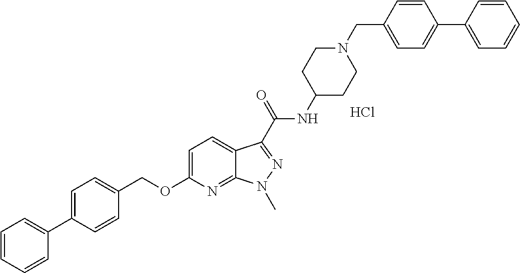 | 5.4 | | |
| 974 | 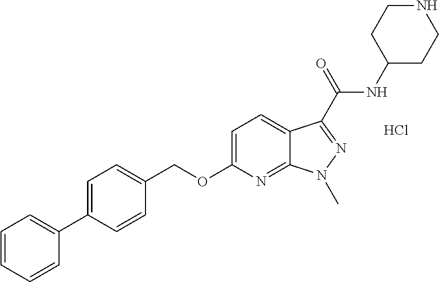 | 4.5 | | |
| 976 | 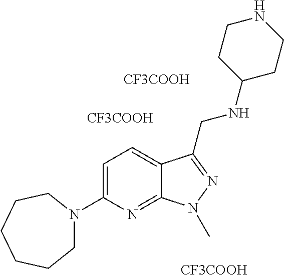 | 4.7 | | |
| 979 | 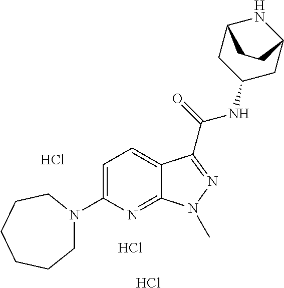 | 4.9 | | |
| 984 | 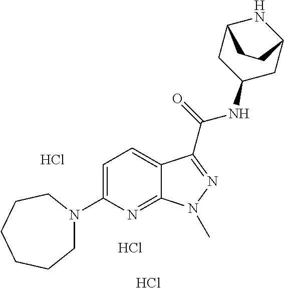 | 2.2 | | |
| 985 | 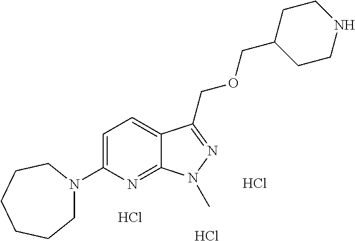 | 7.6 | | |
| 986 | 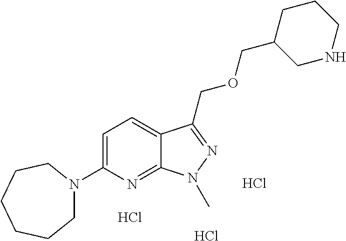 | 7.9 | | |
| 987 | 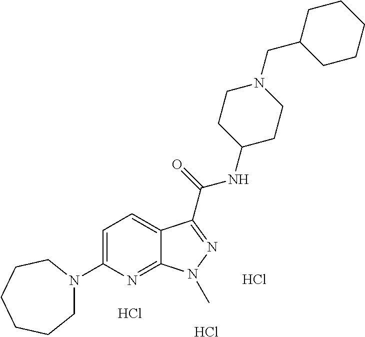 | 0.55 | 0.88 | |
| 988 | 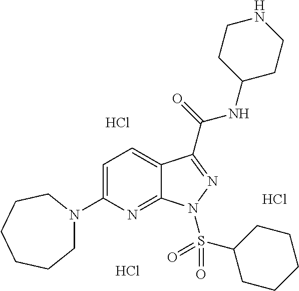 | 5.4 | | |
| 989 | 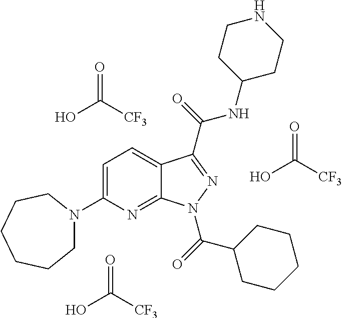 | 2.6 | >10 | Efflux/permeability = 3/5 |
| 991 | 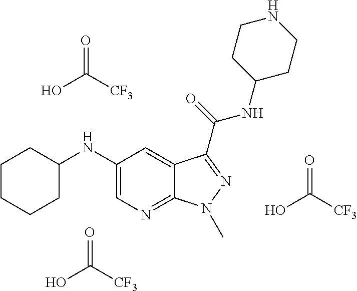 | 9 | | |
| 1026 | 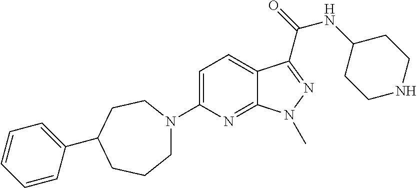 | 1.6 | | |
| 1030 | 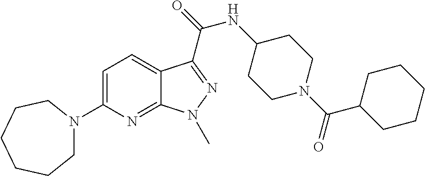 | 4.8 | | |
| 1031 | 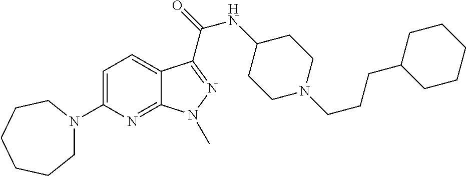 | 0.69 | | |
| 1035 | 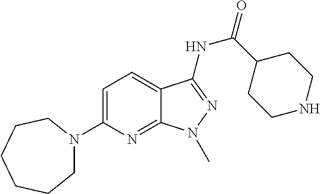 | 5.7 | | |
| 1036 | 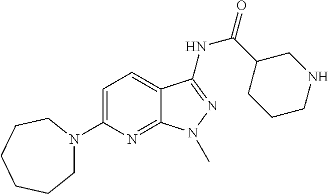 | 8.7 | | |
| 1037 | 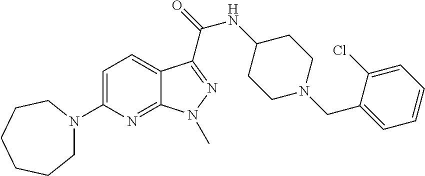 | 3.4 | | |
| 1038 | 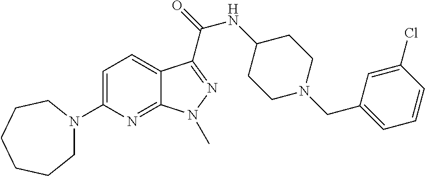 | 3.4 | | |
| 1039 | 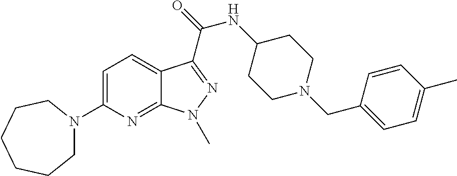 | 2.6 | | |
| 1040 | 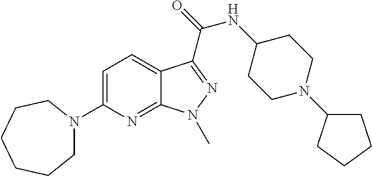 | 2.8 | | |
| 1041 | 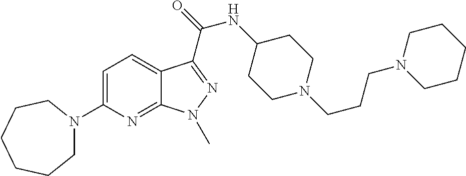 | 1.1 | | |
| 1042 | 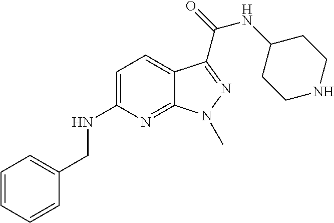 | 3.8 | | |
| 1043 | 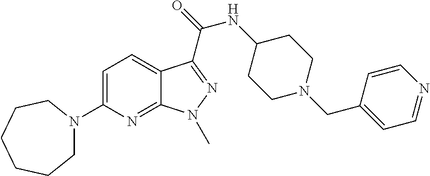 | 1.2 | | |
| 1044 | 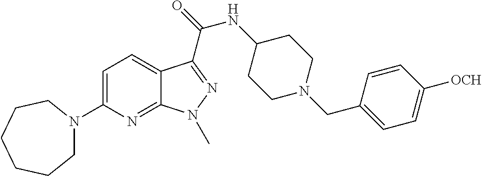 | 0.76 | | |
| 1045 | 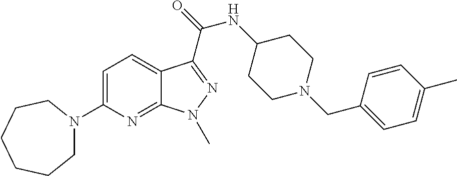 | 1.1 | | |
| 1046 | 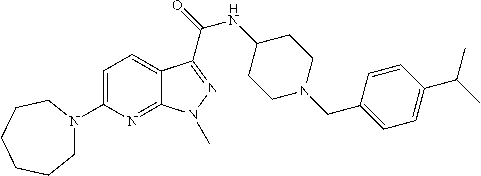 | 1.4 | | |
| 1047 | 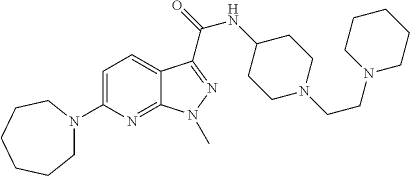 | 0.52 | 9.5 | Efflux/permeability = 7.6/10.5; human liver microsome (HLM, % parent remaining): 78/62/34; rat liver microsome (RLM): 44/18/10; mouse liver microsome (MLM): 56/29/12; CYP (% negative controls) 1A2 = 98; 2C9 = 91; 2C19 = 92; 2D6 = 72; 3A4 = 113 |
| 1049 | 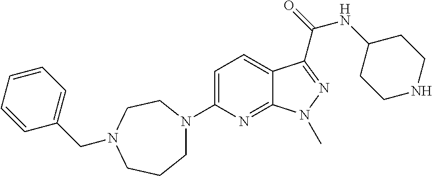 | 6.1 | | |
| 1051 | 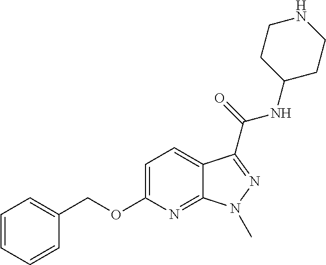 | 2.9 | | |
| 1054 | 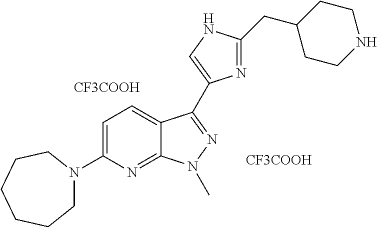 | 8.6 | | |
| 1062 | 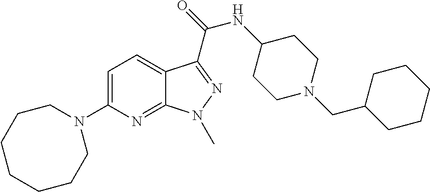 | 0.356 | >10 | Efflux/permeability = 0.6/1.7 |
| 1063 | 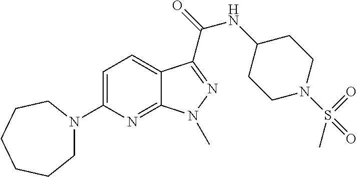 | 3.9 | | Efflux/permeability = 16/50 |
| 1065 | 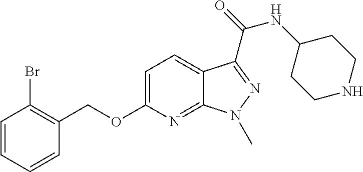 | 3.3 | | |
| 1066 | 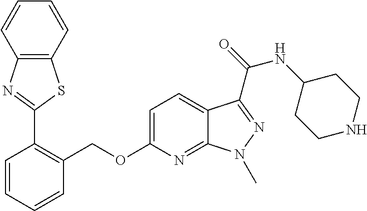 | 2.5 | | |
| 1069 | 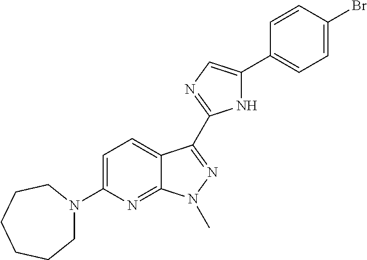 | 8.3 | | |
| 1072 | 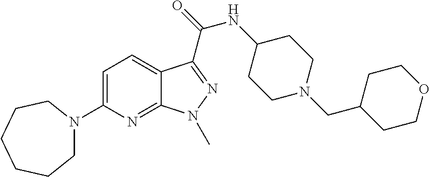 | 2.5 | | Efflux/permeability = 4.6/5.4 |
| 1073 | 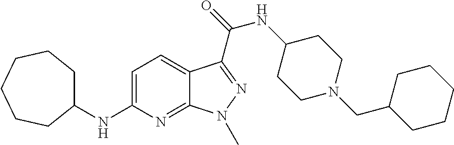 | 0.67 | | |
| 1074 | 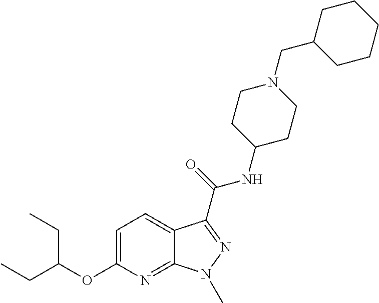 | 0.88 | | Efflux/permeability = 1.6/6.6 |
| 1075 | 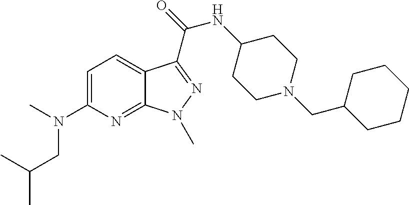 | 0.76 | | |
| 1076 | 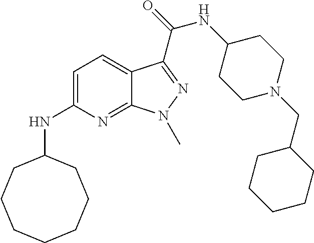 | 0.60 | | |
| 1077 | 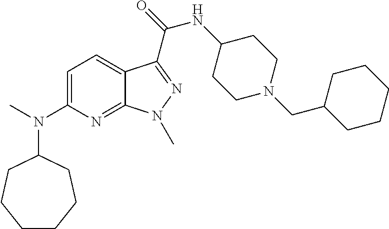 | 1.6 | | |
| 1085 | 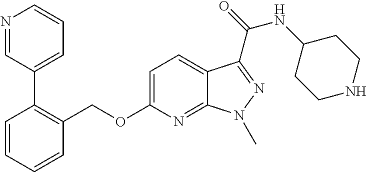 | 2.2 | | |
| 1088 | 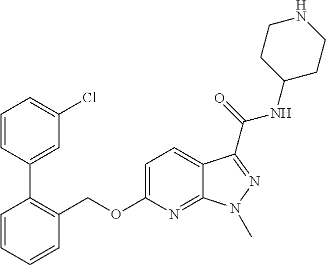 | 1.4 | | |
| 1094 | 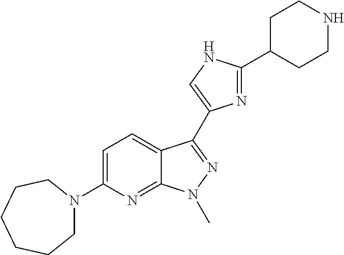 | 6.5 | | |
| 1095 | 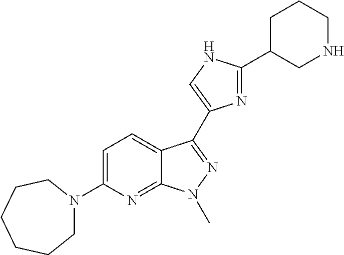 | 3.8 | | |
| 1096 | 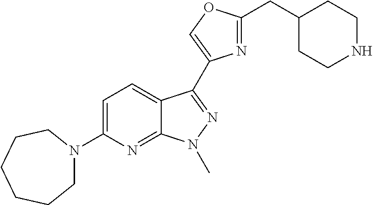 | 2.8 | | |
| 1097 | 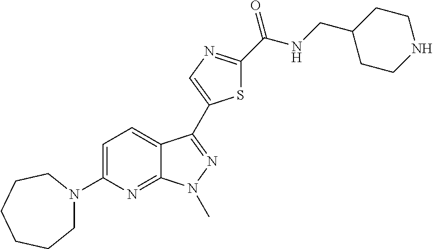 | 3.4 | | |
| 1098 | 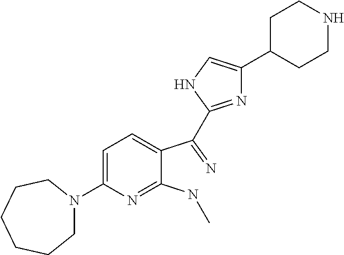 | 1.6 | | |
| 1099 | 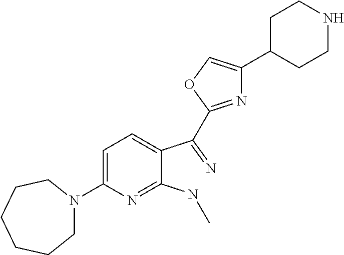 | 1 | | |
| 1100 | 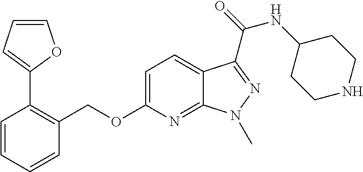 | 1.4 | | |
| 1101 | 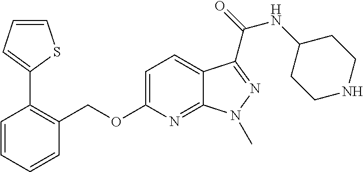 | 0.8 | | |
| 1102 | 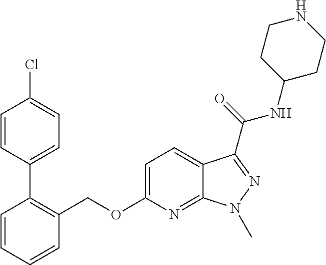 | 0.62 | | |
| 1103 | 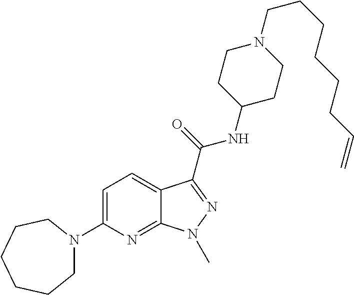 | 0.3 | | |
| 1104 | 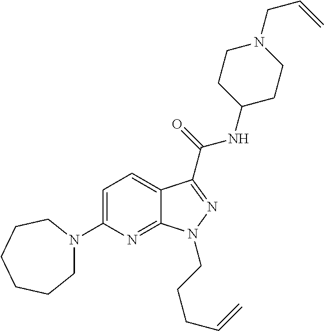 | 2.2 | | |
| 1105 | 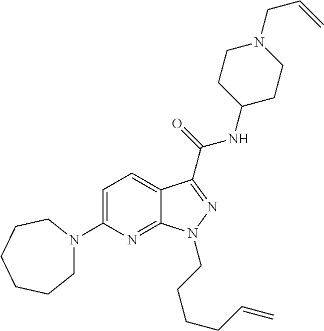 | 7.5 | | |
| 1106 | 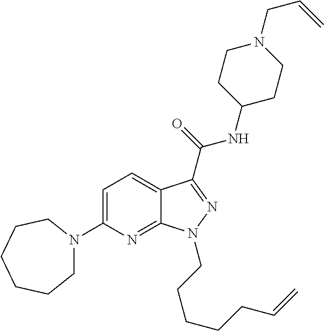 | 8.1 | | |
| 1110 | 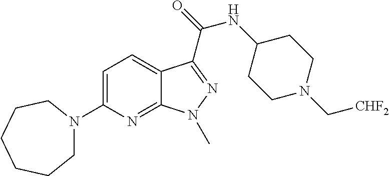 | 1.8 | | Efflux/permeability = 10.1/13.8; human liver microsome (HLM, % parent remaining): 8/0.4/0.4; rat liver microsome (RLM): 10, 0.5, 0.5; mouse liver microsome (MLM): 15, 1, 0.4 |
| 1116 | 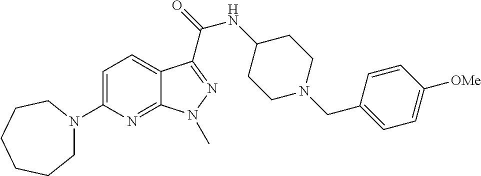 | 1.3 | | Efflux/permeability = 0.3/0.5 |
| 1119 | 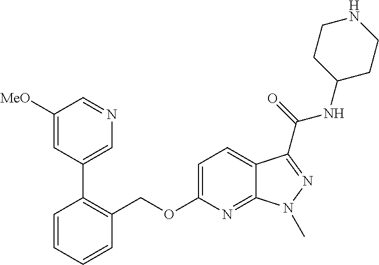 | 1.5 | | |
| 1125 | 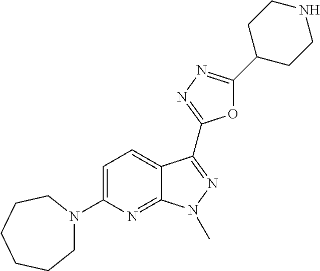 | 0.84 | | |
| 1126 | 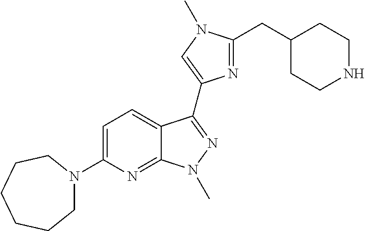 | 7.7 | | |
| 1127 | 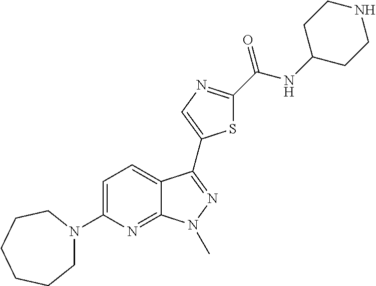 | 2.9 | | |
| 1128 | 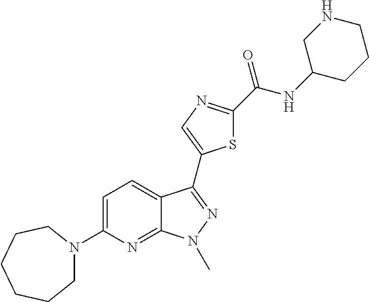 | 0.49 | | |
| 1129 | 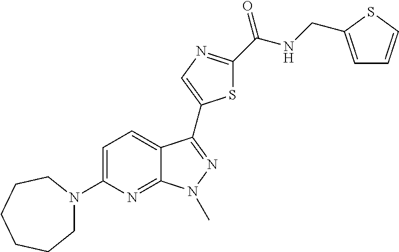 | 0.83 | | Efflux/permeability = 4/6 |
| 1130 | 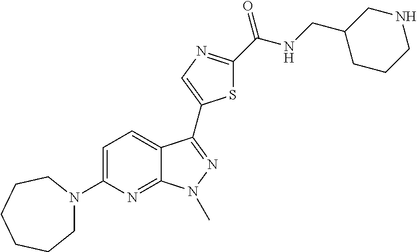 | 0.99 | | |
| 1146 | 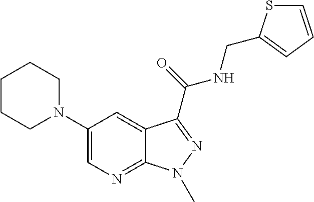 | 7.5 | | Efflux/permeability = 21/22 |
| 1150 | 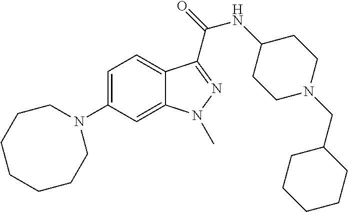 | 0.6 | | Efflux/permeability = 5.7/1.4 |
| 1151 | 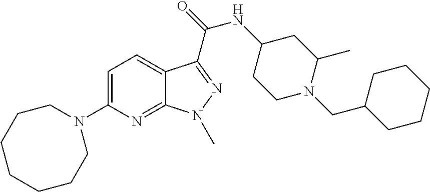 | 1.4 | | |
| 1152 | 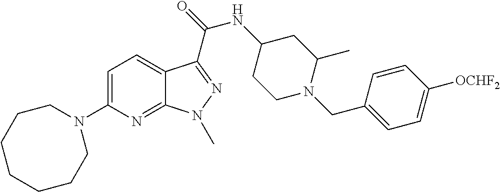 | 6.1 | | |
| 1153 | 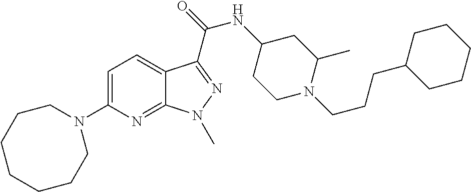 | 1.5 | | |
| 1171 | 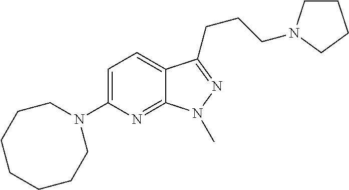 | 1.6 | | Efflux/permeability = 11/17; human liver microsome (HLM, % parent remaining): 67/44/23; rat liver microsome (RLM): 1.2/0.5/0.3; mouse liver microsome (MLM): 22/3/1; CYP (% negative controls) 1A2 = 104; 2C9 = 141; 2C19 = 72; 2D6 = 62; 3A4 = 67 |
| 1172 | 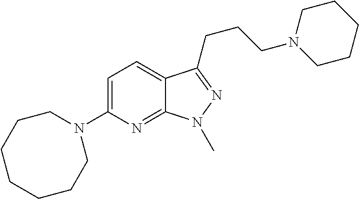 | 1.5 | | |
| 1183 | 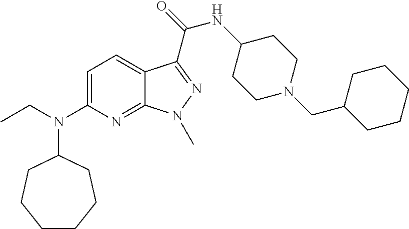 | 0.746 | | |
| 1184 | 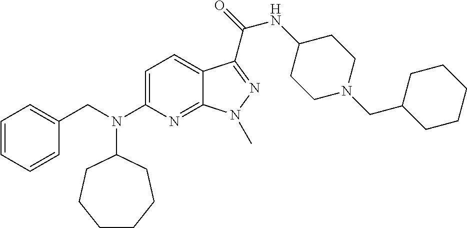 | 0.168 | | |
| 1203 | 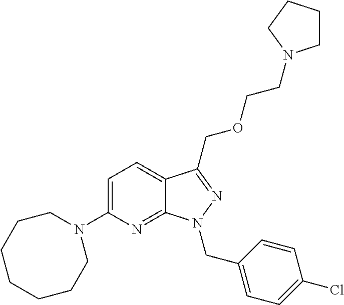 | 3.3 | | Efflux/permeability = 7.3/7.8 |
| 1204 | 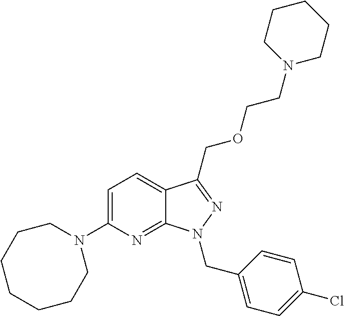 | 5.4 | | |
| 1205 | 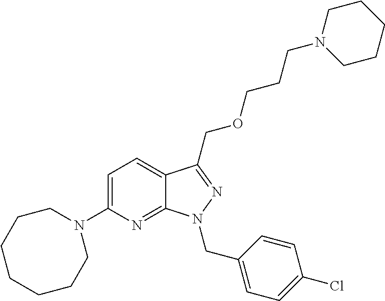 | 4 | | |
| 1222 | 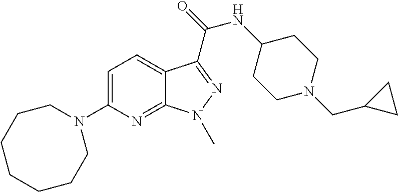 | 3.2 | | |
| 1223 | 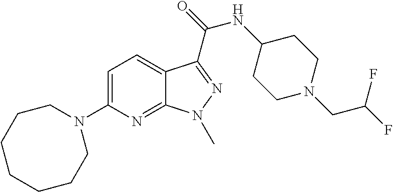 | 2.8 | | |
| 1224 | 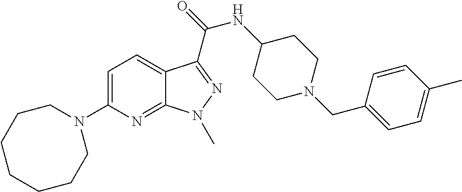 | 2.7 | | |
| 1225 | 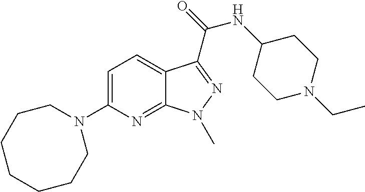 | 1.2 | | Efflux/permeability = 3.2/6.4 |
| 1233 | 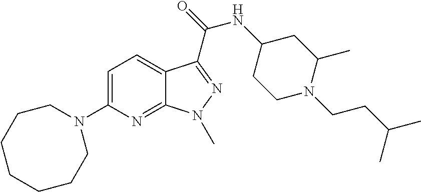 | 5.3 | | |
| 1234 | 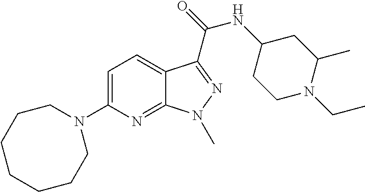 | 1.9 | | |
| 1235 | 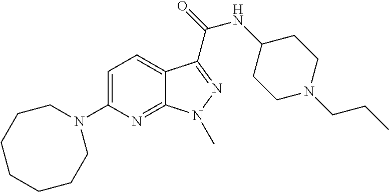 | 3.4 | | |
| 1236 | 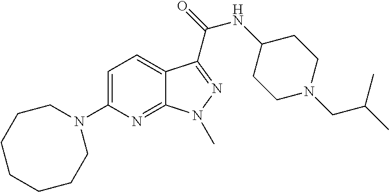 | 6.3 | | |
| 1248 | 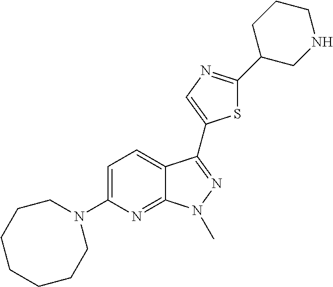 | 4.1 | | |
| 1250 | 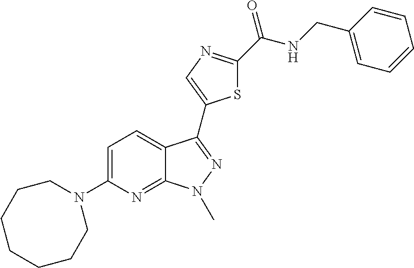 | 1.4 | | |
| 1251 | 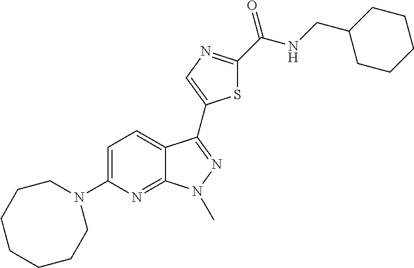 | 1 | | |
| 1254 | 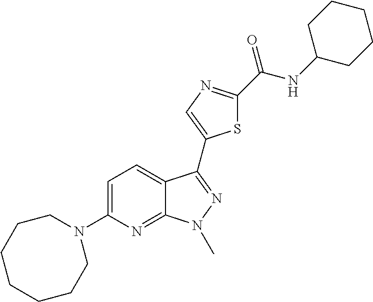 | 1.4 | | |
| 1255 | 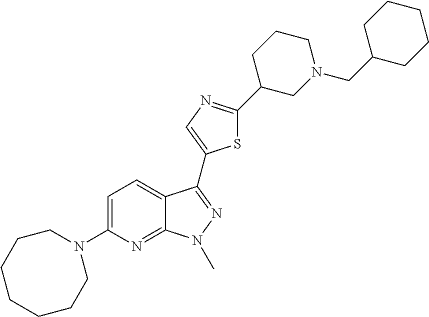 | 3 | | |
| 1257 | 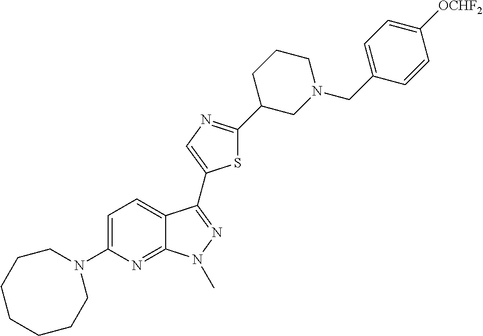 | 2.4 | | |
| 1261 | 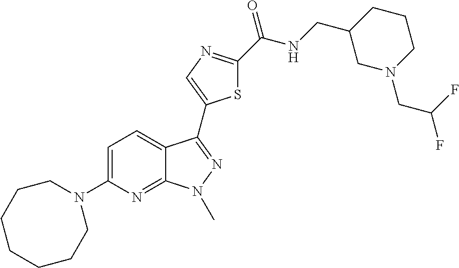 | 1.8 | | Efflux/permeability = 0/0 |
| 1264 | 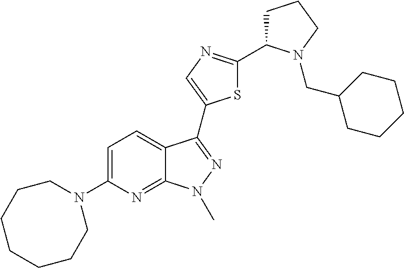 | 2.4 | | |
| 1268 | 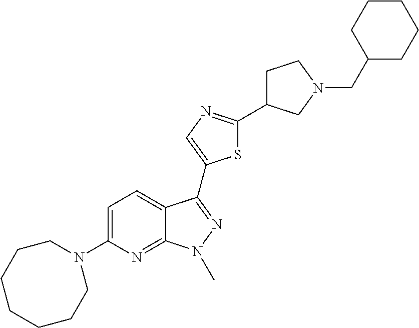 | 0.58 | | |
| 1269 | 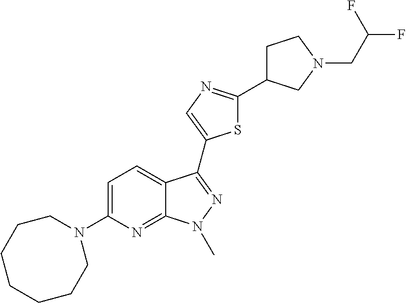 | 4.6 | | |
| 1271 | 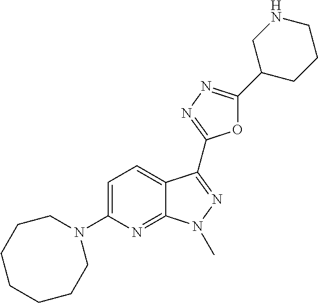 | 3.2 | | |
| 1272 | 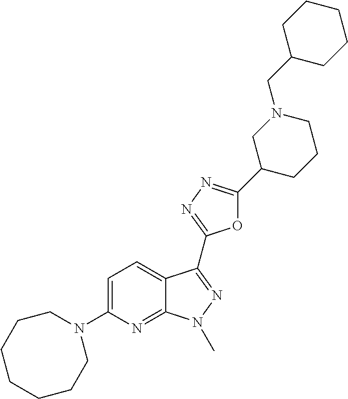 | 0.94 | | |
| 1273 | 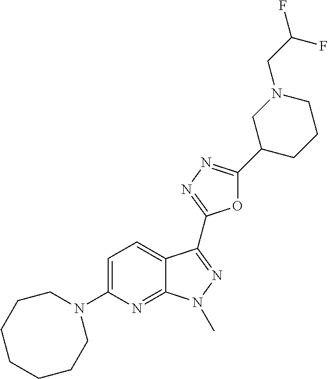 | 4.8 | | |
| 1296 | 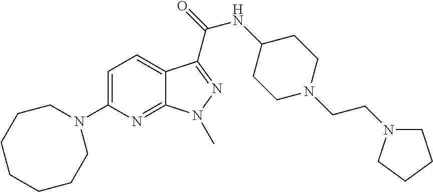 | 0.50 | | |
| 1299 | 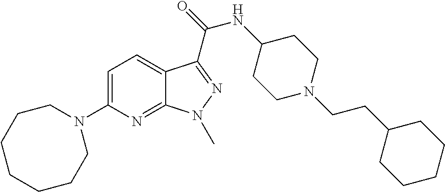 | 0.31 | | |
| 1300 | 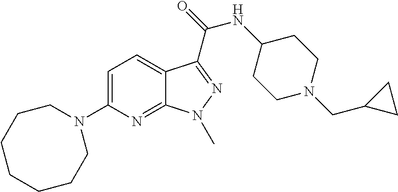 | 2.7 | | |
| 1301 | 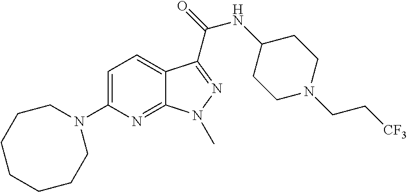 | 5.1 | | |
| 1303 | 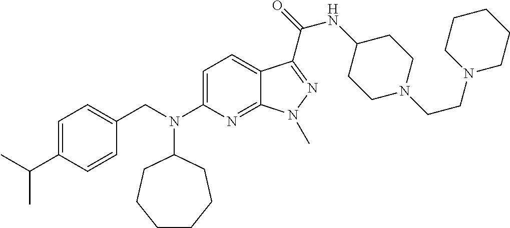 | 0.66 | | |
| 1304 | 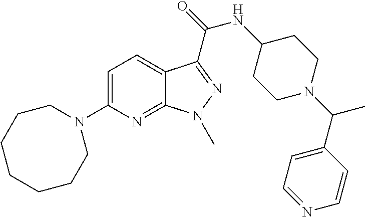 | 1.4 | | |
| 1305 | 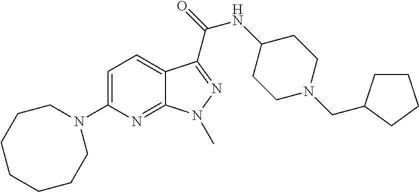 | 0.49 | | |
| 1306 | 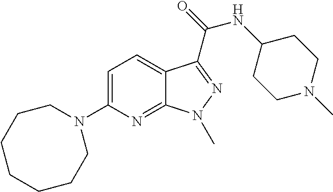 | 0.36 | | Efflux/permeability = 4.4/9.1 |
| 1307 | 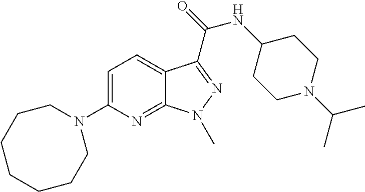 | 1 | | Efflux/permeability = 4.2/9.6 |
| 1308 | 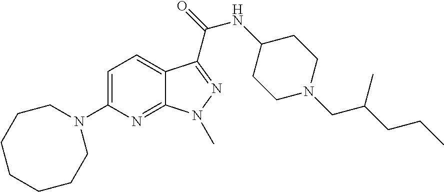 | 0.48 | | Efflux/permeability = 0.2/0.4 |
| 1310 | 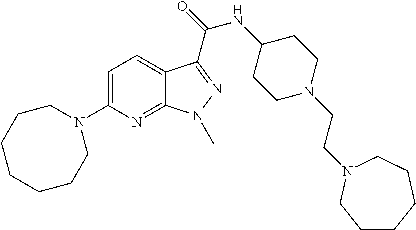 | 0.23 | | Efflux/permeability = 2.1/1.3 |
| 1311 | 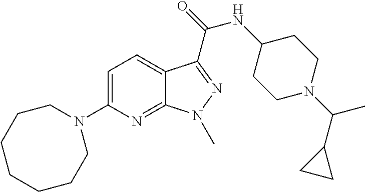 | 0.97 | | |
| 1312 | 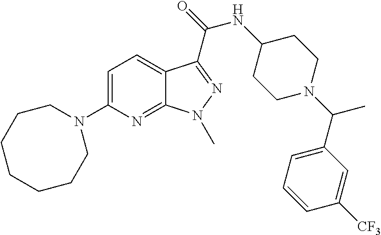 | 2.7 | | |
| 1316 | 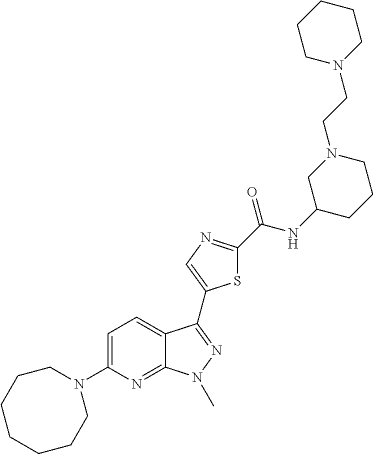 | 0.55 | | |
| 1322 | 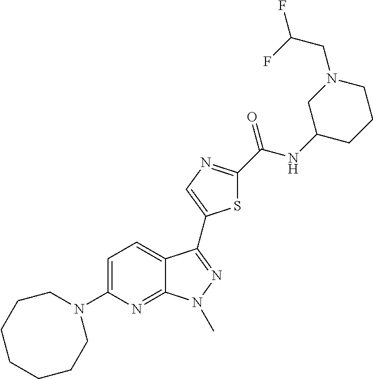 | 1.1 | | |
| 1323 | 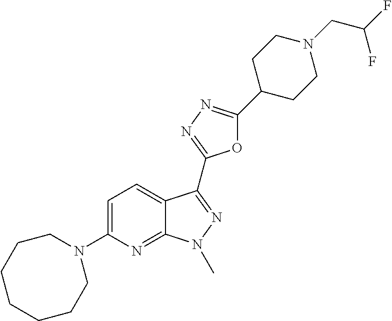 | 4.4 | | |
| 1324 | 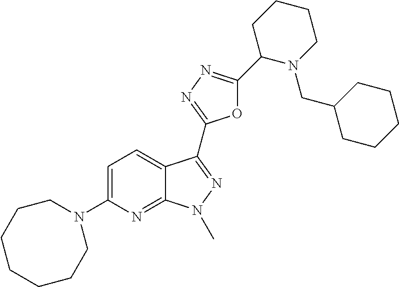 | 0.66 | | |
| 1326 | 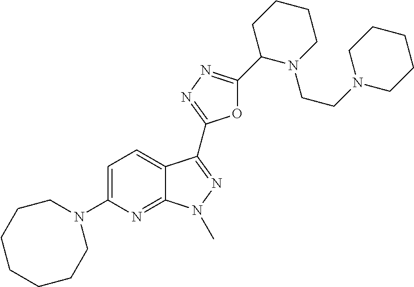 | 4.8 | | |
| 1349 | 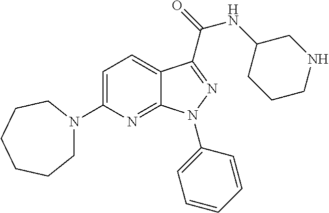 | 5 | | |
| 1350 | 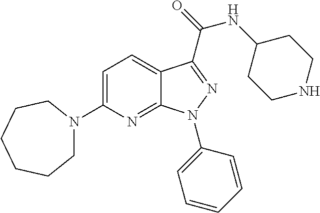 | 3.8 | | |
| 1363 | 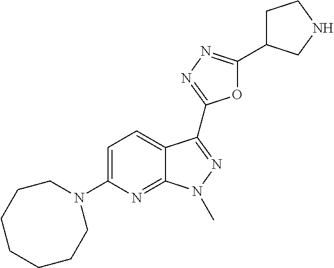 | 2.7 | | |
| 1364 | 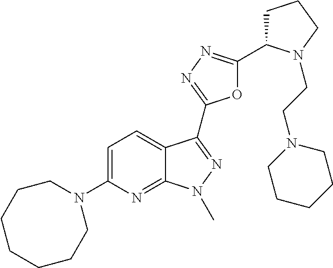 | 2.2 | | |
| 1365 | 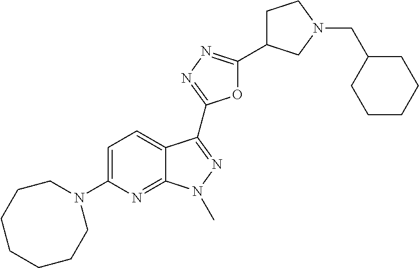 | 2.6 | | |
| 1366 | 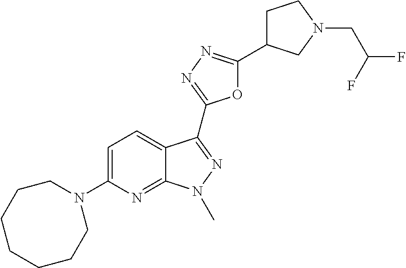 | 4 | | |
| 1368 | 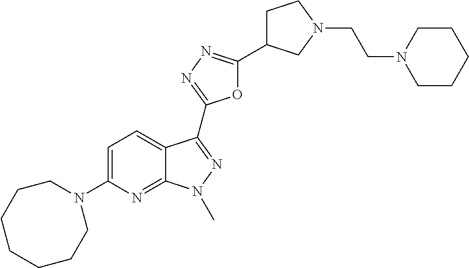 | 4.8 | | |
| 1373 | 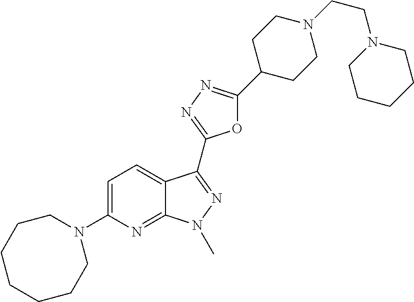 | 0.63 | | |
| 1378 | 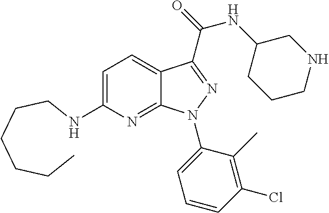 | 2.6 | | |
| 1424 | 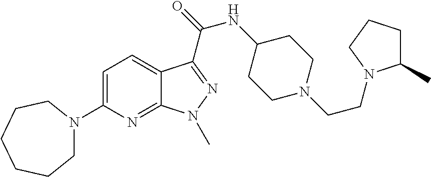 | 0.79 | | |
| 1425 | 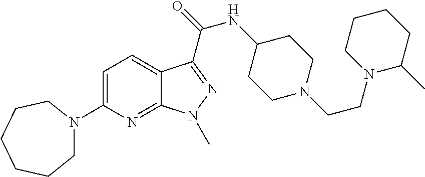 | 0.5 | | Efflux/permeability = 4/3 |
| 1426 | 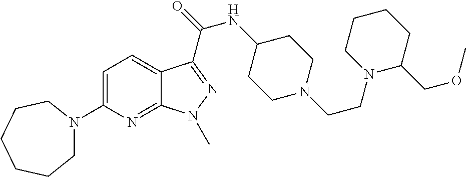 | 0.45 | | |
| 1452 | 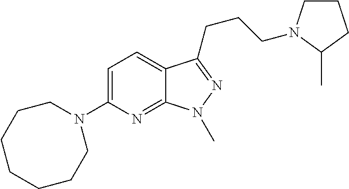 | 1.2 | | Human liver microsome (HLM, % parent remaining): 32/13/3; rat liver microsome (RLM): 2/0.8/0.5; mouse liver microsome (MLM): 1.7/0.9/0.3 |
| 1453 | 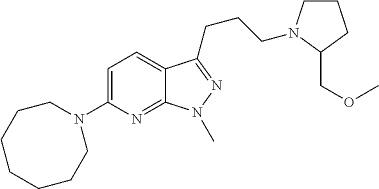 | 2.8 | | |
| 1454 | 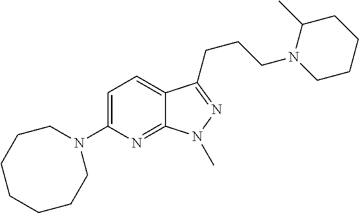 | 1.6 | | |
| 1456 | 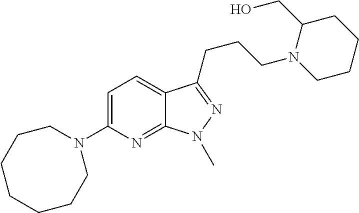 | 1.5 | | Efflux/permeability = 15/15; (HLM, % parent remaining): 28/6/0.7; rat liver microsome (RLM): 3.9/0.4/0.2; mouse liver microsome (MLM): 1.7/0.9/0.3 |
| 1458 | 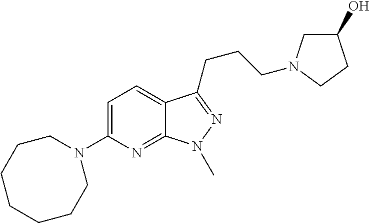 | 3.2 | | |
| 1460 | 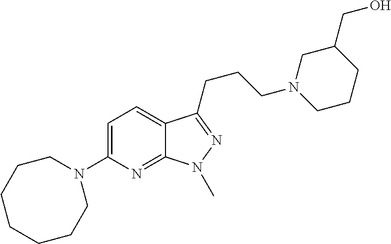 | 2.7 | | |
| 1461 | 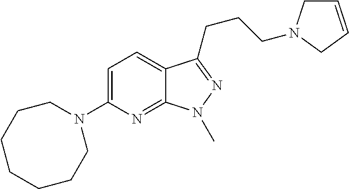 | 2.6 | | |
| 1462 | 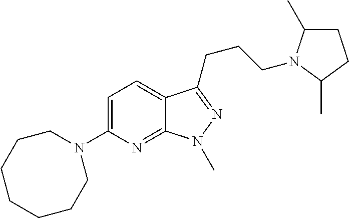 | 1.2 | | |
| 1464 | 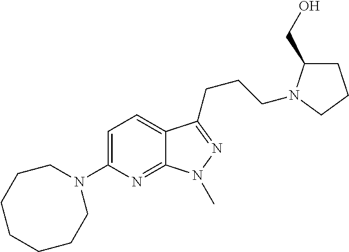 | 2.0 | | |
| 1465 | 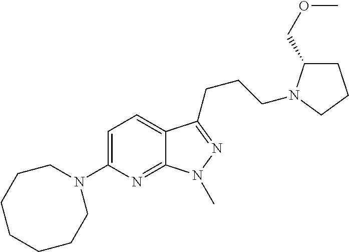 | 4.7 | | |
| 1468 | 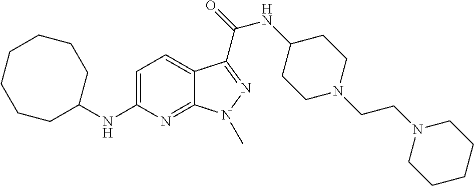 | 0.33 | | |
| 1469 | 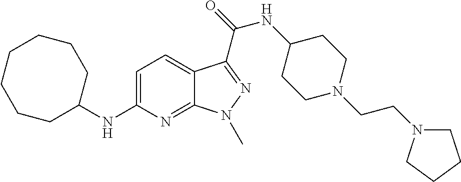 | 0.25 | | Efflux/permeability = 2.9/0.9 |
| 1471 | 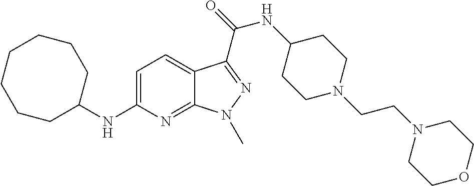 | 0.87 | | Efflux/permeability = 9.6/10.9 |
| 1472 | 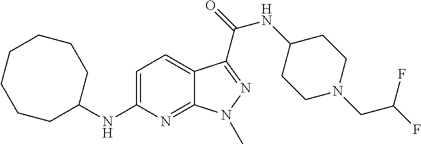 | 1.4 | | |
| 1473 | 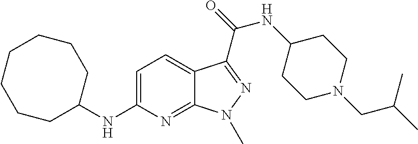 | 0.89 | | |
| 1474 | 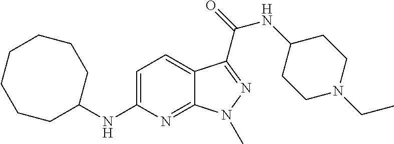 | 0.48 | | |
| 1475 | 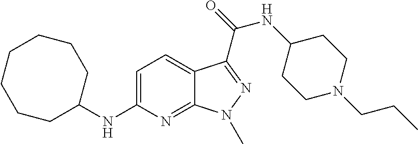 | 0.83 | | Efflux/permeability = 6.3/5.9 |
| 1476 | 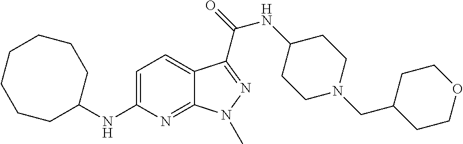 | 0.57 | | |
| 1477 | 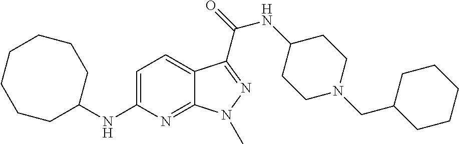 | 0.53 | | |
| 1478 | 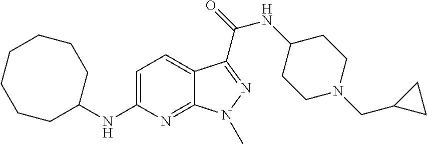 | 0.56 | | |
| 1479 | 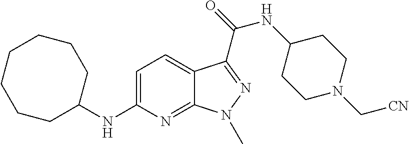 | 0.92 | | |
| 1486 | 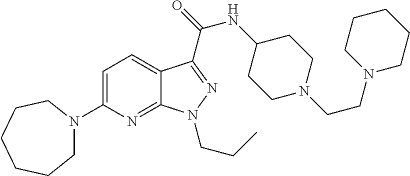 | 0.76 | | |
| 1487 | 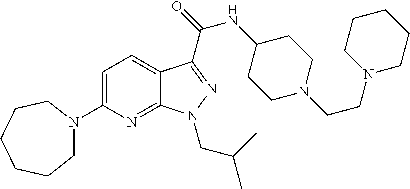 | 0.83 | | |
| 1488 | 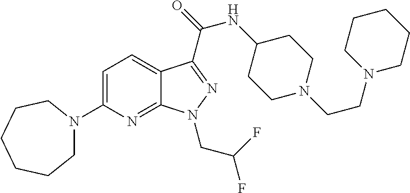 | 0.64 | | Efflux/permeability = 3.5/3.3 |
| 1489 | 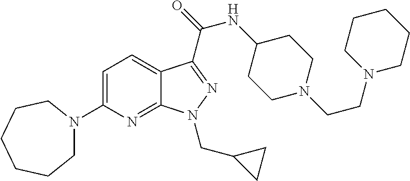 | 0.55 | | |
| 1495 | 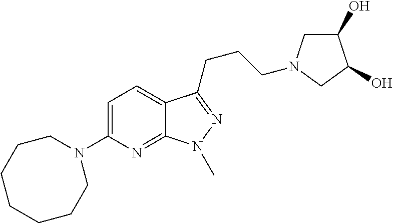 | 4.7 | | |
| 1499 | 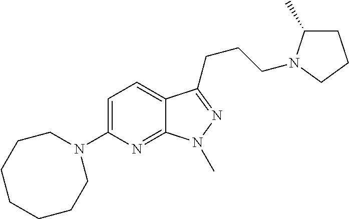 | 4 | | |
| 1540 | 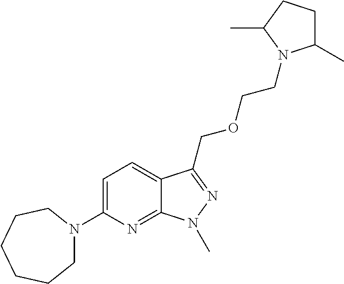 | 2.7 | | Human liver microsome (HLM, % parent remaining): 17/9/4; rat liver microsome (RLM): 17/2/4; mouse liver microsome (MLM): 34/18/10 |
| 1549 | 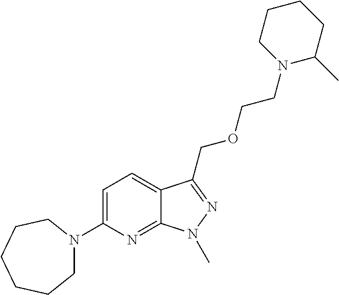 | 4 | | Human liver microsome (HLM, % parent remaining): 16/11/13; rat liver microsome (RLM): 52/31/17; mouse liver microsome (MLM): 27/17/13 |
| 1556 | 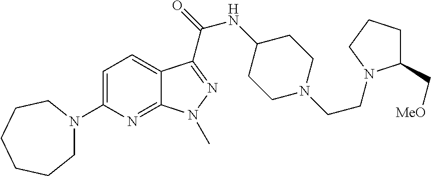 | 2.9 | | |
| 1557 | 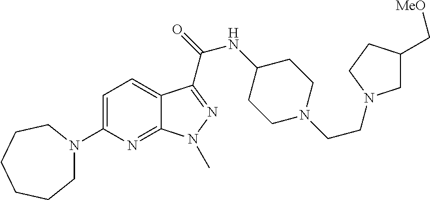 | 2.1 | | |
| 1558 | 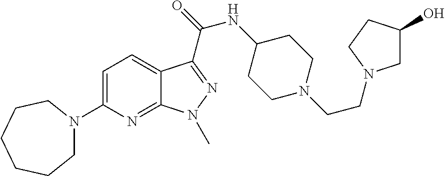 | 3.4 | | |
| 1559 | 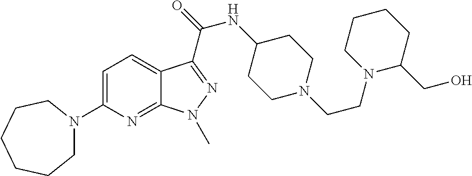 | 1.8 | | Human liver microsome (HLM, % parent remaining): 65/32/7; rat liver microsome (RLM): 57/31/8; mouse liver microsome (MLM): 54/27/5 |
| 1560 | 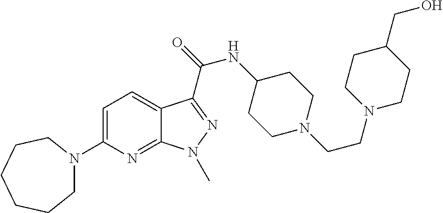 | 0.86 | | Efflux/permeability = 13.1/2 |
| 1561 | 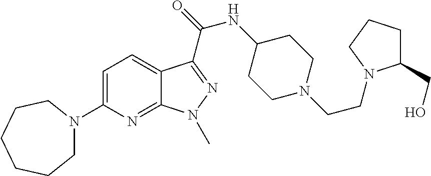 | 3.8 | | |
| 1562 | 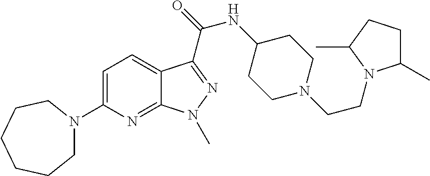 | 1.9 | | Efflux/permeability = 6.3/6.7 |
| 1581 | 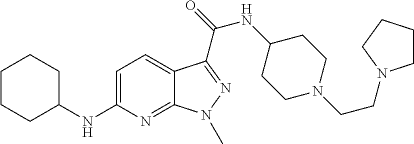 | 3.3 | | |
| 1594 | 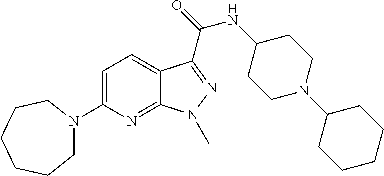 | 1.8 | | |
| 1595 | 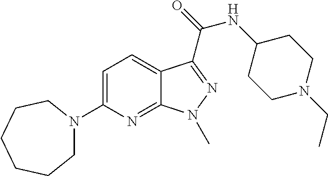 | 0.74 | 1.1 | Efflux/permeability = 14.4/15.3; HLM (% parent remaining): 73, 45, 14; RLM: 67, 46, 8; MLM: 67, 50, 17 |
| 1596 | 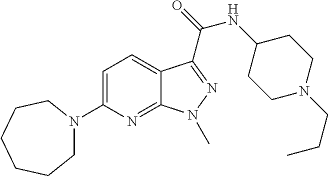 | 1.4 | | |
| 1597 | 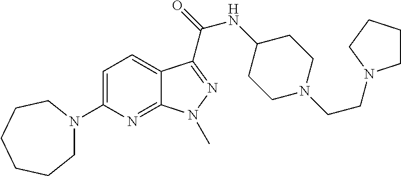 | 0.77 | 9.1 | Efflux/permeability = 6.5/9.2; HLM (% parent remaining): 81, 74, 58; RLM: 57, 25, 14; MLM: 67, 32, 14 |
| 1598 | 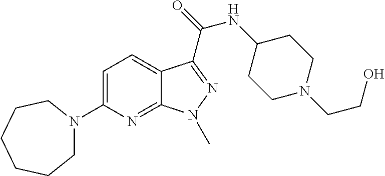 | 2.7 | | |
| 1604 | 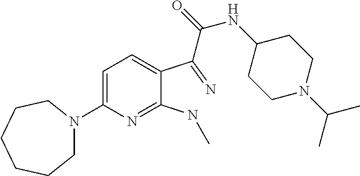 | 2.2 | | Efflux/permeability = 8.6/12.2; HLM (% parent remaining): 71, 63, 28; RLM: 58, 41, 14; MLM: 57, 40, 14 |
| 1609 | 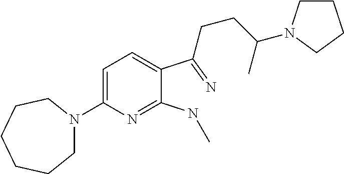 | | | |
| 1619 | 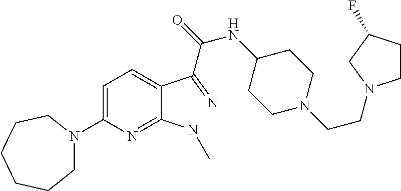 | | | |
| 1620 | 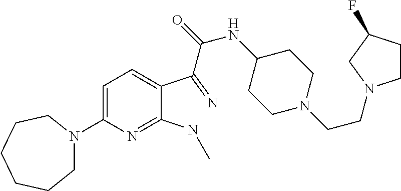 | | | |
| 1621 | 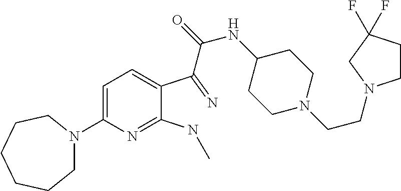 | | | |
| 1622 | 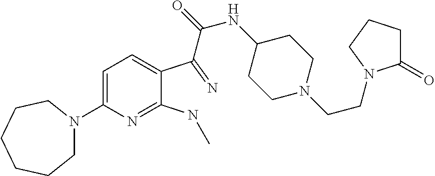 | | | |
| 1632 | 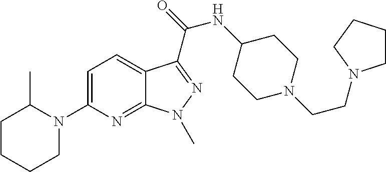 | | | |
| 1633 | 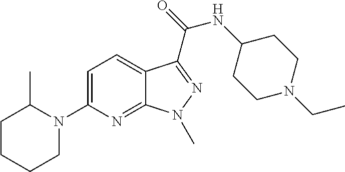 | | | |
| 1634 | 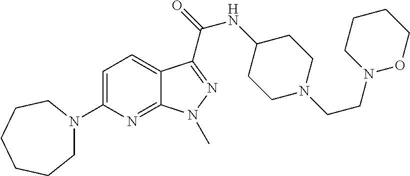 | | | |
| 1635 | 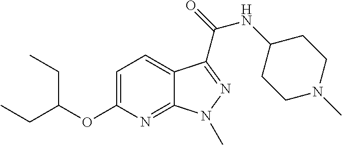 | | | |
| 1636 | 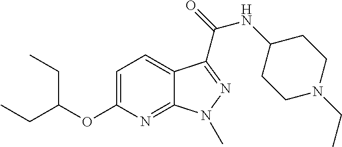 | | | |
| 1637 | 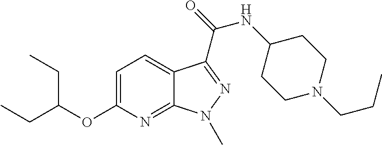 | | | |
| 1638 | 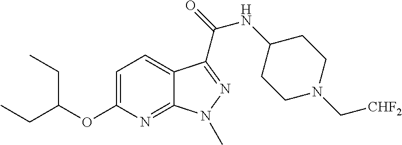 | | | |
| 1639 | 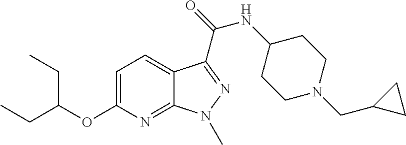 | | | |
| 1640 | 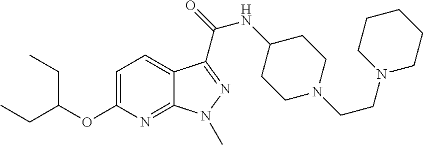 | | | |
| 1641 | 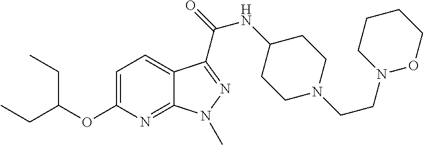 | | | |
| 1642 | 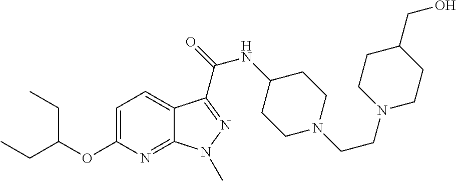 | | | |
| 1643 | 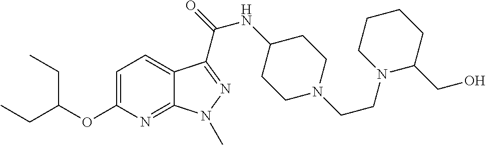 | | | |
| 1644 | 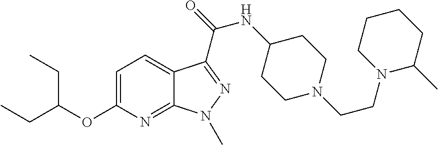 | | | |
| 1645 | 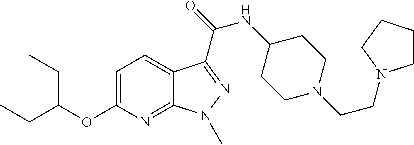 | | | |
| 1646 | 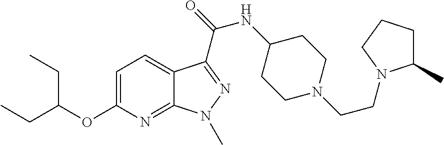 | | | |
| 1647 | 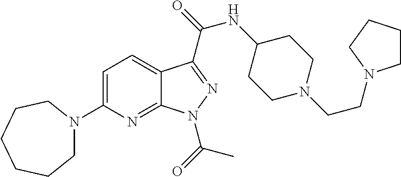 | | | |
| 1648 | 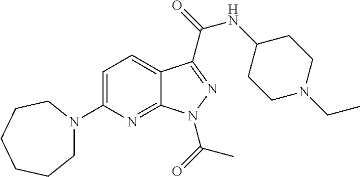 | | | |
| 1649 | 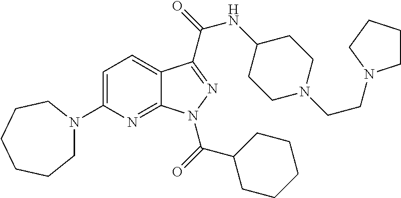 | | | |
| 1650 | 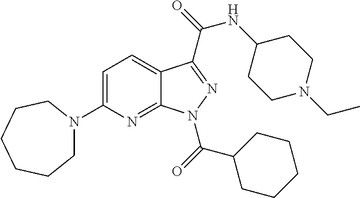 | | | |
| 1651 | 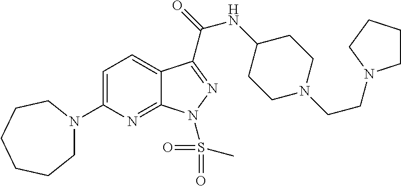 | | | |
| 1652 | 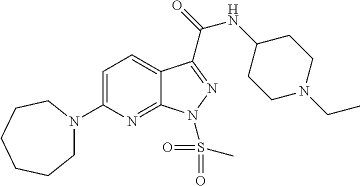 | | | |
| 1653 | 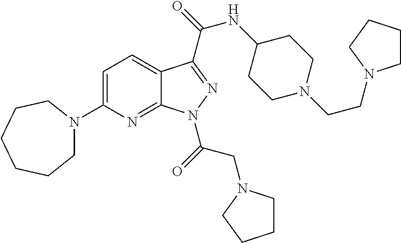 | | | |
| 1654 | 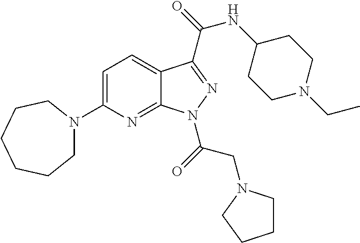 | | | |
| 1655 | 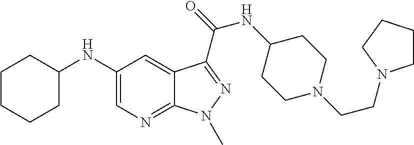 | | | |
| 1656 | 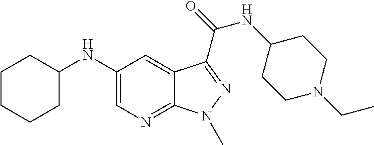 | | | |
| 1657 | 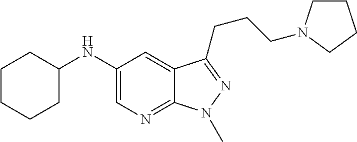 | | | |
| 1658 | 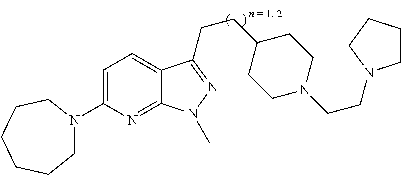 | | | |
| 1659 | 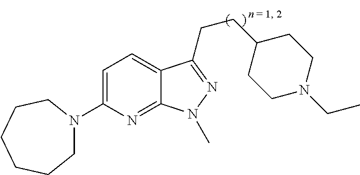 | | | |

In one embodiment, the present invention provides the compounds shown below, which are expected to have EC₅₀ values less than or equal to 25 micromolar in the HCV 2a infectious clone assay described in the examples below, but have (or are expected to have) EC₅₀ values greater than 25 micromolar in the HCV 1b replicon assay described in the examples below.

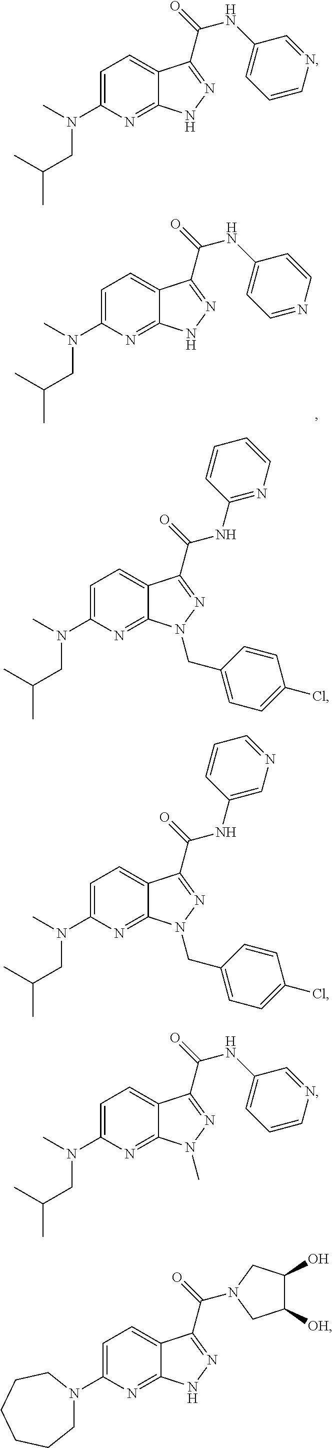

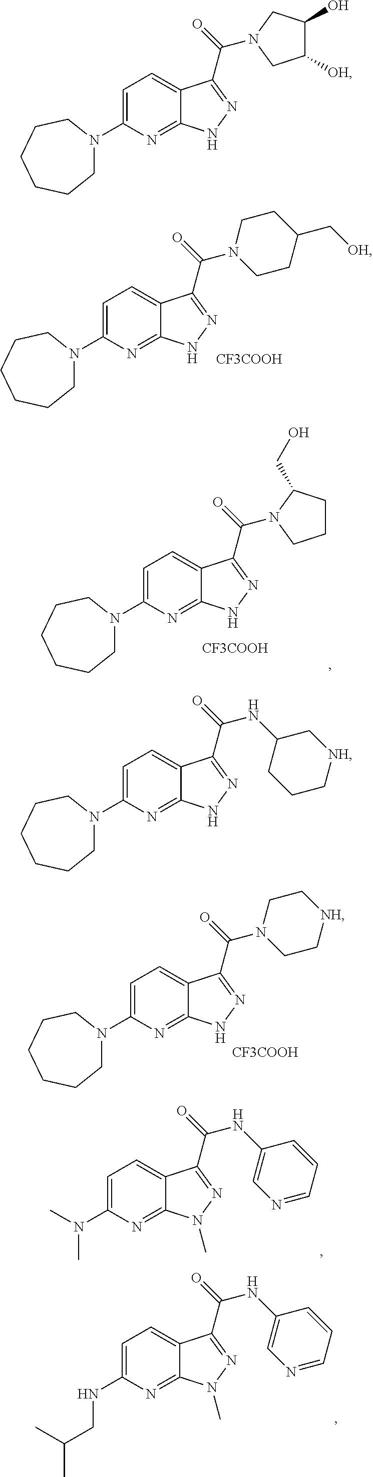

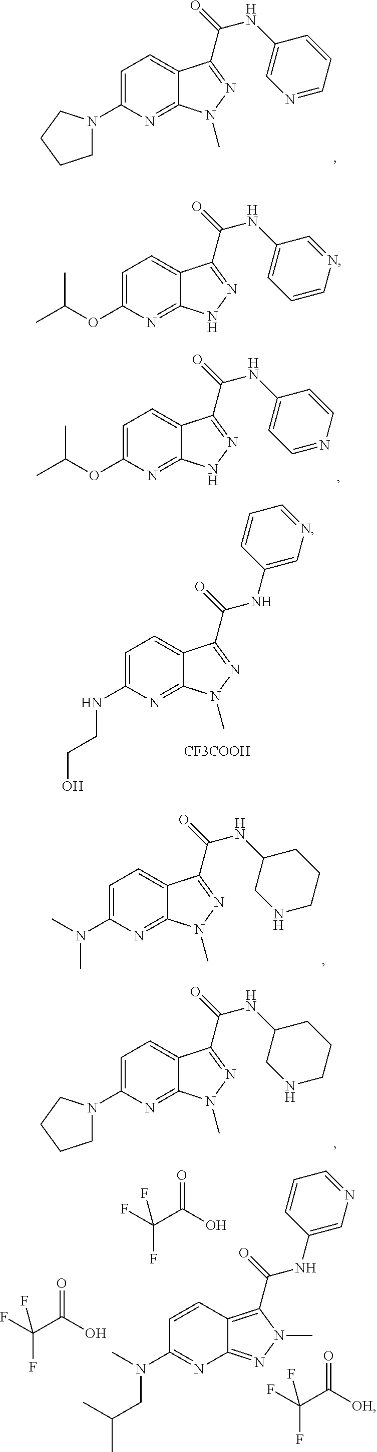

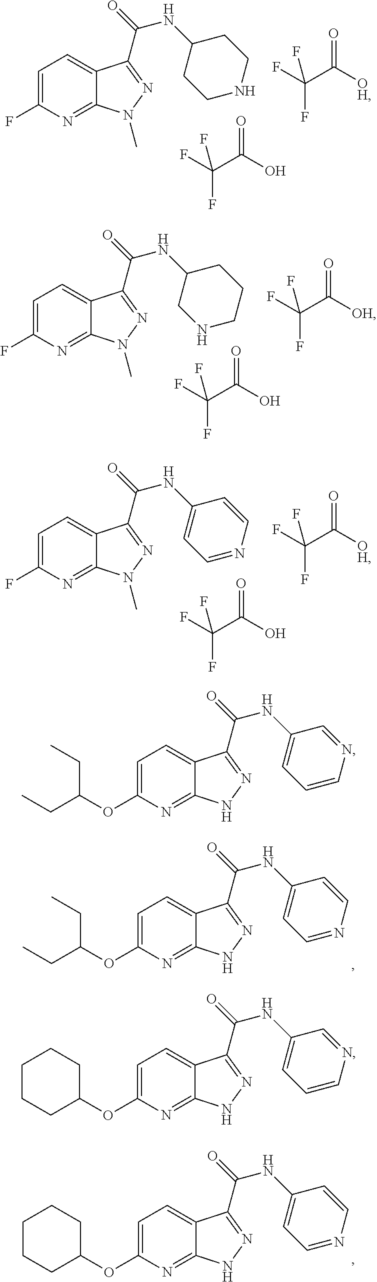

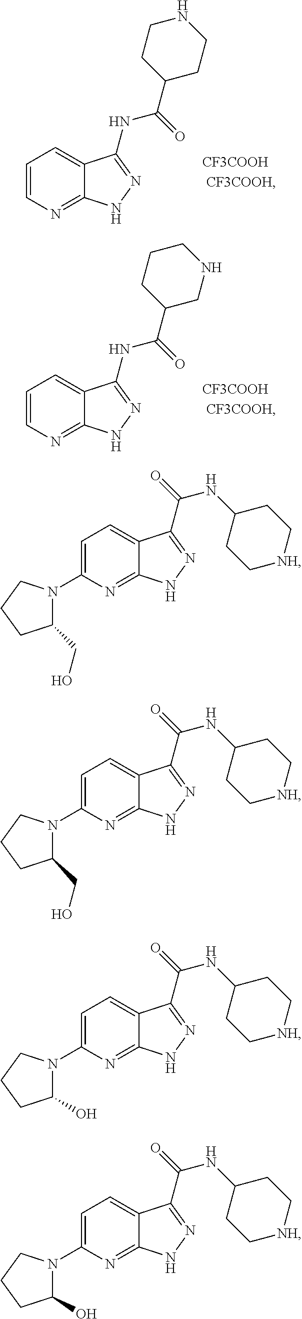

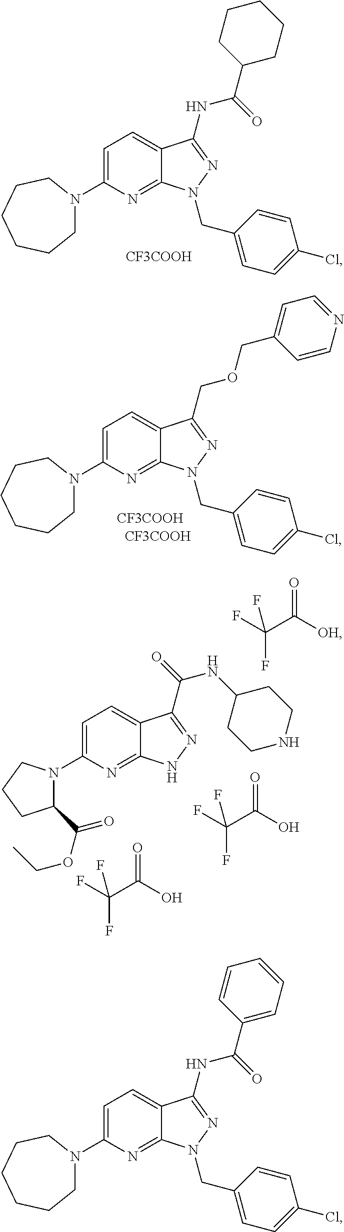

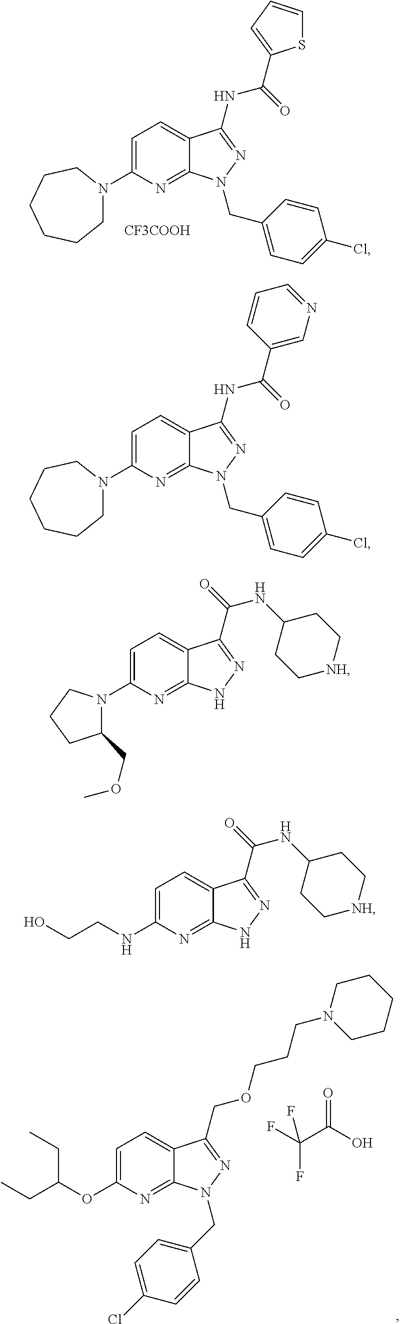

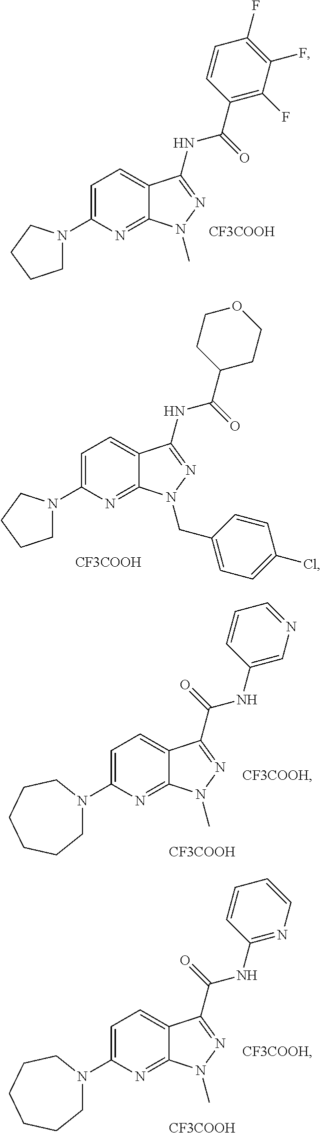

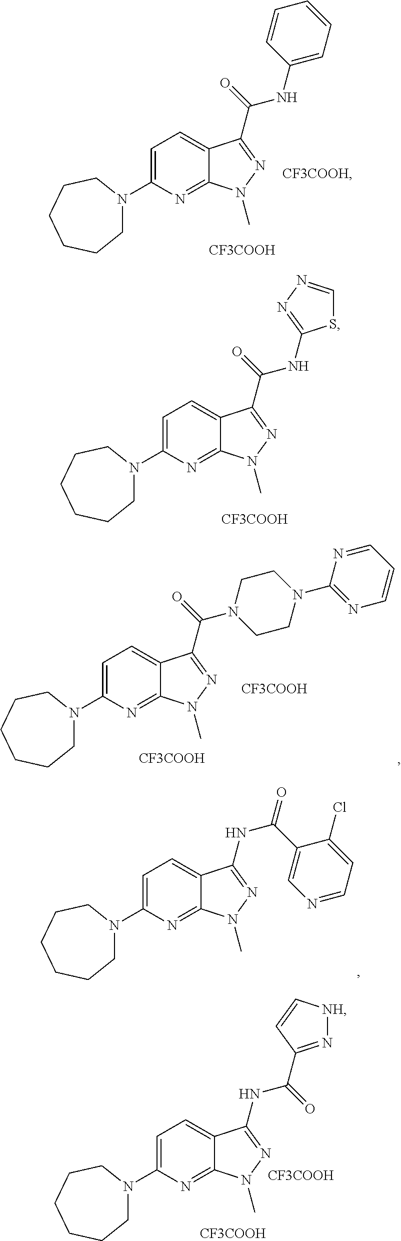

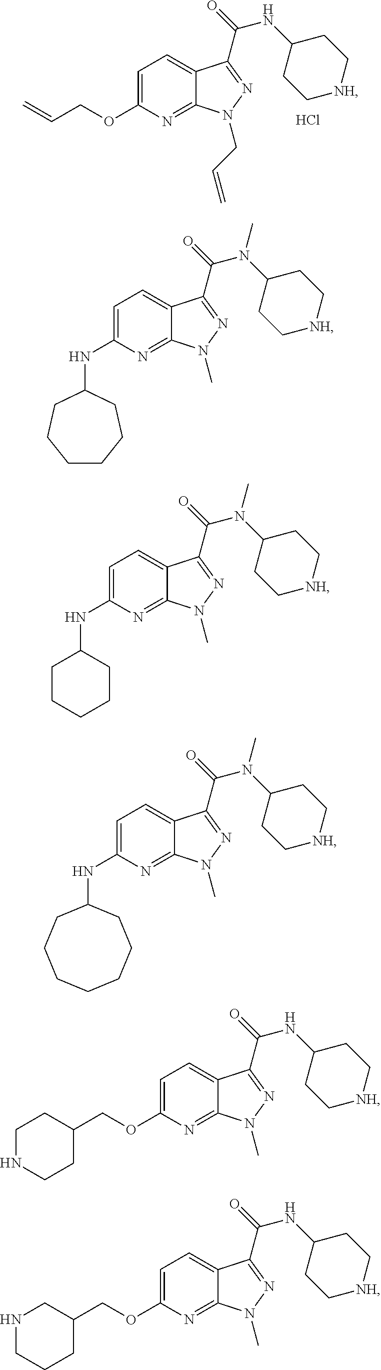

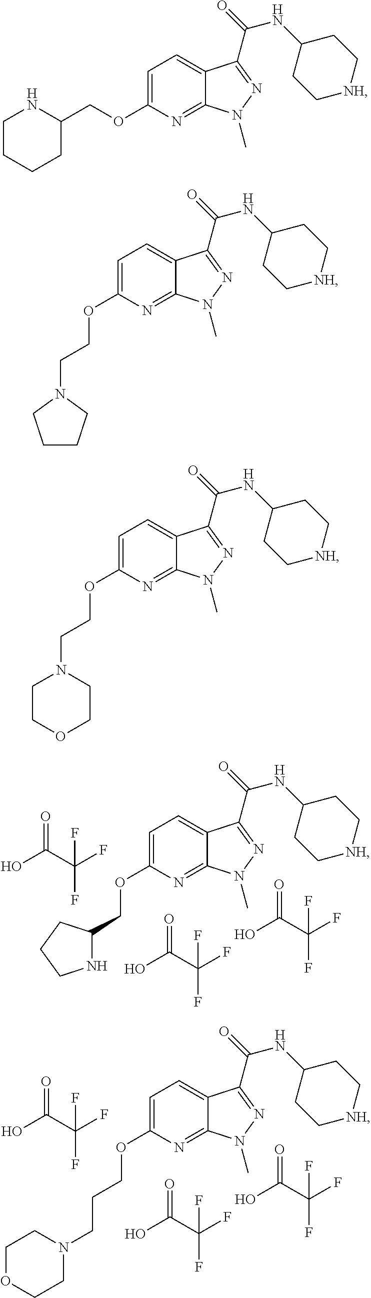

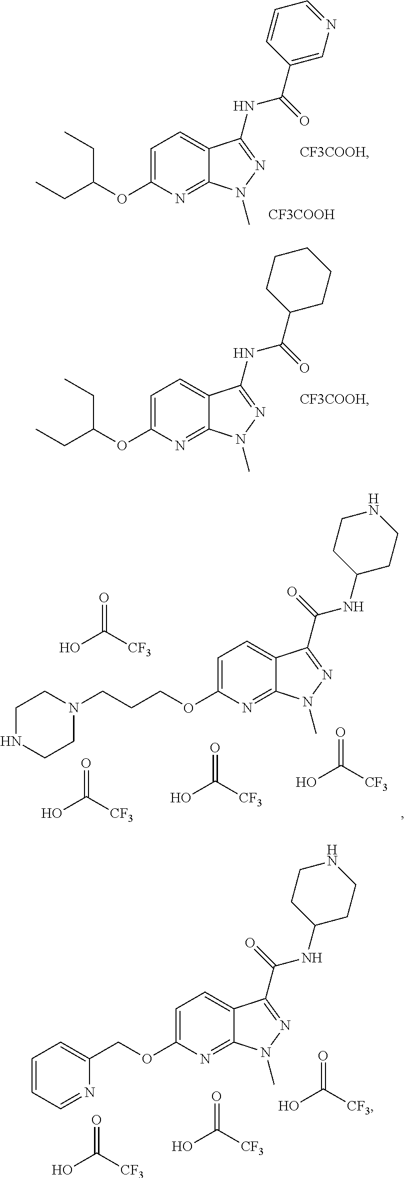

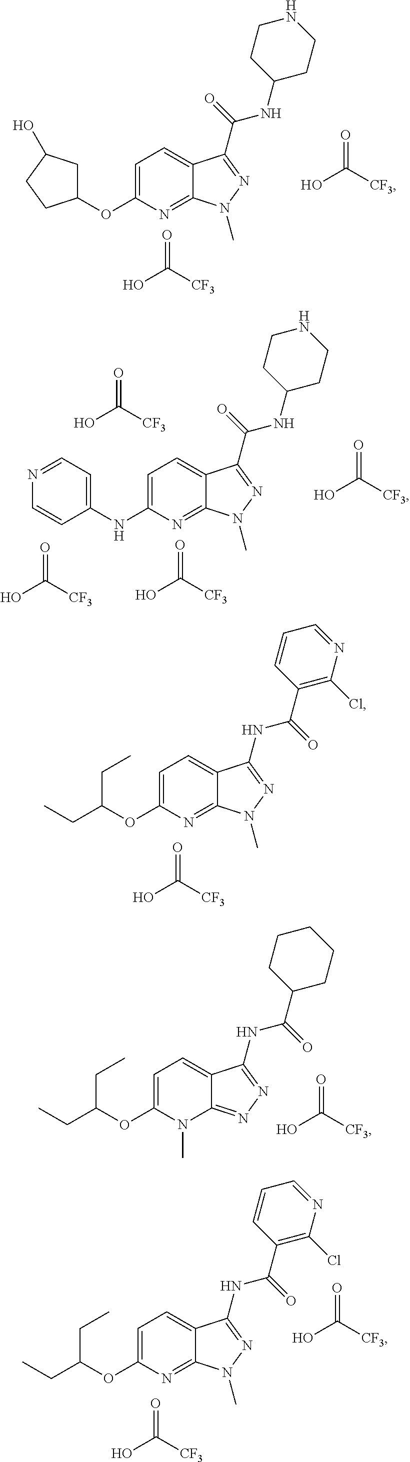

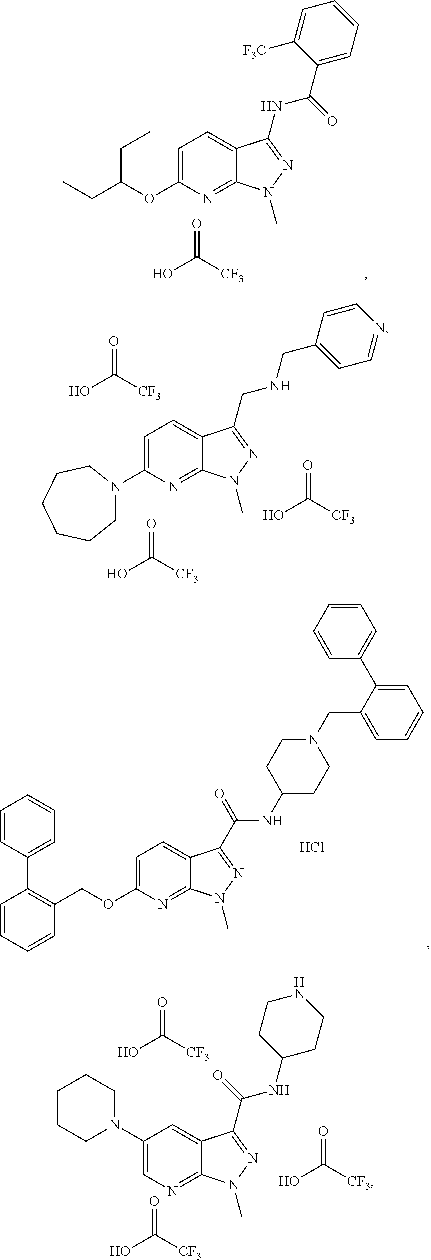

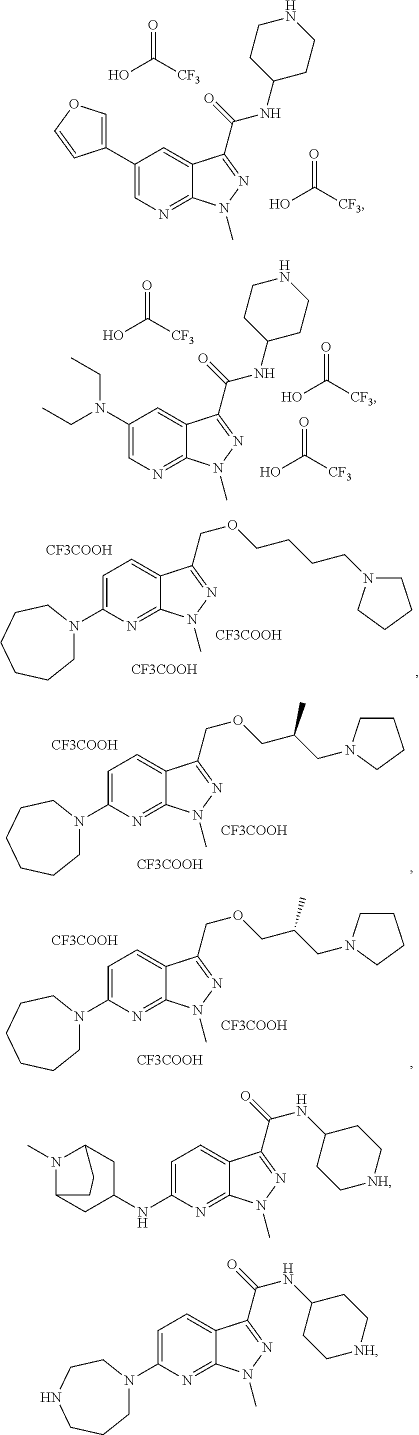

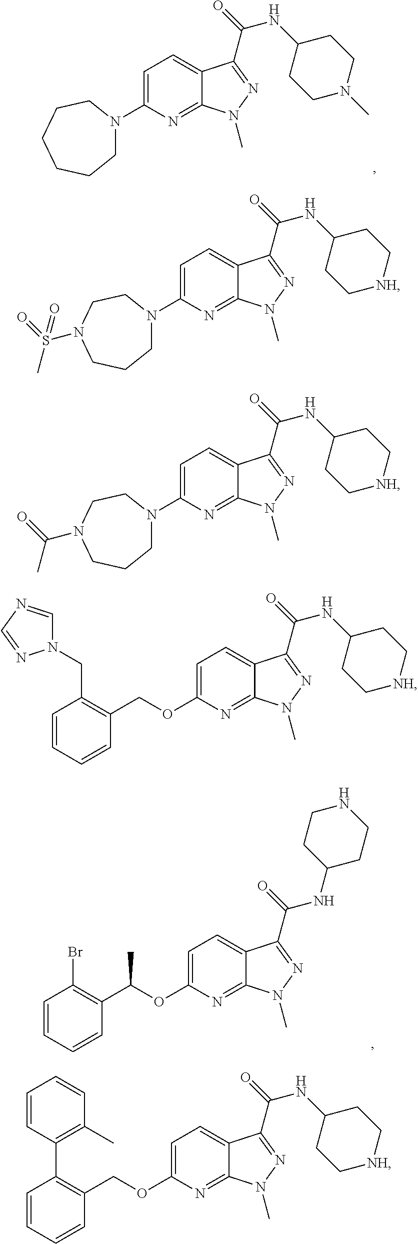

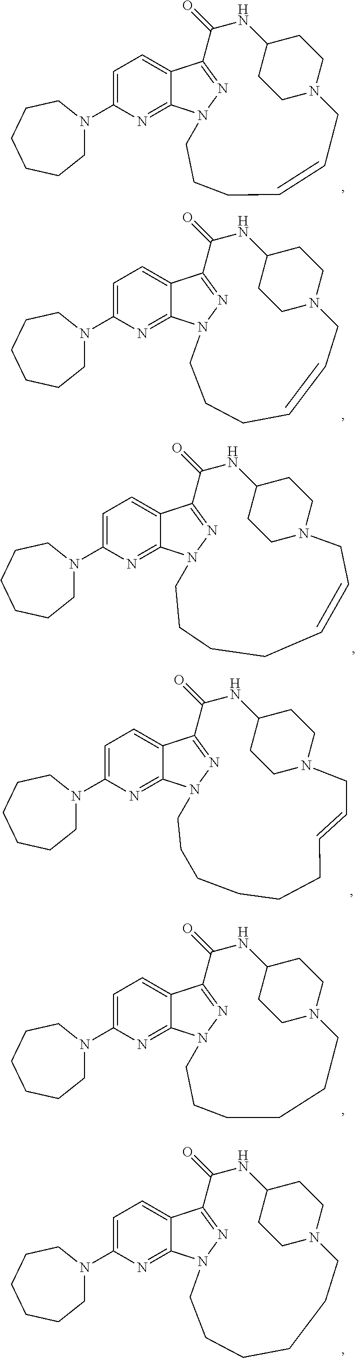

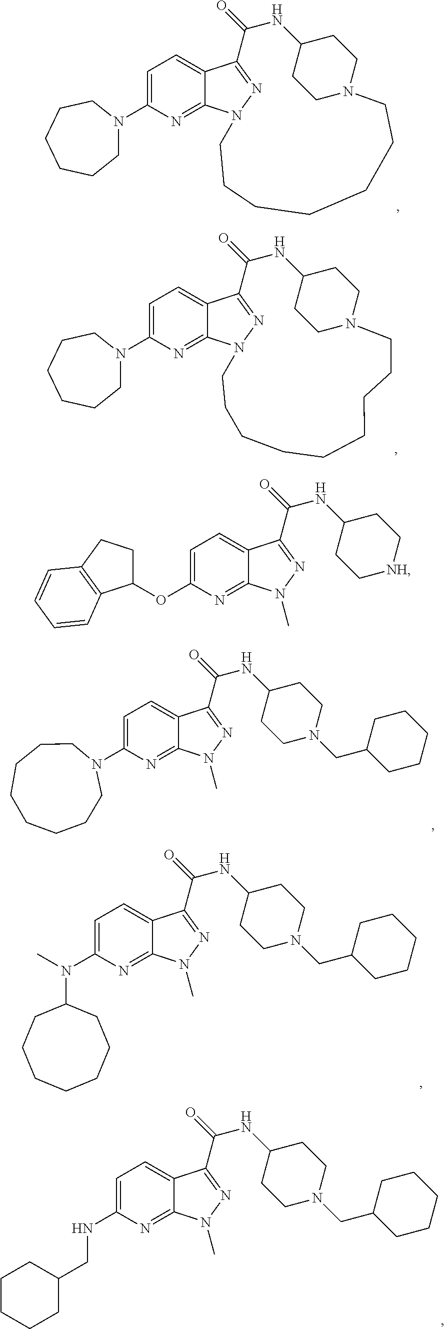

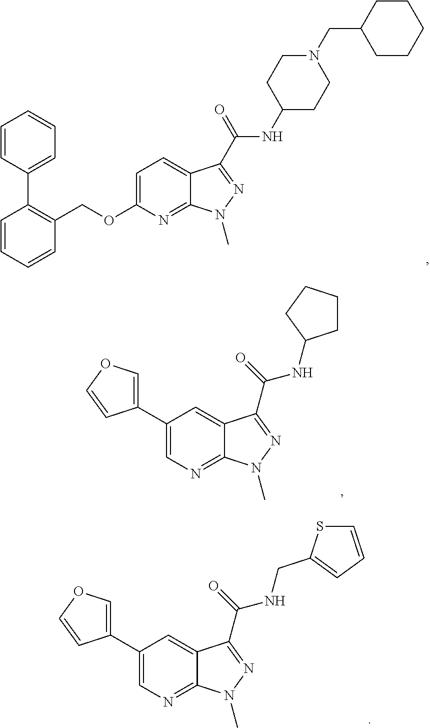

The compounds of the invention can be prepared by the methods schematically described below and by illustrative synthetic methods provided in the Examples below, with appropriate substitution of starting material, as necessary for a particular compound of interest.

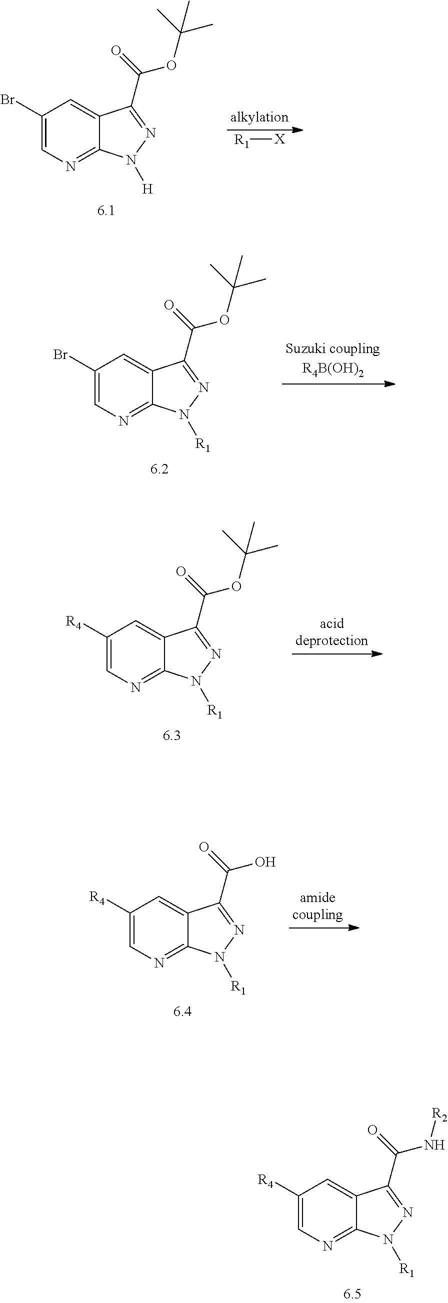

To a solution of 6.1 is added alkylhalide R₁X to provide 6.2. Suzuki coupling conditions is used to couple aryl or alkyl boronic acids to provide 6.3. Standard acid deprotection of t-butyl ester followed by amide coupling provides 6.5.

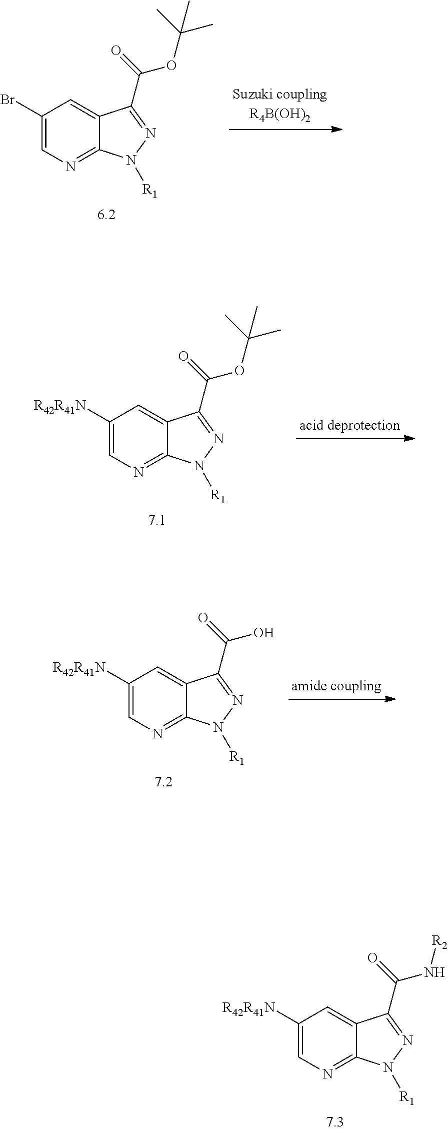

Buchwald coupling conditions is used to couple 6.2 to aryl or alkyl amines to provide 7.1. Standard acid deprotection of t-butyl ester followed by amide coupling provides 7.3. R₄₁ and R₄₂ are amine substituents disclosed herein, e.g., in Formula I.

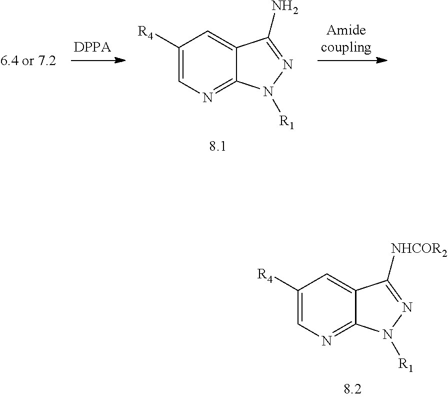

Treatment of 6.4 or 7.2 with diphenlphosphorylazide (DPPA) provides 8.1. Amide coupling provides 8.2.

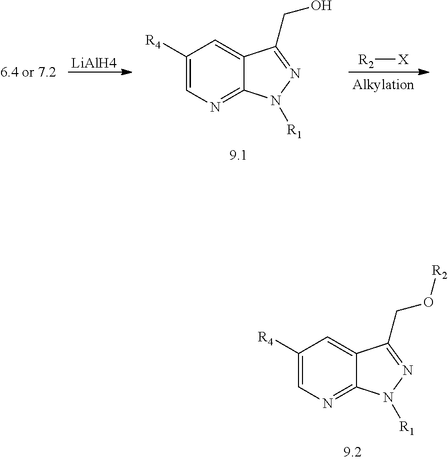

Treatment of 6.4 or 7.2 with LiAlH₄ provides 9.1. Deprotonation followed by alkylation provides 9.2.

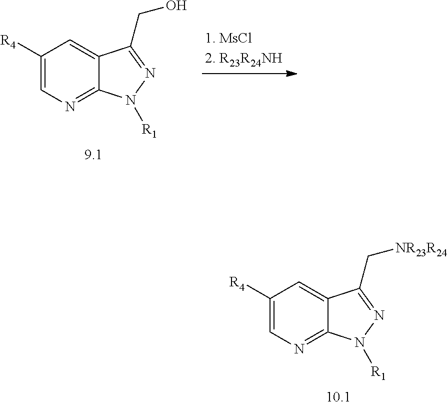

Treatment of 9.1 with MsCl followed by displacement with HNR₂₃R₂₄ (where N, R₂₃, and R₂₄ together form a group defined by R₂ as defined hereinabove, e.g., in Formulas I, IA and IB) provides 10.1. An alcohol such as R₂—OH, R₂CH₂OH, or the like can similarly be used to make compounds of, e.g., e.g., in Formulas I, IA and IB.

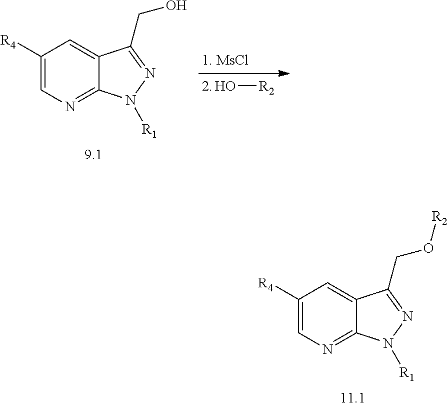

Treatment of 9.1 with MsCl followed by displacement with HO—R₂ provides 11.1.

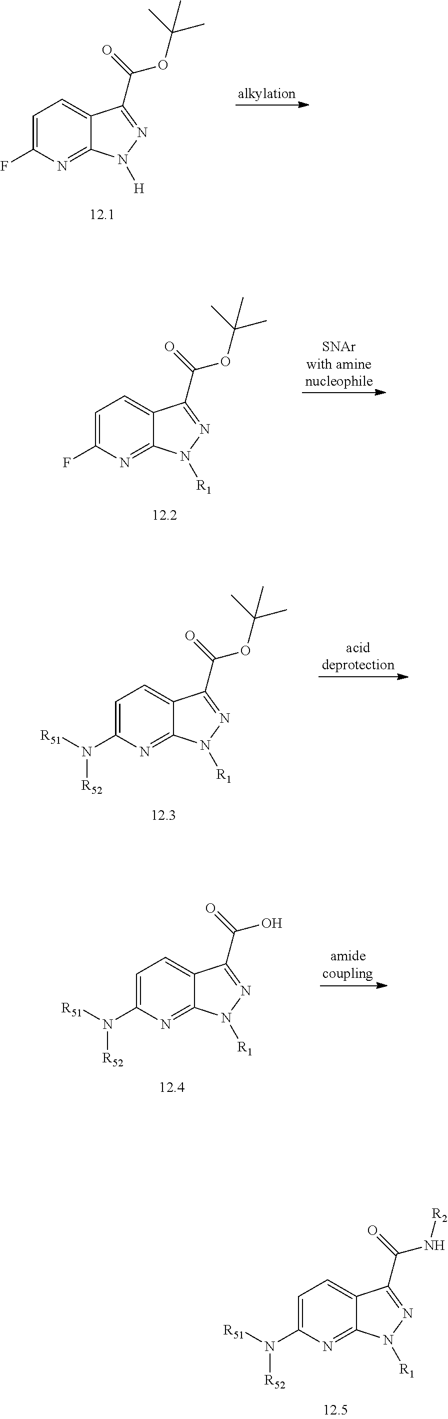

To a solution of 12.1 is added alkylhalide R₁X to provide 12.2. “SNAr” conditions are used to displace fluoride with amines to provide 12.3. Standard acid deprotection of t-butyl ester followed by amide coupling provides 12.5.

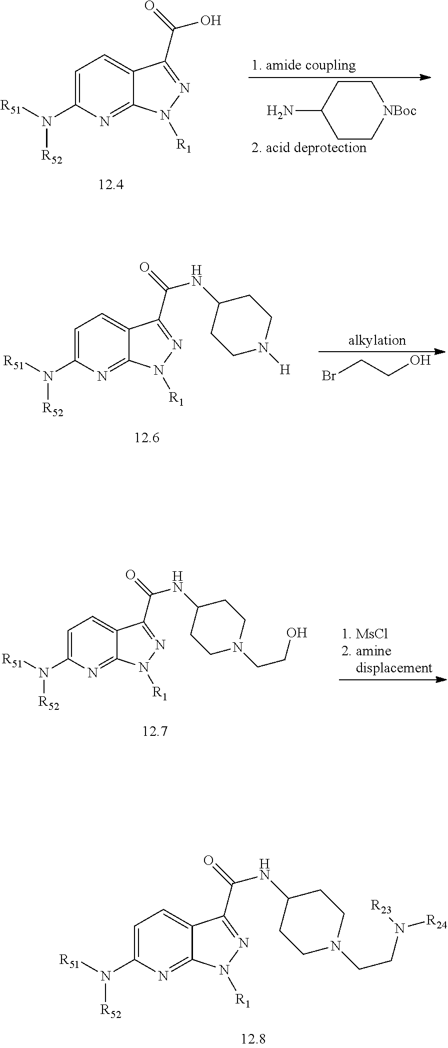

Amide coupling to 12.4 with tert-butyl 4-aminopiperidine-1-carboxylate followed by standard acid deprotection of t-butyl ester provides 12.6. Alkylation with 2-bromo ethanol

provides 12.7. Mesylation of 12.7 followed by amine displacement provides 12.8.

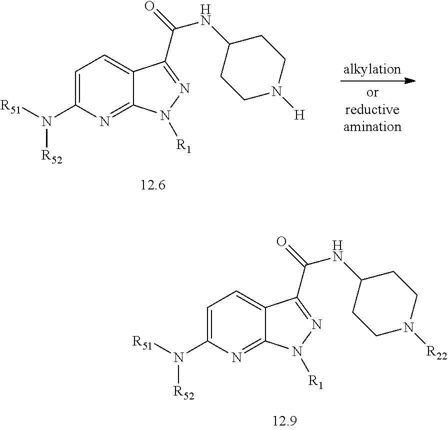

Alternatively, 12.6 can be alkylated via standard alkylation or reductive amination to provide 12.9, when R₂₂ is substituted or unsubstituted alkyl.

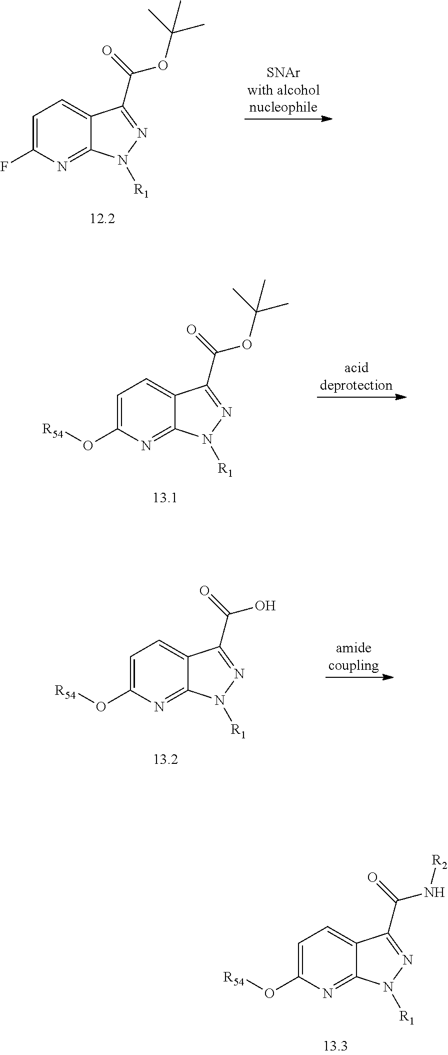

To a solution of 12.2 is added alcohol to displace fluoride to provide 13.1. Standard acid deprotection of t-butyl ester followed by amide coupling provides 13.3.

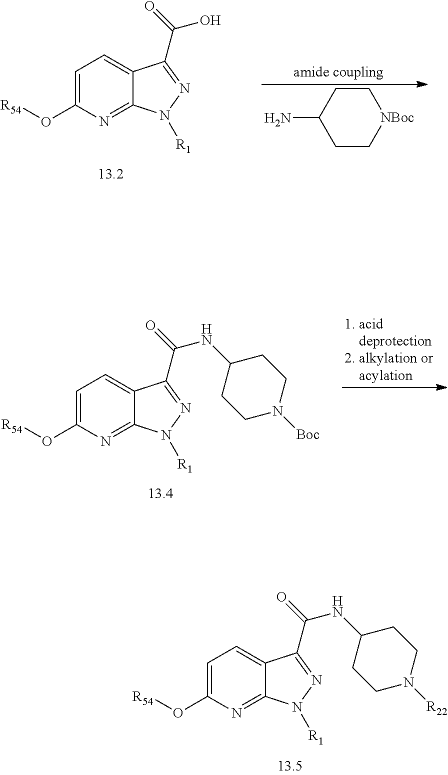

Amide coupling to 13.2 with tert-butyl 4-aminopiperidine-1-carboxylate provides 13.4. Standard acid deprotection of t-butyl ester followed by alkylation or acylation provides 13.5.

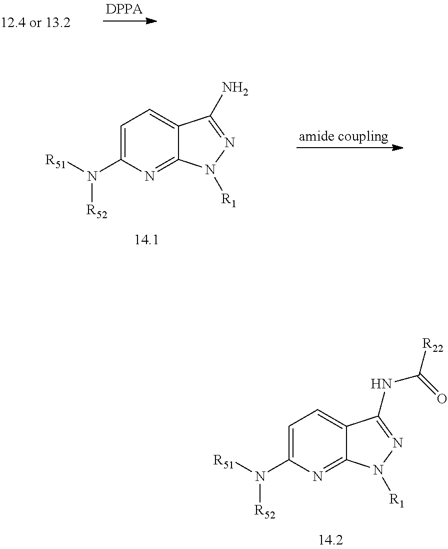

Treatment of 12.4 or 13.2 with DPPA provides 14.1. Amide coupling provides 14.2.

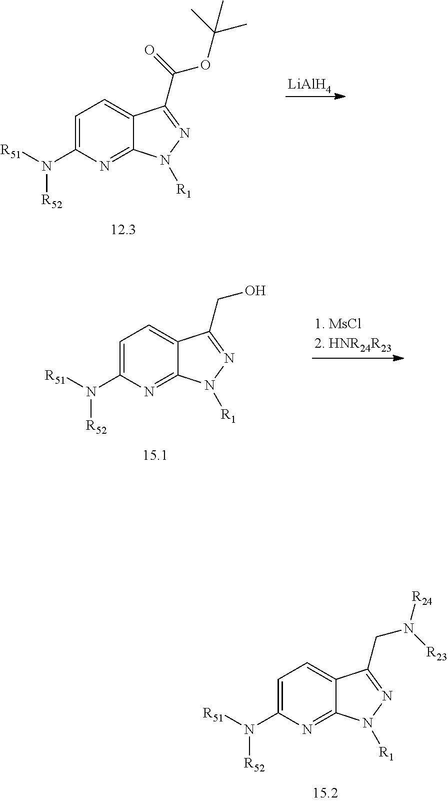

Treatment of 12.3 with LiAlH₄ provides 15.1. Mesylation followed by displacement with HNR₂₄R₂₃ (where N, R₂₄, and R₂₃ together form R₂ as defined hereinabove, e.g., in Formula IA or IC) provides 15.2.

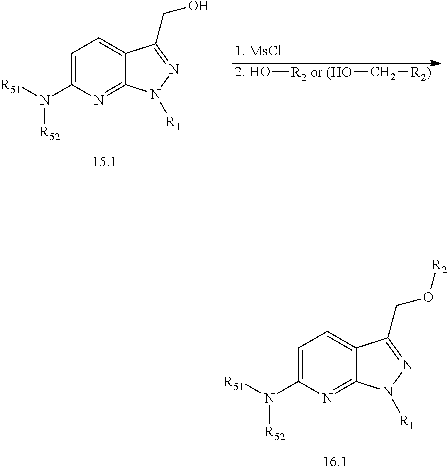

Treatment of 15.1 with MsCl followed by displacement with HO—R₂ or HO—CH₂—R₂ provides 16.1 or its homolog.

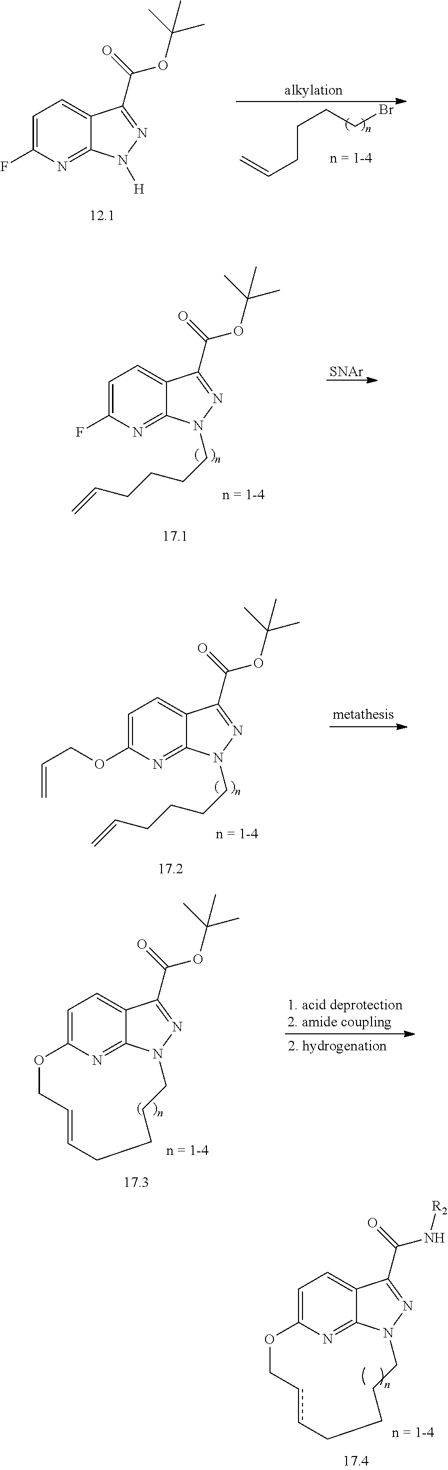

To a solution of 12.1 is added alkylhalide R₁X to provide 17.1. SNAr conditions are used to displace fluoride with benzylalcohol to provide 17.2. Olefin metathesis is used to provide cyclized product 17.3. Standard acid deprotection of t-butyl ester followed by amide coupling provides and hydrogenation provides 17.4.

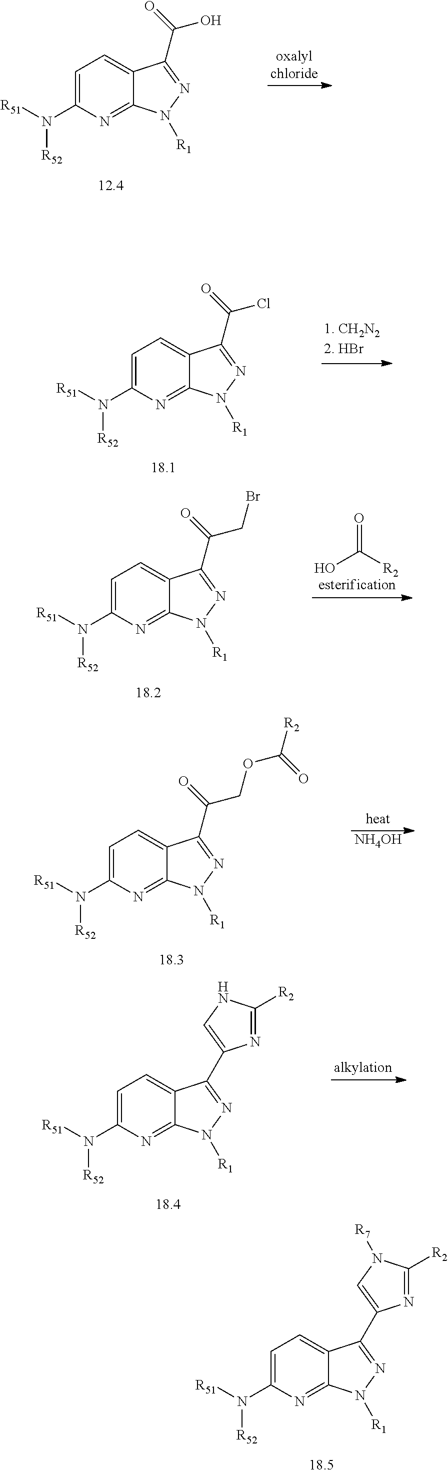

To a solution of 12.4 is added oxalyl chloride to provide 18.1. Treatment with diazomethane followed by HBr provides 18.2. Esterification of R₂COOH followed by heating provides imidazole 18.4. Alkylation provides 18.5.

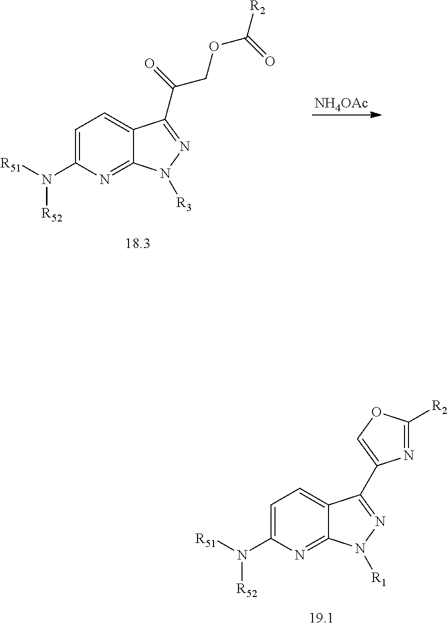

Treatment of 18.3 with ammonioum acetate provides 19.1.

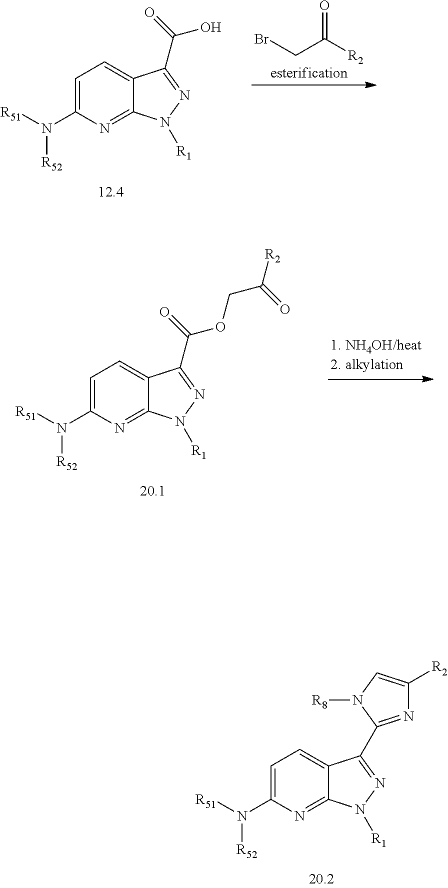

Treatment of 12.4 bromomethylketone provides 20.1. Heating followed by alkylation provides 20.2.

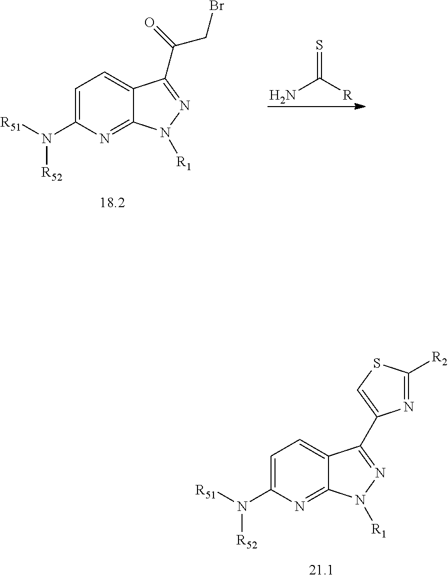

Treatment of 18.2 with thioamides provides 21.1.

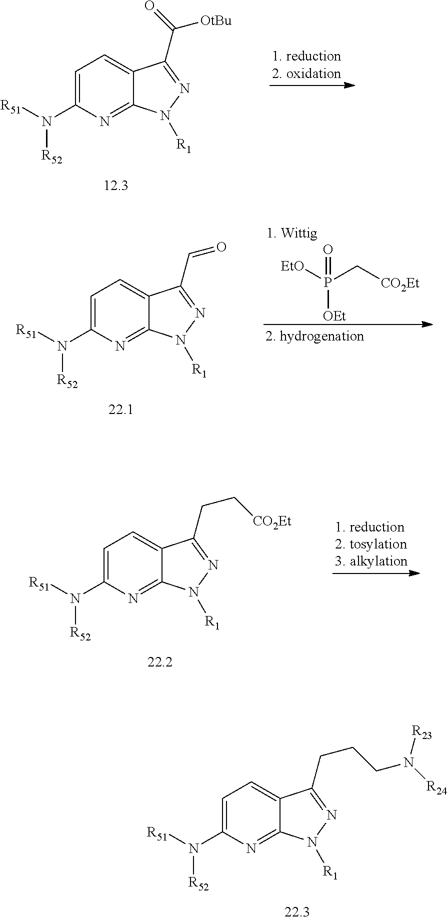

Treatment of 12.3 with LiAlH₄ followed by oxidation conditions provides aldehyde 22.1. Treatment with ethyl 2-(diethoxyphosphoryl)acetate under Wittig conditions followed by hydrogenation provides 22.2. Reduction of the ester to the alcohol, followed by tosylation and displacement with amines provides 22.3.

### III. Flaviviridae Virus Infections Amenable to Treatment

In certain aspects, the present invention provides methods for treating diseases relating to Flaviviridae virus infections. One exemplary method of treating a host infected with a virus from the Flaviviridae family of viruses provided by the invention, among others, includes: administering to the host a therapeutically effective amount of a compound of the invention to inhibit HCV or reduce the viral load in the host. In one embodiment, the compound of the present invention administered is selected from compounds of Formulas I, IA-D, II, IIA-J, III, IV, IVA-B, V, VA, VI, VIA-B, VII, VIIA, VIII, VIIIA, IX, and IXA-J and pharmaceutically acceptable salts or prodrugs thereof. In one embodiment, the compounds of the present invention are isolated EBP841, EBP1310, EBP1047, EBP1489, EBP1452, EBP1172, or EBP1456 and pharmaceutically acceptable salts or prodrugs thereof. In various embodiments, the compounds of the present invention are administered as their pharmaceutical compositions.

Compounds of the invention are useful in the treatment of viral infections, where the virus is a Flaviviridae family virus, which family includes, but is not limited to, flaviviruses, pestiviruses and hepatitis C viruses. Other Flaviviridae viruses include yellow fever virus (YFV); Dengue virus, including Dengue types 1-4; Japanese Encephalitis virus; Murray Valley Encephalitis virus; St. Louis Encephalitis virus; West Nile virus; tick-borne encephalitis virus; Hepatitis C virus (HCV); Kunjin virus; Central European encephalitis virus; Russian spring-summer encephalitis virus; Powassan virus; Kyasanur Forest disease virus; and Omsk hemorrhagic fever virus. Thus, where the specification below refers to HCV, such a reference is only for clarity and is not intended to limit the disclosure to HCV, because the methods and compositions of the invention can be applied to any Flavivirdae virus

Embodiments of the present invention include methods of treating an infection by a virus of the Flaviviridae family of viruses. In particular, a compound of the invention described herein can be used to treat an infection by a virus of the Flaviviridae family of viruses. In an embodiment, the present disclosure provides a method of treating a host infected with a virus from the Flaviviridae family of viruses by administering to the host a therapeutically effective amount of a compound of the invention in one or more doses to reduce the viral load in the host. Embodiments of the present invention also include methods of prophylactically treating an infection by a virus of the Flaviviridae family of viruses. In particular, a compound of the invention can be used as described herein to prophylactically treat an infection by a virus of the Flaviviridae family of viruses.

In an embodiment, a compound of the invention as described herein is used in combination with another agent (e.g. an anti-viral agent) to treat an infection with a virus from the Flaviviridae family of viruses. In an embodiment, a compound of the invention described herein is used in combination with another agent (e.g. an anti-viral agent) to treat an infection with a virus from the Flaviviridae family of viruses prophylactically.

In an embodiment, an effective amount of a compound of the invention is an amount that, when administered in one or more doses to a host (e.g., human) in need thereof, reduces HCV or other Flaviviridae virus viral load in the individual by at least about 10%, at least about 50%, at least about 75%, at least about 80%, or at least about 90%, or more, compared to the viral load in the individual not treated with a compound of the invention. Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT) sold by Chiron Corporation under the trade name Procleix®, which NAT simultaneously tests for the presence of HIV-1 and HCV. See, e.g., Vargo et al. (2002) Transfusion 42:876-885.

Various Flaviviridae viruses, including but not limited to HCV, can severely damage the liver of infected patients. Accordingly, the present invention provides methods for preventing liver damage and, in some patients, restoring liver function. Thus, In some embodiments, an effective amount of a compound of the invention is an amount that, when administered in one or more doses to a host (e.g., human) in need thereof, increases liver function in the individual by at least about 10%, at least about 25%, at least about 50%, at least about 75%, at least about 90%, or more, compared to the liver function in the individual not treated with a compound of the invention. In some embodiments, an effective amount of a compound of the invention is an amount that, when administered in one or more doses to a host (e.g., a human) in need thereof, reduces liver fibrosis in the host by at least about 10%, at least about 25%, at least about 50%, at least about 75%, at least about 90%, or more, compared to the degree of liver fibrosis in the individual not treated with a compound of the invention.

Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the transient elastography, METAVIR, Knodell, Scheuer, Ludwig, and Ishak scoring systems.

The transient elastography fibrosis scoring system is suitable for use in determining whether a patient is in need of treatment or is responding to treatment in accordance with the methods of the invention and was developed by Thierry Poynard and marketed primarily in the EU but also in the US. It is often used when an invasive liver biopsy is risky. The marketed product for this scoring system is called FibroScan, and the system provides a measure of liver stiffness.

The METAVIR scoring system is based on an analysis of various features of a liver biopsy, including fibrosis (portal fibrosis, centrilobular fibrosis, and cirrhosis); necrosis (piecemeal and lobular necrosis, acidophilic refraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. Thus, the scoring is such that higher the score, the more severe the liver tissue damage. See Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. See Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of a therapy provided by the invention can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

a. HCV

In an embodiment, a compound of the invention for use in inhibiting HCV replication and treating HCV infection, is of particular interest. The HCV treatable in accordance with the methods of the invention may be of any genotype (genotype 1, 2, 3, 4, 5, 6, and the like), as well as subtypes of an HCV genotype (e.g., 1a, 1b, 2a, 2b, 3a, etc.). Because HCV genotype 1 is typically the most difficult to treat, the methods and compositions of the invention for treating infections by HCV genotype 1 and genotype 1 subtypes are of particular interest. However, methods for treating other HCV genotypes are still needed, and such methods are provided by the invention. Thus, in an embodiment, the present invention provides methods of treating a flavivirus infection, e.g., an HCV infection, and methods of reducing liver fibrosis that may occur as sequelae of an HCV infection.

Embodiments of the present disclosure provide methods, compounds, and pharmaceutical formulations useful in the treatment of patients suffering from a viral infection. In one embodiment, the patient is infected with HCV but is not known to be infected with another virus, including, but not limited to, HIV. In another embodiment, the patient is infected with HCV and one or more additional viruses, including, but not limited to, HIV. In one embodiment, the patient is treated for a viral infection by administering only a single compound of the invention as described herein as useful in the treatment of HCV infection. In another embodiment, the patient is treated for a viral infection by administering both a compound of the invention described herein as useful in the treatment of HCV infection as well as one or more additional agents known to be useful in the treatment of viral infection.

(i) Genotype 1b

HCV Genotype 1b occurs in 15-20% of patients in the United States. Subtype 1b is difficult to eradicate using current medications. This type is most prevalent in Europe, Turkey, and Japan. The present invention provides methods for treating HCV Genotype 1b infection.

(ii) Other Genotypes

The most commonly used classification of Hepatitis C virus has HCV divided into the following genotypes (main types): 1, 2, 3, 4, 5, 6, 7, 8, 9, 10 and 11. HCV genotypes can be broken down into sub-types, some of which include: 1a, 1b, 1c; 2a, 2b, 2c; 3a, 3b; 4a, 4b, 4c, 4d, 4e; 5a; 6a; 7a, 7b; 8a, 8b; 9a; 10a; and 11a. Of these, 1a is mostly found in North and South America but also common in Australia. 1b, as noted above, is mostly found in Europe and Asia. 2a is the most common genotype 2 in Japan and China. 2b is the most common genotype 2 in the U.S. and Northern Europe. 2c is the most common genotype 2 in Western and Southern Europe. 3a is highly prevalent here in Australia (40% of cases) and South Asia. 4a is highly prevalent in Egypt. 4c is highly prevalent in Central Africa. 5a is highly prevalent only in South Africa. 6a is restricted to Hong Kong, Macau and Vietnam. 7a and 7b are common in Thailand. 8a, 8b and 9a are prevalent in Vietnam. 10a and 11a are found in Indonesia.

More particularly, genotype 1a occurs in 50-60% of patients in the United States. This type is difficult to eradicate using current medications. Genotype 1c occurs in less than 1% of patients in the United States. Genotypes 2a, 2b, and 2c occur in 10-15% of patients in the United States. These subtypes are widely distributed and are most responsive to medication. Genotypes 3a and 3b occur in 4-6% of patients in the United States. These subtypes are most prevalent in India, Pakistan, Australia, and Scotland. Genotype 4 occurs in less than 5% of patients in the United States. It is most prevalent in the Middle East and Africa. Genotype 5 occurs in less than 5% of patients in the United States. It is most prevalent in South Africa. Genotype 6 occurs in less than 5% of patients in the United States. It is most prevalent in Hong Kong and Macao.

The methods of the invention are also efficacious against these and other HCV genotypes and subtypes.

### IV. Pharmaceutical Compositions, Unit Dose Forms, and their Administration

In certain aspects, the present invention provides pharmaceutical compositions comprising, or in the alternative consisting essentially of, one or more compounds of the present invention and optionally one or more other anti-viral agents as identified herein and formulated with one or more pharmaceutically acceptable excipients, diluents, carriers and/or adjuvants. In one embodiment, the one or more compounds of the present invention are selected from compounds of Formulas I, IA-D, II, IIA-J, III, IV, IVA-B, V, VA, VI, VIA-B, VII, VIIA, VIII, and VIIIA, IX, and IXA-J and pharmaceutically acceptable salts or prodrugs thereof. In one embodiment, the one or more compounds of the present invention are selected from isolated EBP841, EBP1310, EBP1047, EBP1489, EBP1452, EBP1172, or EBP1456, and pharmaceutically acceptable salts or prodrugs thereof. In addition, embodiments of the pharmaceutical compositions of the present invention include such compounds of the invention formulated with one or more pharmaceutically acceptable auxiliary substances. In particular, one or more compounds of the invention can be formulated with one or more pharmaceutically acceptable excipients, diluents, carriers, and/or adjuvants to provide an embodiment of a pharmaceutical composition of the invention.

In an embodiment, a compound of the invention is combined with another anti-viral agent to prepare a pharmaceutical composition of the invention, and the pharmaceutical composition can include one or more pharmaceutically acceptable excipients, diluents, carriers and/or adjuvants.

In an embodiment, a compound of the invention (which may also be referred to below as a “drug”) can be formulated with one or more pharmaceutically acceptable excipients, diluents, carriers, and/or adjuvants to provide a formulation useful in the methods of the invention.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7, Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present invention, a compound of the invention is formulated into pharmaceutical compositions by combination with appropriate, pharmaceutically acceptable carriers or diluents, and is formulated into preparations in solid, semi-solid, liquid or gaseous forms, such as tablets, capsules, powders, granules, ointments, solutions, suppositories, injections, inhalants and aerosols.

In pharmaceutical dosage forms, a compound of the invention may be administered in the form of its pharmaceutically acceptable salts, or a compound of the invention may be used alone or in appropriate association, as well as in combination, with other pharmaceutically active compounds. The following pharmaceutical formulations, unit dose forms, methods for their preparation, and excipients are merely exemplary and are in no way limiting.

For oral preparations, a compound of the invention can be used alone or in pharmaceutical formulations of the invention comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Pharmaceutical formulations and unit dose forms suitable for oral administration are particularly useful in the treatment of chronic conditions, viral infections, and therapies in which the patient self-administers the drug. For acute infections and life-threatening conditions, particularly those requiring hospitalization, intravenous formulations are desirable, and the present invention provides such formulations as well.

The invention provides pharmaceutical formulations in which the compound can be formulated into preparations for injection in accordance with the invention by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Aerosol formulations provided by the invention can be administered via inhalation. For example, embodiments of the pharmaceutical formulations of the invention comprise a compound of the invention formulated into pressurized acceptable propellants such as dichlorodifluoromethane, propane, nitrogen and the like.

Suppositories of the invention can be prepared by mixing a compound of the invention with any of a variety of bases such as emulsifying bases or water-soluble bases. Embodiments of this pharmaceutical formulation of a compound of the invention can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds of the invention. Similarly, unit dosage forms for injection or intravenous administration may comprise a compound of the invention in a composition as a solution in sterile water, normal saline or another pharmaceutically acceptable carrier.

Embodiments of the pharmaceutical formulations of the invention include those in which a compound of the invention is formulated in an injectable composition. Injectable pharmaceutical formulations of the invention are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the invention.

In an embodiment, a compound of the invention is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound of the invention can be accomplished using any of a variety of refillable, pump systems. Pumps provide consistent, controlled release over time. In some embodiments, a compound of the invention is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, and the like.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a compound of the invention is delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for a compound of the invention are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present invention include those that comprise a sustained-release or controlled release matrix. In addition, embodiments of the present invention can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) are delivered in a controlled release system. For example, a compound of the invention may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present invention (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

Thus, the invention provides a variety of pharmaceutical formulations, unit dose forms, and drug delivery devices for administering a compound of the invention in accordance with the methods of the invention. These include, but are not limited to, tablets, capsules, and suspensions suitable for oral administration; formulations suitable for intramuscular and/or intravenous administration; lotions, creams, suspensions, gels, and treated patches and/or bandages suitable for topical application; and pumps and implantable depot formulations and devices for continuous administration of a compound of the invention.

As is clear from the previous section, the present invention provides methods and compositions for the administration of a compound of the invention to a host (e.g., a human) for the treatment of a viral infection. In various embodiments, these methods of the invention span almost any available method and route suitable for drug delivery, including in vivo and ex vivo methods, as well as systemic and localized routes of administration.

Thus, routes of administration applicable to the methods of the invention include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In general, routes of administration suitable for the methods of the invention include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The compounds of the invention can also be delivered to the subject by enteral administration. Enteral routes of administration include, but are not limited to, oral and rectal (e.g., using a suppository) delivery.

Methods of administration of the inhibiting agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods of the invention, a compound of the invention will be administered orally on a continuous, daily basis, at least once per day (QD), and in various embodiments two (BID), three (TID), or even four times a day, although compounds administered TID or more frequently can be more conveniently administered using the sustained release pharmaceutical formulations or other continuous delivery methods of the invention. Such daily administration will typically be continued for at least a week, often for at least four weeks, sometimes for at least 3 months, and in some cases for a year or longer. Typically, the therapeutically effective daily dose will be at least 1 mg to no more than 5 g; for example, daily doses of 10 mg, 100 mg, 250 mg, 500 mg, 1 g, or 2.5 g may be administered, depending on the particular compound and the method of administration selected. Unit doses suitable for oral administration will typically be in the form of a tablet or capsule containing 100 mg, 250 mg, or 500 mg of a compound of the invention. Illustrative compounds of the invention suitable for use in such unit dose forms include, without limitation, compounds with EBP numbers, 699, 700, 701, 749, 824, 827, 832, 833, 835, 836, 838, 839, 841, 910, 963, 1040, 1046, 1047, 1075, 1203, 1222, 1225, 1234, 1235, 1236, 1296, 1300, 1305, 1306, 1307, 1310, 1424, 1425, 1426, 1468, 1469, 1471, 1473, 1474, 1475, 1478, 1479, 1486, 1487, 1488, 1489, 1556, 1557, 1558, 1559, 1560, 1561, 1562, 1581, 1594, 1595, 1596, 1597, 1598, 1604, 1609, 1619, 1620, 1621, 1622, and 1632-1659. Of these, compounds 910, 963, 1040, 1047, 1075, 1203, 1222, 1225, 1234, 1235, 1236, 1296, 1300, 1305, 1306, 1307, 1310, 1424, 1425, 1426, 1468, 1469, 1471, 1473, 1474, 1475, 1478, 1479, 1486, 1487, 1488, 1489, 1556, 1557, 1558, 1559, 1561, 1562, 1581, 1594, 1595, 1596, 1597, 1598, 1604, 1609, 1619, 1620, 1621, 1622, and 1632-1659 are especially noteworthy, as the data herein demonstrate that the compounds should be a safe and effective for treating HCV when administered orally QD, BID, or TID.

Dosing can be accomplished in accordance with the methods of the invention using capsules, tablets, oral suspension, suspension for subcutaneous or intra-muscular injection, suspension for intravenous infusion, gel or cream for topical application, or suspension intra-articular injection.

In one embodiment of the invention, a compound of the invention is administered to a patient in need of therapy to treat HCV infection. Various combination therapies of the invention for the treatment of HCV infection are described in Section V, below. In these combination therapies, a compound of the invention will be administered as described herein, and the other compound is administered in accordance with the administration schedule approved by the regulatory authorities.

In a further aspect, this invention provides the use of any one or more of the inventive compound or compositions of this invention for the preparation of a medicament for inhibiting or treating an HCV infection.

### V. Combination Therapies

The pharmaceutical formulations and unit dose forms described herein can be used in combination with other drugs, including other anti-viral drugs. Thus the methods of the invention include methods for treating a virus-induced (or other pathogen-induced) disease by administering two or more drugs, at least one of which is a compound of the invention and at least one of which is selected from the group consisting of (1) nucleoside analogs, including but not limited to ribavirin; (2) interferons; (3) thiazolides, including but not limited to nitazoxanide; (4) protease inhibitors; (5) polymerase inhibitors (both nucleoside and non-nucleoside inhibitors); (6) helicase inhibitors; (7) class C CpG toll-like receptor 7 and/or 9 antagonists; (8) amphipathic helix disruptors; (9) statins; (10) immunomodulators (including steroidal and non-steroidal immunomodulators); (11) anti-inflammatories; (12) an inhibitor of prenylation, including prenyltransferase inhibitors, including but not limited to another FTI, GGTI, or dual-acting FTI/GGTI; and/or (13) other agents, including agents for the treatment of side effects and/or pain relief. Each of these classes of other drugs that can be used in the combination therapies of the invention are discussed below.

### 1. Nucleoside Analogs

Nucleoside analogs that are suitable for use in a combination therapy of this invention include, but are not limited to, ribavirin, levovirin, taribavirin, isatoribine, an L-ribofuranosyl nucleoside as disclosed in U.S. Pat. No. 5,559,101 and encompassed by Formula I of that patent (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguanine, 2-amino-α-L-ribofuran[1′,2′:4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,2′:4,5]oxazoline, O²,O²-anhydro-β-L-arabinofuranosyluracil, 2′-deoxy-β-L-uridine, 3′5′-Di-O-benzoyl-2′deoxy-4-thio β-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of that patent; a compound as disclosed in U.S. Patent Publication No. 2002/0058635, and encompassed by Formula I of that publication; a nucleoside analog as disclosed in WO 01/90121 A2 (Idenix); a nucleoside analog as disclosed in WO 02/069903 A2 (Biocryst Pharmaceuticals Inc.); and a nucleoside analog as disclosed in WO 02/057287 A2 or WO 02/057425 A2 (both Merck/Isis). Certain nucleoside analogs are DNA polymerase inhibitors, which are also discussed as a class below.

In one embodiment, the nucleoside analog used in a combination therapy of the invention is ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Other nucleoside analogs useful in the combination therapies of the invention include derivatives of ribavirin (see, e.g., U.S. Pat. No. 6,277,830). In one embodiment, the nucleoside analog used in a combination therapy of the invention is levovirin. Levovirin is the L-enantiomer of ribavirin, and exhibits the property of enhancing a Th1 immune response over a Th2 immune response. Levovirin is manufactured by ICN Pharmaceuticals. In one embodiment, the nucleoside analog used in a combination therapy of the invention is taribavirin. Taribavirin is a 3-carboxamidine derivative of ribavirin, and acts as a prodrug of ribavirin. It is efficiently converted to ribavirin by adenosine deaminases.

### 2. Interferons

Current medical practice to treat HCV infection typically employs either interferon-alpha monotherapy or combination therapy with ribavirin (such as Rebetol or Copegus) and either an interferon-alpha (such as interferon alpha 2b) or pegylated interferon (such as Pegasys, marketed by Roche, or PEG-Intron, marketed by Schering Plough). In accordance with the methods of the present invention, a compound of the invention is used in combination with one of these standard therapies to treat HCV infection.

Thus, the present invention provides combination therapies in which an interferon, e.g., interferon-alpha (IFN-α) is used in combination with a compound of the invention. Any known IFN-α can be used in the treatment methods of the invention. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α. Thus, essentially any IFN-α that has antiviral properties, as described for naturally occurring IFN-α, can be used in the combination therapies of the invention.

Suitable alpha interferons for purposes of the invention include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceuticals, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3a mixture of natural alpha interferons made by Interferon Sciences and available from the Purdue Frederick Co., Norwalk, Conn., under the Alferon tradename.

The term “IFN-α” also encompasses consensus IFN-α. Consensus IFN-α (also referred to as “CIFN” and “IFN-con” and “consensus interferon”) encompasses, but is not limited to, the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, Three Rivers Pharmaceuticals, Warrendale, Pa.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. In an embodiment, the at least one additional therapeutic agent is CIFN.

In various embodiments of the combination therapies of the invention, fusion polypeptides comprising an IFN-α and a heterologous polypeptide are used. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present disclosure are gene-shuffled forms of IFN-α. See, e.g., Masci et al. (2003) Curr. Oncol. Rep. 5:108-113. Other suitable interferons include Multiferon (Viragen), Medusa Interferon (Flamel Technology), Locteron (Octopus), and Omega Interferon (Intarcia/Boehringer Ingelheim).

The term “IFN-α” also encompasses derivatives of IFN-α that are derivatized (e.g., are chemically modified relative to the naturally occurring peptide) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Thus, in some embodiments of the combination therapies of the invention, the IFN-α has been modified with one or more polyethylene glycol moieties, i.e., PEGylated. The PEG molecule of a PEGylated IFN-α polypeptide is conjugated to one or more amino acid side chains of the IFN-α polypeptide. In an embodiment, the PEGylated IFN-α contains a PEG moiety on only one amino acid. In another embodiment, the PEGylated IFN-α contains a PEG moiety on two or more amino acids, e.g., the IFN-α contains a PEG moiety attached to two, three, four, five, six, seven, eight, nine, or ten different amino acid residues. IFN-α may be coupled directly to PEG (i.e., without a linking group) through an amino group, a sulfhydryl group, a hydroxyl group, or a carboxyl group. In various embodiments of the invention, an interferon, ribavirin, and a compound of the invention are administered in combination to treat HCV infection.

### 3. Thiazolides

A number of thiazolide derivatives are in development for the treatment of Flaviviridae virus, including but not limited to HCV, infection, and in accordance with the methods of the present invention, co-administration of a compound of the invention and a thiazolide, including, but not limited to, nitazoxanide (Alinia, Romark Laboratories, or other sustained release formulations of nitazoxanide or other thiazolides) is efficacious in the treatment of HCV. Nitazoxanide administration in accordance with the combination therapies of the invention can be, for illustration and without limitation, 500 mg po BID. In one embodiment, an interferon alpha and/or a nucleoside analog such as ribavirin is/are also employed in this combination therapy. Other doses, other thiazolides, or other formulations of nitazoxanide or another thiazolide, such as sustained release formulations, can also be used in the combination therapies of the invention.

### 4. Protease Inhibitors

A number of HCV protease inhibitors are in development for the treatment of HCV infection, and in accordance with the methods of the present invention, co-administration of a compound of the invention and an HCV protease inhibitor is efficacious in the treatment of HCV and other Flaviviridae virus infections. In one embodiment, an interferon alpha and/or a nucleoside analog such as ribavirin is/are also employed in this combination therapy. Suitable HCV protease inhibitors include, but are not limited to, telaprevir (VX-950, Vertex), BILN 2061 and BI 12202 (Boehringer Ingelheim), boceprevir (SCH 503034, Schering Plough), ITMN191 (Roche/InterMune/Array BioPharma), MK-7009 (Merck), TMC435350 (Tibotec/Medivir), ACH-1095 and ACH-806 (Achillion/Gilead), and other inhibitors of NS3/NS4A protease, including, but not limited to, compounds in development by Presidio.

Thus, in one embodiment, an HCV NS3 inhibitor is administered in combination with a compound of the invention to treat HCV. Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301 A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH6 (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); and an NS3 protease inhibitor as disclosed in any one of U.S. Patent Publication Nos. 2003019067, 20030187018, and 20030186895.

In an embodiment, the NS3 inhibitor used in a combination therapy of the invention is a member of the class of specific NS3 inhibitors, e.g., NS3 inhibitors that inhibit NS3 serine protease activity and that do not show significant inhibitory activity against other serine proteases such as human leukocyte elastase, porcine pancreatic elastase, or bovine pancreatic chymotrypsin, or cysteine proteases such as human liver cathepsin B.

### 5. Polymerase Inhibitors

A number of HCV RNA polymerase (NS5B) inhibitors are in development for the treatment of HCV infection, and in accordance with the methods of the present disclosure, co-administration of a compound of the invention and an HCV RNA polymerase inhibitor is efficacious in the treatment of HCV. In one embodiment, an interferon alpha and/or a nucleoside analog such as ribavirin and/or an HCV protease inhibitor is/are also employed in this combination therapy. Suitable HCV RNA polymerase inhibitors include, but are not limited to, valopicitabine (NM283, Idenix/Novartis), HCV-796 (Wyeth/ViroPharma), R1626 (Roche), R7128 (Roche/Pharmasset), GS-9190 (Gilead), MK-0608 (Merck), PSI-6130 (Pharmasset), and PFE-868,554 (PFE).

Thus, in one embodiment, an NS5B inhibitor is administered in combination with a compound of the invention to treat HCV infection. Suitable HCV non-structural protein-5 (N55; RNA-dependent RNA polymerase) inhibitors include, but are not limited to, a compound as disclosed in U.S. Pat. No. 6,479,508; a compound as disclosed in any of PCT Patent Application Nos. PCT/CA02/01127, PCT/CA02/01128, and PCT/CA02/01129; a compound as disclosed in U.S. Pat. No. 6,440,985; a compound as disclosed in WO 01/47883, e.g., JTK-003; a dinucleotide analog as disclosed in Zhong et al. (2003) Antimicrob. Agents Chemother. 47:2674-2681; a benzothiadiazine compound as disclosed in Dhanak et al. (2002) J. Biol. Chem. 277(41):38322-7; an NS5B inhibitor as disclosed in WO 02/100846 A1 or WO 02/100851 A2; an NS5B inhibitor as disclosed in WO 01/85172 A1 or WO 02/098424 A1; an NS5B inhibitor as disclosed in WO 00/06529 or WO 02/06246 A1; an NS5B inhibitor as disclosed in WO 03/000254; an NS5B inhibitor as disclosed in EP 1 256,628 A2; JTK-002; and JTK-109.

In one embodiment, the NS5 inhibitor used in the combination therapies of the invention is a member of the class of specific NS5 inhibitors, e.g., NS5 inhibitors that inhibit NS5 RNA-dependent RNA polymerase and that lack significant inhibitory effects toward other RNA dependent RNA polymerases and toward DNA dependent RNA polymerases.

### 6. Helicase Inhibitors

A number of agents targeting HCV NS3 helicase are in development, and compounds that suppress the HSV helicase-primase enzyme complex (such as ASP2151) are known and can be used in combination with a compound of the invention to treat viral infections. Thus, for treatment of Flaviviridae, including but not limited to HCV, virus infections, combinations of a compound of the invention with a helicase inhibitor are administered in various embodiments of the invention.

7. Class C CpG Toll-Like Receptor 7 and/or 9 Antagonists

A number of toll-like receptor (TLR) agonists are in development for the treatment of HCV infection, and in accordance with the methods of the present disclosure, co-administration of a compound of the invention and a TLR agonist can be efficacious in the treatment of HCV. In one embodiment, an interferon alpha and/or a nucleoside analog such as ribavirin and/or an HCV protease inhibitor and/or an HCV RNA polymerase inhibitor is/are also employed in this combination therapy. Suitable TLR agonists include, but are not limited to, TLR7 agonists (i.e., ANA245 and ANA975 (Anadys/Novartis)) and TLR9 agonists (i.e., Actilon (Coley) and IMO-2125 (Idera)).

### 8. Amphipathic Helix Disruptors and NS4B Inhibitors

In various embodiments of the invention, a compound of the invention is used in combination with another amphipathic helix disruptor and/or NS4B inhibitor disclosed herein and in PCT publication WO 2002/089731, PCT publication WO 2005/032329, PCT publication WO 2009/039248 (including but not limited to clemizole), PCT publication No. WO 2010/039195, PCT publication No. WO 2010/107739, and PCT publication No. WO 2010/107742, each of which is incorporated herein by reference.

### 9. Statins and Other HMG CoA Reductase Inhibitors

HMG CoA reductase inhibitors, including but not limited to statins, exert an antiviral effect (see Delang et al., 2009, Hepatology 50(1): 6-16; and Amet et al., Microbes and Infection 10(5): 471-480, both of which are incorporated herein by reference). In one embodiment of the combination therapies of the invention, an HMG CoA reductase inhibitor is used in combination with a compound of the invention to treat HCV infection. In various embodiments, the HMG CoA reductase inhibitor is a statin, including but not limited to lovastatin, simvastatin, atorvastatin, fluvastatin, and pravastatin. See, e.g., U.S. Pat. No. 7,223,787, incorporated herein by reference.

### 10. Immunomodulators

Steroid based immunomodulating therapies, including but not limited to treatment with methyprednisolone, are useful in the combination therapies of the invention, as are non-steroid immunomodulating therapies.

Non-steroid immunomodulating therapies useful in the combination therapies of the invention include administration of drugs from the following classes: inhibitors of inosine monophosphate dehydrogenase (IMPDH) and pro-drugs of inhibitors of IMPDH (mycophenolate mofetil); di-hydro orotate dehydrogenase inhibitors (teriflunomide; fingolimod; leflunomide) or pro-drugs of di-hydro orotate dehydrogenase inhibitors; monoclonal antibodies that target receptors on B-lymphocytes and/or T-lymphocytes (rituximab); compounds which cause selective apoptosis in dividing and non-dividing lymphocytes including purine nucleoside analog prodrugs (leustatin); compounds which can modulate the immune response resulting in a conversion from a Th1 to a Th2 response (glatiramer acetate); and inhibitors of folate metabolism (methotrexate).

### 11. Anti-Inflammatories

Anti-inflammatory therapies useful in the combination therapies of the invention include steroid-based therapies (methylprednisolone); treatment with tumor necrosis factor (TNF) antagonists (etanercept); and treatment with pyrimidine synthesis inhibitors (leflunomide)

### 12. Prenylation Inhibitors

A prenylation inhibitor (an inhibitor of prenylation) designates any compound, agent or treatment that inhibits (e.g., reduces or abolishes) the prenylation of proteins, more specifically the prenylation of proteins required for viral replication. Such inhibitors include more specifically any compound (e.g., antagonist) that inhibits a prenylation enzyme, particularly a prenyltransferase enzyme, more particularly a CAAX-prenyltransferase. Specific and preferred examples of such enzymes include geranylgeranyl transferase(s) (“GGTase”) and farnesyl transferase(s) (“FTase”). In a preferred embodiment, the FTase inhibitors (“FTIs”) or GGTase inhibitors (“GGTIs”) have an IC50 for the FTase or GGTase, respectively, which is below 1 mM and, more preferably, below 100 nM. The inhibitors can inhibit either GGTase or FTase, or both (i.e., dual inhibitors). Alternatively, a combination comprising a GGTase inhibitor and a FTase inhibitor can be used. Most preferred GGTase or FTase inhibitors are selective inhibitors, i.e., they are essentially active on GGT or FT with no substantial specific activity on other enzymes (IC50>20 μM). Most preferred prenyltransferase inhibitors for use in the present invention are AZD3409 and lonafarnib.

Illustrative GGTIs include FTI-277 and GGTI-298. Illustrative FTIs include 3-hydroxy-3-methyl glutaryl coenzyme A reductase inhibitors and HMG-CoA inhibitors (including the statins, discussed above). Other FTIs useful in the combination therapies of the invention include those described in the following publications: WO 98/54966; U.S. Pat. No. 6,096,757; Shih et al., Cancer Chemother Pharmacol (2000) 46: 387-393; WO 01/45740; WO 01/56552; WO 01/62234; WO 01/64199; EP 534546; Reiss, 1990, Cell 62: 81-8; James, 1993, Science 260: 1937-1942; Lerner, 1995, J. Biol. Chem. 270: 26802; WO 95/25086; EP 696593; PCT/GB96/01810; PCT/GB99/00369; WO 95/10516; WO 97/23478; and U.S. Pat. Nos. 5,874,442; 6,232,338; 7,101,897; and 7,342,016.

More specifically, FTIs useful in the combination therapies of the inventions include, but are not limited to: A-87049, A-176120, A-197574, A-228839, A-228839.25, A-345665, A-345877, A-373857, A-409100; ABT-100, ABT-839; Arglabin; Arglabin-DMA HCl; Arteminolide C; Artemisolide; 2-Benzoyloxycinnamaldehyde (BCA); AZD-3409; BIM-46068; BMS-191563, BMS-193269, BMS-214662, BMS-225975, BMS-316810; BNG-1; CH-222422; CP-609754, CP-663427; Dimethylaminoarglabin HCl; DMNQ-533; ER-51784, ER-51785; FTI-276, FTI-277, FTI-2148, FTI-2153, FTI-2600; Isorhamnetin; Isorhamnetol; J-104126, J-104134, J-104871; L-778123, L-779575; LB-42908; 3′-Methoxyquercetin; Methylflucidone; NSC-702818 (Tipifarnib), NSC-712392; OSI-754; PD-161956, PD-169451; R-115777; RPR-115135, RPR-130401, RPR-201764; SCH-400, SCH-207758, SCH-211618, SCH-226374, SCH-44342, SCH-54429, SCH-59228, SCH-66336 (lonafarnib; Sarasar), SCH-69955, SCH-69956, SCH-704742; TAN-1813; and XR-3054.

Other prenyltransferase inhibitors useful in the methods of the invention include 6-[Amino(4-chlorophenyl)-1-methyl-1H-imidazol-5-ylmethyl]-4-(3-chlorophenyl)-1-methyl-2(1H)-quinolinone (also identified as R115777, Tipifarnib or Zamestra™, whose FTase IC50 is 0.86 nM); 4-(3-chlorophenyl)-6-[(4-chlorophenyl)hydroxy(1-methyl-1H-imidazol-5-yl)methyl]-1-methyl-2(1H)-quinolinone; 6-[(4-chlorophenyl)hydroxy(1-methyl-1H-imidazol-5-yl)methyl]-4-(3-e-thoxyphenyl-1-methyl-2(1H)-quinolinone; 6-[(4-chlorophenyl)(1-methyl-1H-imidazol-5-yl)methyl]-4-(3-ethoxyph-enyl)-1-methyl-2(1H)-quinolinone monohydrochloride monohydrate; 6-[amino(4-chlorophenyl)(1-methyl-1H-imidazol-5-yl)methyl]-4-(3-ethoxyphenyl)-1-methyl-2(1H)-quinolinone; 6-[amino(4-chlorophenyl)(1-methyl-1H-imidazol-5-yl)methyl]-1-methyl-4-(3-propylphenyl)-2(1H)-quinolinone; (B)-6-[amino(4-chlorophenyl)(1-methyl-1H-imidazol-5-yl)methyl]-4-(3-chlorophenyl)-1-methyl-2(1H)-quinolinone (see patent applications WO9716443 and EP1162201.

### 13. Other Agents

In other embodiments of the methods of the present invention, co-administration of a compound of the invention and a compound from one of the following classes of compounds is used to treat HCV infection:
(a) cyclophilin inhibitors (i.e., NIM-811 (Novartis) and DEBIO-025 (Debiopharm));
(b) alpha-glucosidase inhibitor (i.e., Celgosivir (Migenix));
(c) agents targeting NS5A, including, but not limited to, A-831 (Arrow Therapeutics), AZD2836 (Astra Zeneca), and agents in development by XTL/Presidio or BMS (see PCT publications WO 2006/133326 and WO 2008/021928, incorporated herein by reference);
(d) agents targeting TBC1D20 and/or NS5A's interaction with TBC1D20 (see PCT publication WO 2007/018692 and U.S. patent application Ser. No. 11/844,993, incorporated herein by reference);
(e) agents targeting NS4B's GTPase activity (see PCT publication WO 2005/032329 and US patent application publication 2006/0199174, incorporated herein by reference);
(f) agents targeting PIP2 or BAAPP domains in HCV proteins, such as those found in NS4B and NS5A (see PCT publication No. WO 2010/148541);
(g) agents targeting HCV entry, assembly, or release, including antibodies to co-receptors;
(h) siRNAs, shRNAs, antisense RNAs, or other RNA-based molecules targeting sequences in HCV;
(i) agents targeting microRNA122 or other microRNAs modulating HCV replication;
(j) agents targeting PD-1, PD-L1, or PD-L2 interactions or pathway (see US patent application publications 20080118511, 20070065427, 20070122378, incorporated herein by reference);
(k) any agent approved for the treatment of HIV;
(l) any agent useful in the treatment of HBV (see Lok et al., April 2007, Gastroenterology 132: 1586-1594);
(m) side effect management agents, including but not limited to agents that are effective in pain management; agents that ameliorate gastrointestinal discomfort; analgesics, anti-inflammatories, antipsychotics, antineurotics, anxiolytics, hematopoietic agents, and any agent for palliative care of patients suffering from pain or any other side effect in the course of treatment with a subject therapy, including but not limited to palliative agents such as acetaminophen, ibuprofen, other NSAIDs, H2 blockers, proton pump inhibitors, and antacids.

The methods and compositions of the invention having now been described in detail, the following examples are provided to illustrate methods by which the compounds of the invention can be made and their anti-viral activity demonstrated. Activity against hepatitis and other viruses can be demonstrated in vitro through cell-based assays assessing the cytotoxicity and IC50 of a compound of the invention alone, and then in combination with other antiviral compounds. The cell lines used for these assays consist of cell lines conducive to growth of the respective viruses and may be laboratory-derived and/or patient-derived cell lines.

The examples herein are put forth so as to provide those of ordinary skill in the art with an illustrative disclosure and description of how to perform the methods and use the compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere. Any methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. Embodiments of the present disclosure will employ, unless otherwise indicated, techniques of synthetic organic chemistry, biochemistry, biology, molecular biology, recombinant DNA techniques, pharmacology, and the like, which are within the skill of the art. Such techniques are explained fully in the literature.

This disclosure is not limited to particular embodiments described, and the embodiment of the invention as such may, of course, vary. Before the embodiments of the present disclosure are described in detail, it is to be understood that, unless otherwise indicated, the present disclosure is not limited to particular materials, reagents, reaction materials, manufacturing processes, or the like, as such can vary. It is also possible in the present disclosure that steps can be executed in different sequence where this is logically possible.

### EXAMPLES

### 1. Synthesizing Compounds of the Present Invention

Compounds of the invention, including but not limited to the compounds shown in the tables above and compounds defined by the formulas herein, can be made using the methodology illustrated by the following methods.

A. Compounds of the present invention, compounds 1.6 and 1.8 (compounds described particularly by Formulas IA, IB, IC, II, and IIA-C), were synthesized as schematically shown and described below.

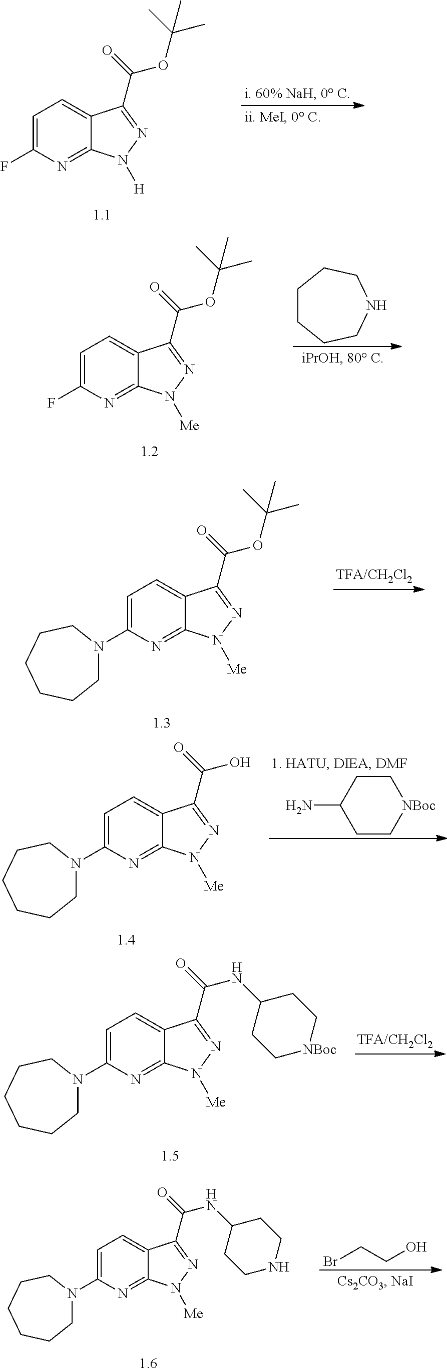

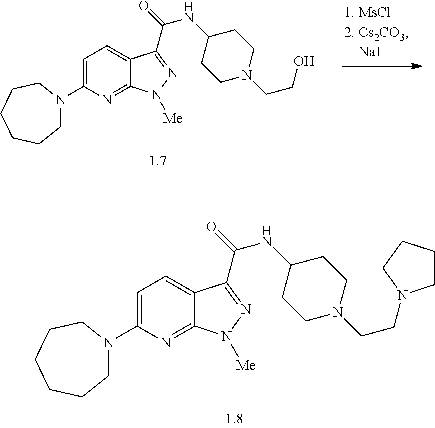

a. To a solution of 1.1 (0.5 g, 2.1 mmol) in THF (50 mL) at 0° C. was added 60% NaH (0.092 g, 2.3 mmol). After stirring at 0° C. for 30 min, MeI (0.143 mL, 2.3 mmol) was added dropwise. After stirring for 1 h, the solution was diluted with EtOAc (50 mL) and washed with saturated aqueous NaHCO₃ (3×20 mL). The organic solvents were removed in vacuo to provide crude 1.2 which was used without further purification.
b. To a solution of 1.2 (2.1 mmol) in iPrOH (50 mL) was added azepane (4.1 mmol). After heating the solution to 80° C. for 12 h, the solvent was removed in vacuo to provide crude 1.3. Purification via silica gel chromatography (10-20% EtOAc in hexanes) yielded two compounds with molecular weight corresponding to product, the higher Rf spot being the desired product 1.3.
c. After treatment of 1.3 (2.1 mmol) with TFA/CH₂Cl₂ (1:1) for 2 h, removal of solvent in vacuo provided 1.4 which was used without further purification.
d. To a solution of 1.4 (0.1 g, 0.5 mmol) in DMF (5 mL) was added tert-butyl 4-aminopiperidine-1-carboxylate (0.112 g, 0.6 mmol), HATU (0.228 g, 0.6 mmol) and DIEA (0.244 mL, 1.5 mmol). After stirring for 30 min, the solvent was removed in vacuo to provide crude 1.5.
e. A solution of 1.5 (0.5 mmol) in TFA/CH₂Cl₂ (1:1) was stirred for 30 min. Removal of solvent in vacuo, followed by purification by reverse-phase preparatory HPLC provided the title compound 1.6.
f. To a solution of 1.6 (0.5 mmol) in DMF (5 mL) was added 2-bromoethanol (1.0 mmol), Cs₂CO₃ and NaI. After stirring at 70° C. for 4 h, the reaction solution was diluted with EtOAc and washed with water and brine. The organic layer was dried over Mg₂SO₄ and the solvent was removed in vacuo to provide crude 1.7.
g. To a solution of 1.7 (0.2 mmol) in CH₂Cl₂ (5 mL) was added MsCl (4 mmol) and TEA (1.5 mmol). After stirring at rt for 30 min, the solvent was removed in vacuo. The crude mesylate was then dissolved in DMF (2 mL), added pyrrolidine (4 mmol), Cs₂CO₃ (1.0 mmol) and NaI (0.5 mmol), and stirred for 16 h at 80° C. to provide 1.8 which was purified by reverse-phase preparatory HPLC.

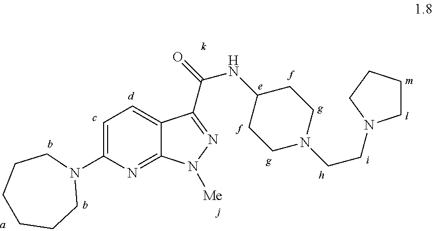

1H NMR (CDCl₃ and CD₃OD, δ ppm): 8.18 (d, 1H^{c}), 6.58 (d, 1H^{d}), 4.02 (s, 3H^{j}), 3.91-3.48 (m, 4H^{b}, 4H^{g}, 4H^{l}), 3.11-2.95 (m, 2H^{h}, 2Hⁱ), 2.80-1.95 (m, 8H^{a}, 1H^{k}), 1.82-1.72 (m, 4H^{f}), 1.49-1.47 (m, 4H^{m}). The superscripts, a-m here, and for other compounds below, correlate the resonances with the hydrogen atoms in the compound. Based on the NMR spectra provided here and/or known NMR resonances, a skilled person will be readily able to identify compounds of Formulas I, IA, IB, and others provided here, by their NMR spectra.

B. A compound of the present invention, compound 1.9 (a compound described, e.g., by Formulas IA, IB, IC, II, IIA, and IIB) was synthesized as schematically shown and described below.

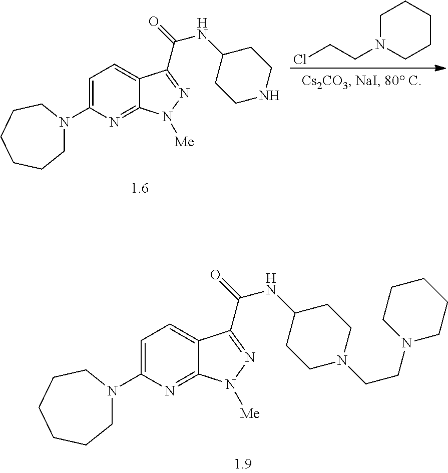

a. To a solution of 1.6 (0.1 mmol) in DMF (2 mL) was added 1-(2-chloroethyl)piperidine (0.2 mmol), Cs₂CO₃ and NaI. After stirring at 70° C. for 4 h, the reaction solution was purified by reverse-phase preparatory HPLC to provide 1.9.

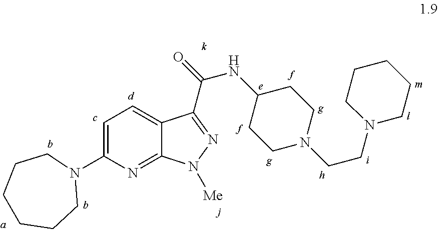

1H NMR (CDCl₃ and CD₃OD, δ ppm): 8.25 (d, 1H^{c}), 6.62 (d, 1H^{d}), 4.20 (s, 3H^{j}), 3.85 (m, 4H^{g}), 3.70-3.47 (m, 4H^{b}, 1H^{e}, 2H^{l}), 3.42 (m, 1H^{k}), 3.18 (m, 2Hⁱ), 2.92 (m, 2H^{h}), 2.19 (m, 4H^{f}), 2.00-1.82 (m, 8H^{a}, 1H), 1.58 (m, 6H^{m}).

C. A compound of the present invention, compound 1.10 (a compound described, e.g., by Formulas IA, IB, IC, II, HA, and IIB) was synthesized as schematically shown and described below.

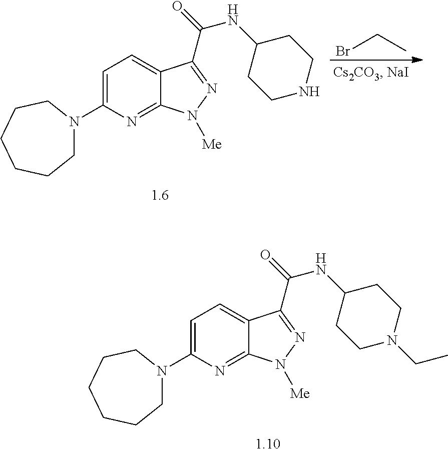

a. To a solution of 1.6 (0.1 mmol) in DMF (2 mL) was added 2-bromoethane (0.2 mmol), Cs₂CO₃ and NaI. After stirring at 70° C. for 4 h, the reaction solution was purified by reverse-phase preparatory HPLC to provide 1.10.

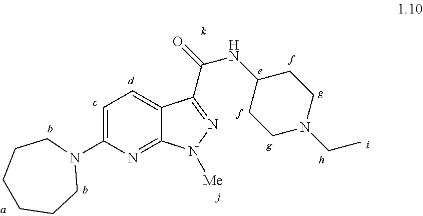

1H NMR (CDCl₃, δ ppm): 8.18 (d, 1H^{c}), 7.08 (d, 1H^{k}), 6.58 (d, 1H^{d}), 4.22 (m, H^{e}), 3.92 (s, 3H^{j}), 3.65 (m, 4H^{g}), 3.30-3.15 (m, 2H^{b}), 3.15 (q, 2H^{h}), 3.75 (m, 2H^{b}′), 2.25-2.15 (m, 4H^{f}), 1.80 (m, 4H^{a}), 1.55 (m, 4H^{a}), 1.38 (t, 3Hⁱ).

D. A compound of the present invention, compound 1.11 (a compound described, e.g., by Formulae IA, IB, IC, II, IIA, and IIB) was synthesized as schematically shown and described below.

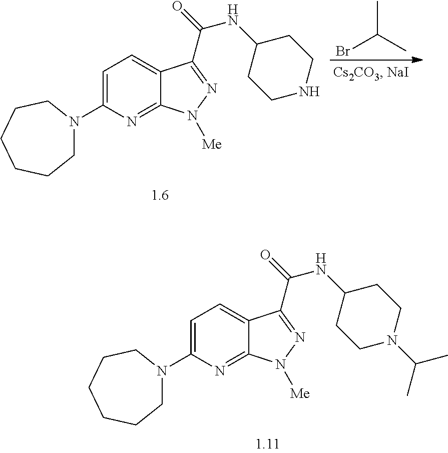

a. To a solution of 1.6 (0.1 mmol) in DMF (2 mL) was added 2-bromopropane (0.2 mmol), Cs₂CO₃ and NaI. After stirring at 70° C. for 4 h, the reaction solution was purified by reverse-phase preparatory HPLC to provide 1.11.

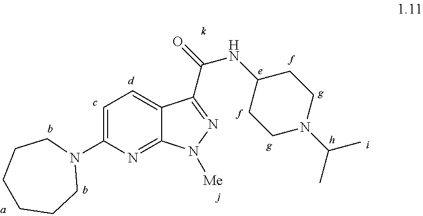

1H NMR (CDCl₃, δ ppm): 1H NMR (CDCl₃, δ ppm): 8.15 (d, 1H^{c}), 6.54 (d, 1H^{d}), 4.25 (m, 1H^{e}), 3.93 (s, 3H^{j}), 3.70 (t, 4H^{g}), 3.62-3.45 (m, 2H^{b}, 1H^{h}), 2.88 (dd, 2H^{b}′), 2.22-2.12 (m, 4H^{f}), 1.82 (m, 4H^{a}), 1.51 (m, 4H^{a}), 1.32 (d, 6Hⁱ).

E. A compound of the present invention, compound 1.12 (a compound described, e.g., by Formulae IA, IB, IC, II, HA, and IIB) was synthesized as schematically shown and described below.

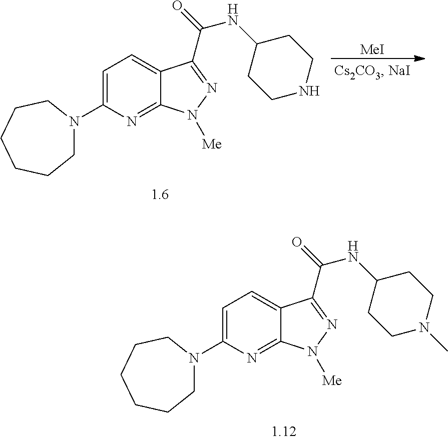

a. To a solution of 1.6 (0.1 mmol) in DMF (2 mL) was added iodomethane (0.2 mmol), Cs₂CO₃ and NaI. After stirring at 70° C. for 4 h, the reaction solution was purified by reverse-phase preparatory HPLC to provide 1.12.

F. Other compounds of the present invention, compounds 2.2 and 2.3 (described, e.g., by Formula IID), was synthesized as schematically shown and described below.

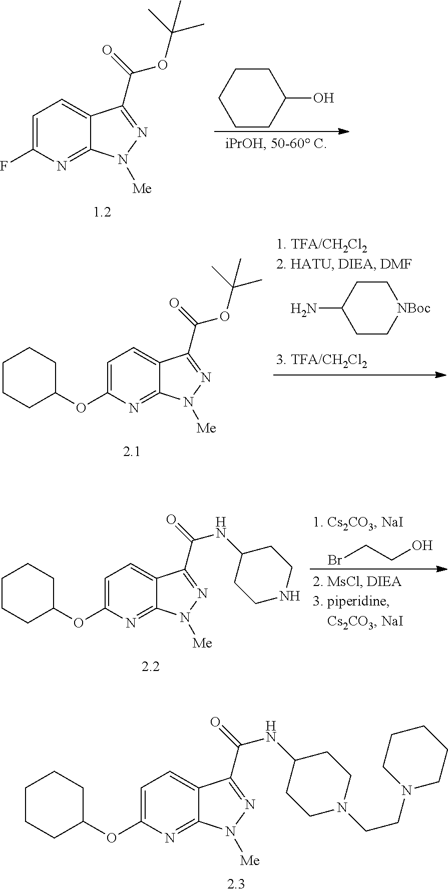

a. To a solution of 1.2 (0.5 mmol) in iPrOH was added cyclohexanol (5 mmol) and 60% NaH (5.5 mmol). After heating the solution at 50-60° C. for 12 h, the solvent was removed in vacuo to provide crude 2.1.
b. Following similar transformations as 1c-g provided 2.3.

G. Another compound of the present invention, compound 3.3 (a compound described by Formula III), was synthesized as schematically shown and described below.

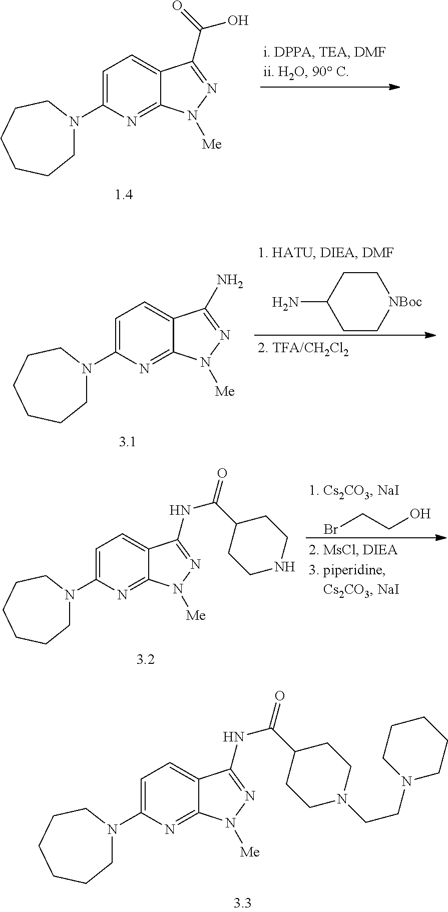

a. To a solution of 1.4 (0.080 g, 0.3 mmol) in DMF (1 mL) was added DPPA (0.136 g, 0.51 mmol) and TEA (0.050 mL, 0.51 mmol). After stirring for 1 h, water (0.1 mL) was added and the solution was heated to 90° C. for 1 h. The solution was diluted with EtOAc (10 mL) and washed with water (3×5 mL). The organic solvent was removed in vacuo to provide crude 3.1 which was used without further purification.
b. Following similar transformations as 1d-g provided 3.3.

H. Another compound of the present invention, compound 4.4 (a compound described by Formula IV), was synthesized as schematically shown and described below.

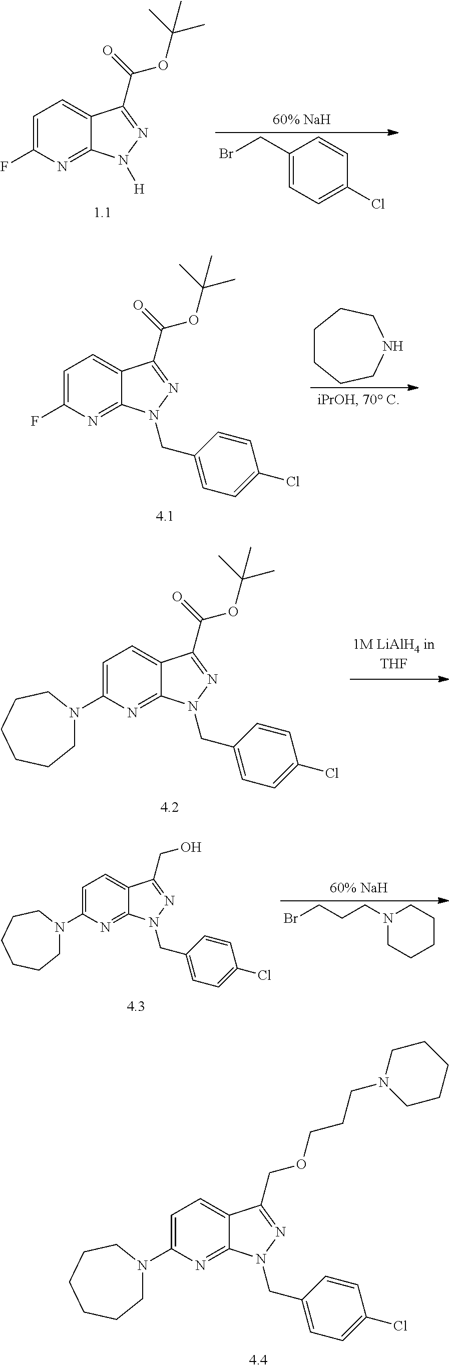

a. To a solution of 1.1 (1.0 g, 4.2 mmol) in DMF (10 mL) at 0° C. was added 60% NaH (0.169 g, 4.6 mmol). After stirring at 0° C. for 30 min, 1-(bromomethyl)-4-chlorobenzene (0.949 mL, 4.6 mmol) was added portion-wise. After stirring for 3 h, the solution was diluted with EtOAc (50 mL) and washed with saturated aqueous NaHCO₃ (3×20 mL). The organic solvents were removed in vacuo to provide crude 4.1 which was used without further purification.
b. To a solution of 4.1 (4.2 mmol) in iPrOH (50 mL) was added azepane (4.5 mmol). After heating the solution to 80° C. for 12 h, the solvent was removed in vacuo to provide crude 4.2. Purification via silica gel chromatography (10-20% EtOAc in hexanes) yielded two compounds with molecular weight corresponding to product, the higher Rf spot being the desired product 4.2.
c. To a solution of 4.2 (1.5 g, 3.4 mmol) in THF (12 mL) was added 2 M LiAlH₄ (5 mmol). After heating at 50° C. for 2 h, the solution was added dropwise 10% NaHSO₄ until no bubbles appeared. The mixture was filtered over Celite, followed by washing with THF. Removal of solvent in vacuo provided crude 4.3 which was used without further purification.
d. To a solution of 4.3 (0.1 mmol) in DMF (5 mL) was added 60% NaH (0.11 mmol). After stirring for 30 min, the solution was added 1-(3-chloropropyl)piperidine hydrochloride (0.2 mmol) and heated to 80° C. for 12 h. Purification by reverse-phase HPLC provided the title compound 4.4.

I. Another compound of the present invention, compound 5.2 (a compound described by Formula IV), was synthesized as schematically shown and described below.

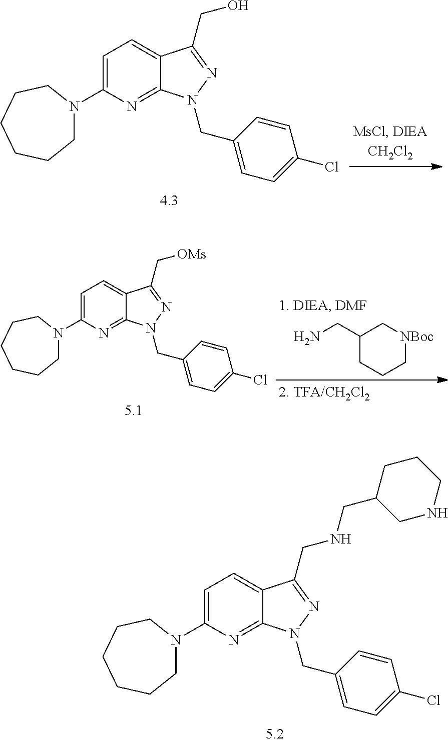

a. To a solution of 4.3 (0.4 mmol) in CH₂Cl₂ (3 mL) was added MsCl (0.039 mL, 0.5 mmol) and DIEA (0.097 mL, 0.6 mmol). After stirring for 15 min, the solvent was removed in vacuo to provide crude 5.1 which was used without further purification.
b. To a solution of 5.1 (0.13 mmol) in DMF (1 mL) was added tert-butyl 4-(aminomethyl)piperidine-1-carboxylate (0.15 mmol) and DIEA (0.2 mmol). After heating at 60° C. for 12 h, the solvent was removed in vacuo. TFA/CH₂Cl₂ (1:1) was added and stirred for 30 min. Solvent was removed in vacuo and the crude residue was purified by reverse-phase HPLC to provide title compound 5.2.

J. Another compound of the present invention, compound 23.5 (a compound described by Formula IX), was synthesized as schematically shown and described below.

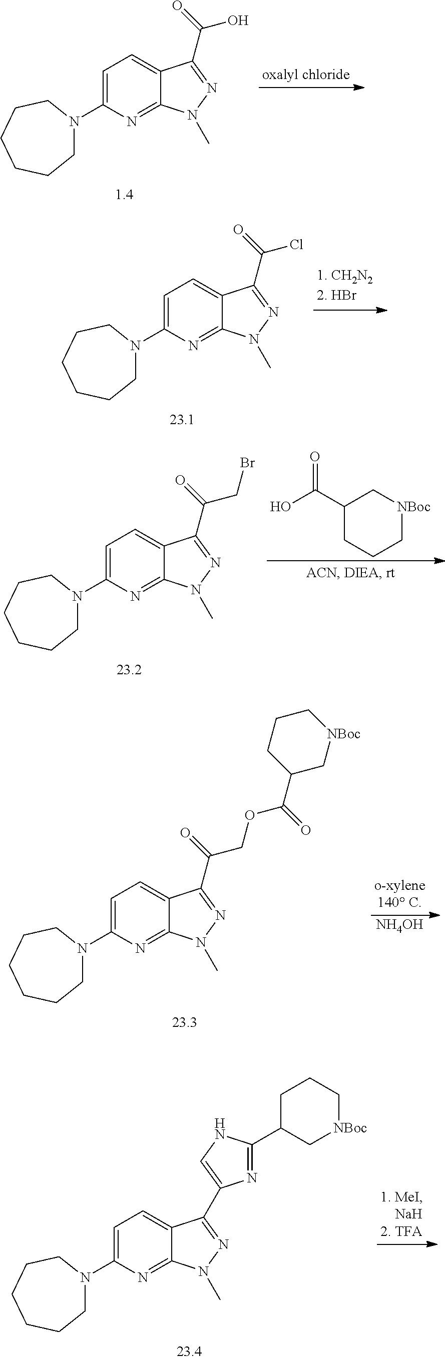

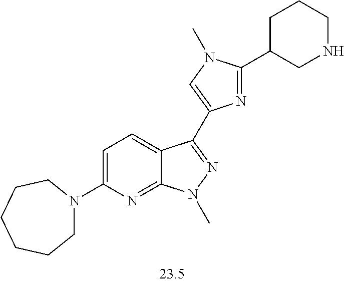

a. To a solution of 12.4 in DCM was added oxayl chloride dropwise plus 1 drop of DMF. After stirring for 1 h, the solvent was removed in vacuo. To a solution of the residue in DCM was added freshly made CH₂N₂ in ether at 0° C. After stirring for 1 h, the solvent was removed in vacuo. The residue was redissolved in HOAc. HBr (48% aqueous) was added and stirred for 30 min. The solvent was removed in, and the crude residue was dissolved in ethyl acetate and washed with aqueous NaHCO₃. The crude residue was purified via column chromatography (20% ethyl acetate in hexanes) to provide 23.2.
b. To a solution of 23.2 in ACN was added DIPEA and Boc-piperidine carboxylic acid at rt. After stirring for 15 h, the solvent was removed in vacuo, and the crude residue was dissolved in ethyl acetate and washed with aqueous NaHCO₃. The organic layer was concentrated to give 23.3 (80-90%).
c. To a solution of 23.3 in o-xylene was added NH₄OAc and Et₃N in a sealed tube. The sealed tube was heated to 140° C. for 1.5 h. The solvent was removed in vacuo, and the crude residue was dissolved in ethyl acetate and washed with aqueous NaHCO3. The organic layer was concentrated, and the crude residue was purified via column chromatography (10% MeOH in DCM) to give product 23.4 (60%).
d. To a solution of 23.4 in DCM was added TFA followed by NaH and MeI to give 23.5.

K. Another compound of the present invention, compound 24.1 (a compound described by Formula IX), was synthesized as schematically shown and described below

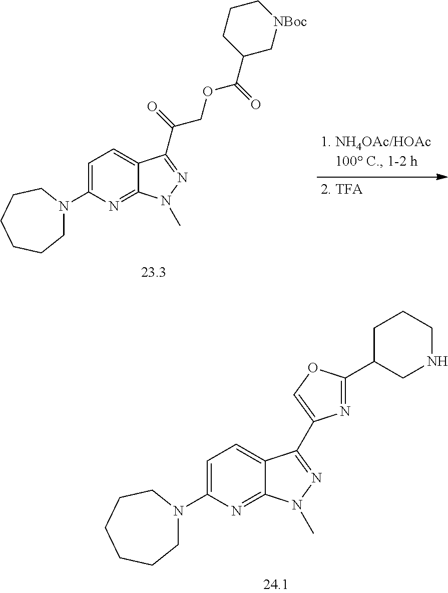

a. To a solution of 23.3 in HOAc in a sealed tube was added NH₄OAc (15 equiv) and heated to 100° C. The solvent was removed in vacuo, and the crude residue was dissolved in ethyl acetate and washed with aqueous NaHCO₃. Treatment with TFA provides 24.1.

L. Another compound of the present invention, compound 25.4 (a compound described by Formula IX), was synthesized as schematically shown and described below.

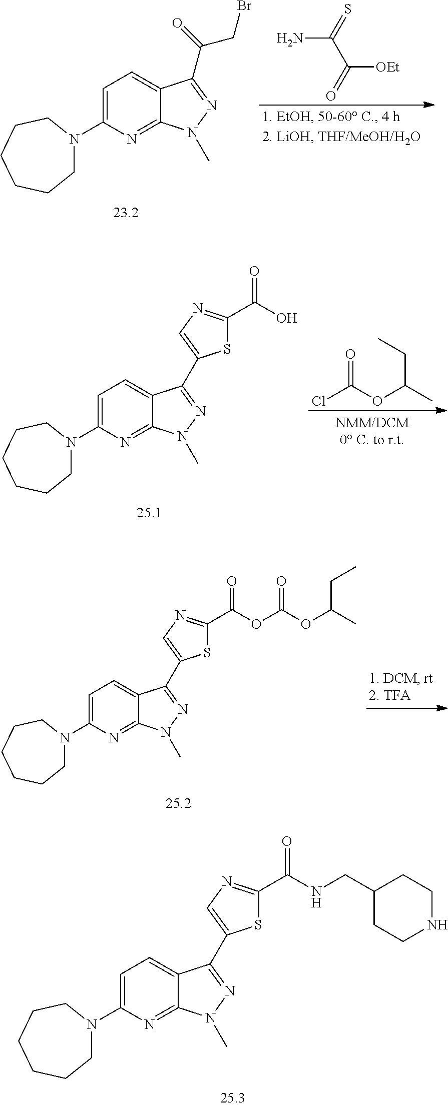

a. To a solution of 23.2 in EtOH was added ethyl thiooxamate (1.5 eq). After heating to 60° C. for 4 h the solvent was removed in vacuo, and the crude residue was dissolved in DCM and washed with aqueous NaHCO₃. The organic layer was concentrated, and the crude residue was purified via column chromatography (30% ethyl acetate in hexanes) to give 25.1. Treatment with 1 M LiOH in THF/MeOH/H₂O provides 25.1.
b. To a solution of 25.1 in DCM was added NMM and isobutyl chloroformate (2 eq) at 0° C. After stirring at rt for 1 h, the amine was added and the solution was stirred at room temperature (rt) for 1 h. The crude residue was dissolved in DCM and washed with aqueous NaHCO3. The organic layer was concentrated, and the crude residue was purified via column chromatography (10% MeOH in DCM) to give 25.2.
c. Treatment of 25.2 with TFA provides 25.3.

M. Another compound of the present invention, compound 26.3 (a compound of Formula IX), was synthesized as schematically shown and described below.

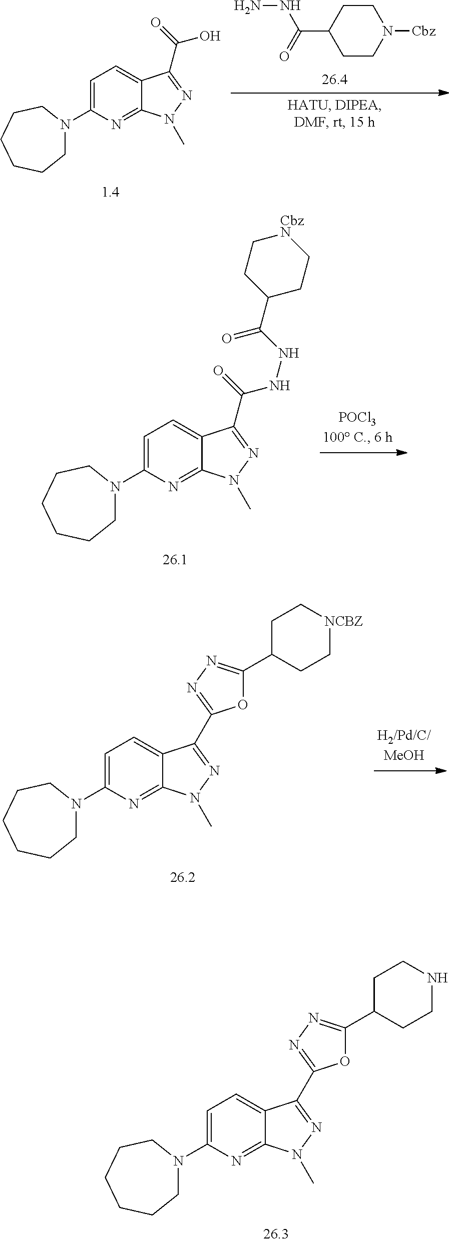

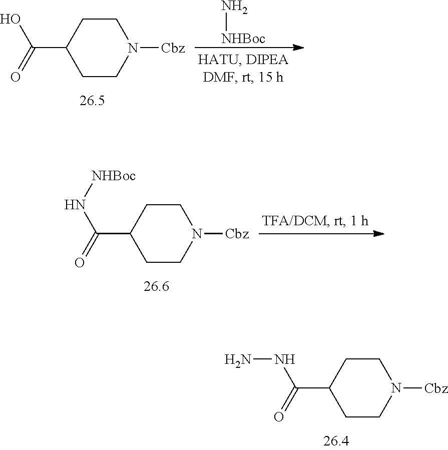

a. To a solution of 12.4 (1 equiv) in DMF was added semicarbazide, HATU (1.2 equiv) and DIPEA (1.5 equiv) to give 26.1.
b. A solution of 26.1 in POCl₃ was heated to 100° C. for 6 h. After cooling to rt, DCM was added and the solution was washed with NaHCO3. Removal of solvent provides crude product which was purified via column chromatography (10% MeOH in DCM) to provide 26.2.
c. Treatment with H₂ and Pd on carbon provides 26.3.
d. To make semicarbazide 26.4: To a solution of carboxylic acid in DMF is added HATU (1.2 equiv) and DIPEA (1.5 equiv). After stirring at rt for 15 h, the solution was diluted with DCM and washed with NaHCO₃. Removal of solvent followed by purification via column chromatography (10% MeOH in DCM) provided 26.6. Treatment with TFA provided 26.4.

N. Another compound of the present invention, compound 27.2 (a compound described by Formula IX), was synthesized as schematically shown and described below.

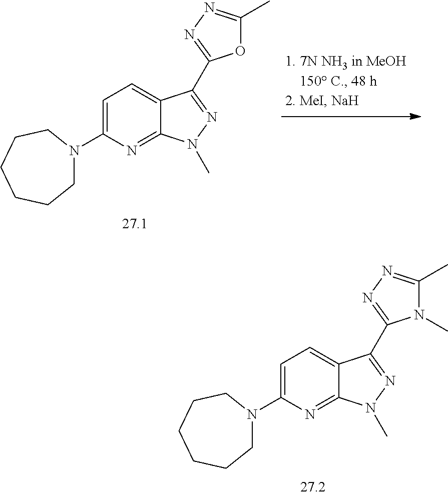

a. 27.1 was heated to 150° C. for 48 h with 7 N NH₃ in MeOH in sealed tube, followed by MeI and NaH to provide 27.2.

O. Another compound of the present invention, compound 28.2 (a compound described by Formula IX), was synthesized as schematically shown and described below.

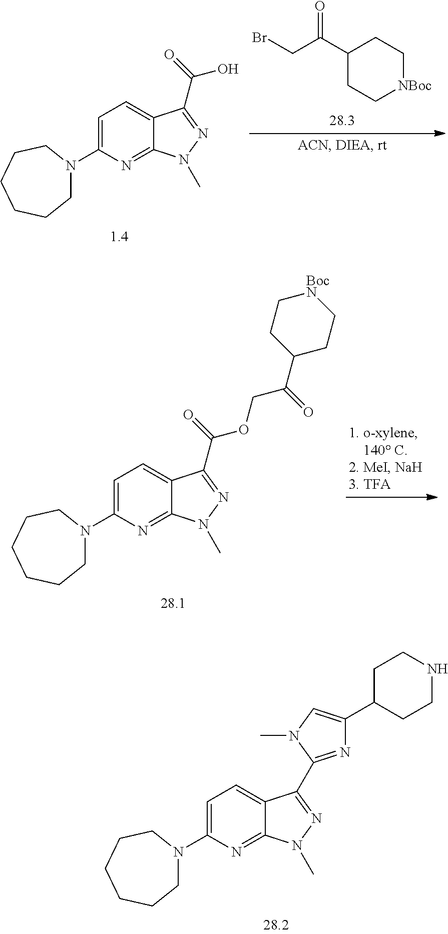

a. To a solution of 12.4 in ACN was added DIPEA and 28.3 at rt. After stirring for 15 h, the solvent was removed in vacuo, and the crude residue was dissolved in ethyl acetate and washed with aqueous NaHCO₃. The organic layer was concentrated to give 28.1.
b. To a solution of 28.1 in o-xylene was added NH₄OAc and Et₃N in a sealed tube. The sealed tube was heated to 140° C. for 1.5 h. The solvent was removed in vacuo, and the crude residue was dissolved in ethyl acetate and washed with aqueous NaHCO3. The organic layer was concentrated, and the crude residue was purified via column chromatography (10% MeOH in DCM), treated with MeI/NaH followed by TFA to give 28.2.
c. 28.3 can be made from the corresponding carboxylic acid and diazomethane followed by HBr.

P. Another compound of the present invention, compound 29.5 (a compound described, e.g., by Formula IC or IV), was synthesized as schematically shown and described below.

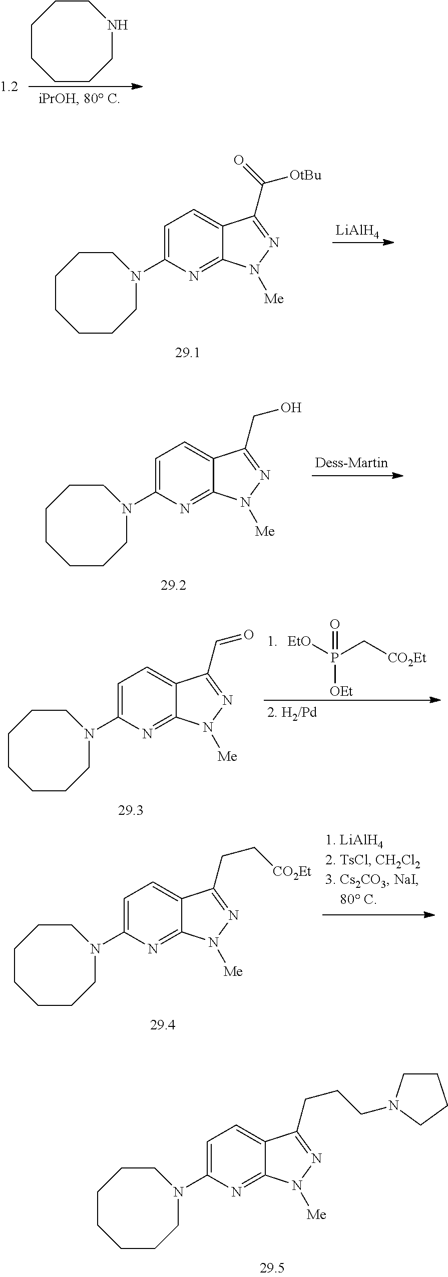

a. To a solution of 1.2 (10 mmol) in iPrOH is added azocane (30 mmol) and heated to 80° C. for 48 h. After stirring, the solvent was removed in vacuo to provide crude 29.1 which was purified via silica gel chromatography (20% ethyl acetate in hexanes) to provide pure 29.1.
b. To a solution of 29.1 (10 mmol) in THF was dropwise added 1 M LiAlH₄ in THF (30 mmol). After stirring for 16 h, saturated Na₂SO₃ in water was added dropwise until no more gas evolution was observed.
c. To a solution of 29.2 (10 mmol) in CH₂Cl₂ was added Dess-Martin reagent and stirred for 1 h.
d. To a solution of ethyl 2-(diethoxyphosphoryl)acetate (11 mmol) in THF at 0° C. was added NaH (11 mmol). After stirring for 1 h, a solution of 29.3 (10 mmol) in THF is added, and the reaction mixture was stirred for 2 h at rt.
e. To a solution of 29.4 (5 mmol) in EtOH was added Pd on C and placed under an atmosphere of H₂ via balloon for 16 h.
f. To a solution of 29.4 (5 mmol) in THF was added 1 M LiAlH₄ (10 mmol) in THF. The alcohol (4 mmol) was then treated with TsCl (4.4 mmol) and DIEA (8 mmol) to provide the tosylate. The reaction solution was diluted with CH₂Cl₂ and washed with water. The organic layer was dried and solvent was removed in vacuo to provide crude tosylate. Crude tosylate was dissolved in DMF and heated to 80° C. with pyrrolidine, Cs₂CO₃ and NaI overnight to provide 29.5 which was purified via reverse-phase prepatory chromatography.

### 2. Testing for Anti HCV Genotype 1b Activity of the Compounds of the Present Invention

A suitable 1b HCV RNA replicon assay uses the Huh7 cell line which contains an HCV 1b RNA replicon with a stable luciferase (LUC) reporter. This construct contains modifications that make the cell line more robust and provides stable LUC expression for antiviral screening. The LUC reporter is used as an indirect measure of HCV replication. The activity of the LUC reporter is directly proportional to HCV RNA levels and positive control antiviral compounds behave comparably using LUC endpoints.

HCV assays suitable for use in demonstrating the anti-viral activity of the compounds useful in the methods of the invention include the Luciferase Assay for HCV Replicon Reporter Cell Lines and the MTT Assay for HCV Replicon Reporter Cell Lines described in this example. The embodiments of these assays described in this example were developed by Shanghai ChemPartner Co., Ltd., a corporation of China with its principal office located at 720 Cailun Road, Building No. 3, Shanghai 201203, China.

### A. Luciferase Assay for HCV Replicon Reporter Cell Lines

Fresh growth medium is prepared just before use. The container used in the procedure is a 10 cm diameter culture dish. HCV replicon reporter cell lines are used. Prepare complete medium: add FBS and appropriate additives as described in “Culture Media”, below. Pre-warm the medium in a 37° C. thermostat water bath. Remove the dish from a 37° C. CO₂ incubator. Check the cell name and complete medium and passage number marked on the dish. Aspirate the medium carefully and add 1 ml PBS to rinse the cells. Remove and discard the solution and add 1 ml of 0.25% Trypsin/0.02% EDTA. Rinse the cells with the added Trypsin/EDTA to ensure all the cells have been rinsed. Remove the Trypsin/EDTA with a vacuum pump and incubate at 37° C. for 3-5 minutes. Examine the cell morphology under an inverted microscope to confirm that a single cell suspension is clearly visible. Add 3 ml of complete medium to the dish and suspend the cell by gentle pipetting. Count the cell numbers with a hematometer. Adjust cell density to 100 k/ml by adding appropriate volume of the complete medium. Add 100 μl of cell suspension to each well of a 96-well white plate; the cell density is thus 10 k per well. Mark the plate with cell name, passage number, seeding density, date and the name of the operator. Place the 96-well assay plate in 37° C. 5% CO₂ incubator for 24 hours.

Compounds are prepared or provided at 25 mM in 100% DMSO. This is the compound stock solution. The dilution procedure should be performed in a cell culture hood. Dispense the stock solution into the second column of a 96-well plate. Prepare 9-step (10 concentrations total), 5-fold serial dilutions by transferring 10 μA of the compound into the next well containing 40 μA of DMSO. Repeat for all compounds. Aspirate 2 μl of the above compound solution from each well and add into 198 μl complete media using a 12-channel pipetter to obtain the 10-fold concentration compound solution with 1% DMSO, mix well.

Remove the 96-well assay plate from the 37° C./5% CO₂ incubator, examine the cell morphology under an inverted microscope. In a cell culture hood, add 10 μl of the 10× concentration compounds solution into each well on the 96-well assay plate. All compound's dose responses are done in duplicate. The starting final concentration of the compounds is 25 μM, and DMSO final concentration 0.1%. Mark the plate with compound code(s) and concentrations. Place the 96-well assay plate into CO₂ incubator for 48 hours. Add 30 μl of Stead-Glo Luciferase System (Promega) reagent to each well and mix by gentle shaking on a plate shaker for 5 minutes to allow throughout cell lysis. Measure the luminescence with Envision (Perkin Elmer) with an integration time of 2 seconds. Record and analyze the data.

The cell culture media is DMEM complete: DMEM (Life Technologies #41965-039) supplemented with 10% FCS, 2 mM Glutamin (Life Technologies #25030-024), Penicillin (100 IU/ml)/Streptomycin (100 μg/ml) (Life Technologies #15140-114) and 1× nonessential amino acids (Life Technologies #11140-035). G418 (“Geneticin”, Life Technologies): concentrations are given as weight per volume of the original substance. Specific activity of a typical batch is ca. 700 μg/mg as stated by the manufacturer. This value does not necessarily reflect the biological activity in a user's system. Therefore each new batch of G418 should be tested individually e.g. in an electroporation experiment using different selection conditions (0.2-1 mg/ml).

### B. MTT Assay for HCV Replicon Reporter Cell Lines

The MTT assay (and the MTS assay) is a laboratory test and standard colorimetric assay (an assay which measures changes in color) for measuring the activity of enzymes that reduce MTT or MTS+PMS to formazan, giving a purple color. It can also be used to determine cytotoxicity of potential medicinal agents and other toxic materials, since those agents would result in cell toxicity and therefore metabolic dysfunction and therefore decreased performance in the assay. Yellow MTT (3-(4,5-dimethylthiazol-2-yl)-2,5-diphenyltetrazolium bromide, a tetrazole) is reduced to purple formazan in living cells. A solubilization solution (usually either dimethyl sulfoxide, an acidified ethanol solution, or a solution of the detergent sodium dodecyl sulfate in diluted hydrochloric acid) is added to dissolve the insoluble purple formazan product into a colored solution. The absorbance of this colored solution can be quantified by measuring at a certain wavelength (usually between 500 and 600 nm) by a spectrophotometer. The absorption maximum is dependent on the solvent employed.

Culture medium, culture plates, and additives are prepared as described in part A of this example. Pre-warm the medium in a 37° C. thermostat water bath. Remove the dish from a 37° C. CO₂ incubator. Check the cell name and complete medium and passage number marked on the dish. Aspirate the medium carefully and add 1 ml PBS to rinse the cells. Remove and discard the solution and add 1 ml of 0.25% trypsin/0.02% EDTA. Rinse the cells with the added Trypsin/EDTA to ensure all the cells have been rinsed. Remove the trypsin/EDTA with a vacuum pump and incubate at 37° C. for 3-5 minutes. Examine the cell morphology under an inverted microscope until single cell suspension is clearly visible. Add 3 mL of complete medium to the dish and suspend the cell by gentle pipetting. Count the cell numbers with a hematometer. Adjust cell density to 100 k/ml by adding appropriate volume of the complete medium. Add 100 μl of cell suspension to each well of a 96-well white plate; the cell density is thus 10 k per well. Mark the plate with cell name, passage number, seeding density, date and the name of the operator. Place the 96-well assay plate in 37° C. 5% CO₂ incubator for 24 hours.

Compounds are prepared or provided at 25 mM in 100% DMSO. This is the compound stock solution. The dilution procedure should be performed in a cell culture hood. Dispense the stock solution into the second column of a 96-well plate. Prepare 9-step (10 concentrations total), 5-fold serial dilutions by transferring 10 μl of the compound into the next well containing 90 μl of DMSO. Repeat for all compounds. Aspirate 2 μl of the above compound solution from each well and add into 198 μl complete media using a 12-channel pipetter to obtain the 10-fold concentration compound solution with 1% DMSO, mix well. Remove the 96-well assay plate from 37° C./5% CO₂ incubator, examine the cell morphology under an inverted microscope. In a cell culture hood, add 10 μl of the 10× concentration compound solution into each well on the 96-well assay plate. All compound's dose responses are done in duplicate. The starting final concentration of the compounds is 25 μM, and DMSO final concentration 0.1%.

Mark the plate with compound code(s) and concentrations. Place the 96-well assay plate into CO₂ incubator for 48 hours. Add 10 μl of 5 mg/ml MTT to each well and incubate in the 37° C. CO₂ incubator for 4 hours. Add 100 μl of testing solution (10% SDS+5% isobutyl alcohol+10 mmol/L HCl) to each well directly and incubate in the 37° C./5% CO₂ incubator overnight. Measure the absorbance at 580/680 nm on SpectraMax Plus 384 (MDC). Record and analyze the results.

### 3. HCV Genotype 2a Infectious Clone Assay

In an embodiment, the replication assay protocol can include the following stages. It should be noted that the following replication assay protocol is non-limiting, and is presented as an illustrative embodiment of a replication assay protocol. The assays in parts A and B, above, were used to generate the genotype 1b inhibitory activity and related cell toxicity (viability) data. The assay below can be used to generate genotype 2a inhibitory activity data.

### Stage 1: RNA Transcription

1) Linearize the FL-J6/JFH-5′C19Rluc2AUbi plasmid with XbaI at 37° C. for 2 hrs, and run on 1% agarose gel to check completeness of digestion. 2) Digest the 5′ overhangs by treatment with mung bean nuclease at 30° C. for 30 min. 3) For linearization of the Bart79I-luc plasmid (similar to Bart79I plasmid as described in Elazar et al. J. Virol. 2003, 77(10):6055-61 except that the neomycinphosphotransferase gene has been replaced with the gene encoding firefly luciferase) use ScaI restriction endonuclease, then examine the linearized template DNA on a gel to confirm that cleavage is complete, follow this with proteinase k digestion. 4) Purify templates by digestion with proteinase K for 30 min, phenol-chloroform extraction, ethanol precipitation, and then resuspend at 1 μg/μl. 5) For the transcription reaction, use 1 μg of purified template by using the T7 Megascript kit for FL-J6/JFH-5′C19Rluc2AUbi (Ambion, Austin, Tex.) or the RiboMax™ kit for Bart79I-luc (Promega, Madison, Wis.). Incubate reactions at 37° C. for 4 h. 6) Add DNAse for 15 min. 7) Extract with an equal volume of phenol/chloroform and then with an equal volume of chloroform. Recover aqueous phase and transfer to new tube. 8) Precipitate the RNA by adding 1 volume of isopropanol and mixing well. 9) Chill the mixture for at least 15 min at −20° C. Centrifuge at 4° C. for 15 min at maximum speed to pellet the RNA. 10) Carefully remove the supernatant solution and resuspend the RNA in RNase/DNase-free Water at 1 μg/μl. 11) Run on a gel and check RNA concentration. 12) Make aliquots and store in −80° C.

### Stage 2: Electroporating Huh7.5 Cells

1) Wash cells once with PBS, trypsinize 2) Resuspend cells in a total volume of 5 ml per 10 cm plate of complete medium (pull all together) in 50 ml tubes. 3) Pellet cells at 1000×RPM for 5 min at 4° C. Aspirate supernatant and resuspend in 10 ml ice cold RNAse free filtered 1×PBS (BioWhitaker)—pipette up and down ˜5 times gently to get rid of cell clumps. 4) Pellet cells again at 1000×RPM as before and again resuspend in 10 ml ice cold PBS (BioWhitaker). 5) Remove a 10 μl aliquot to determine cell concentration. 6) Pellet cells again and resuspend in a final concentration of 1.5×10⁷ cells/ml in ice cold RNAse free-PBS. Need: 6×10⁶ cells in 0.4 ml per each electroporation (ep) and 5 μg of FL-J6/JFH-5′C19Rluc2AUbi RNA or Bart79I-luc RNA. 7) Place 5 μg RNA aliquot in an eppendorf tube (1 tube per ep). 8) Remove 0.4 ml of the cell suspension and add to the RNA. Mix twice by pipeting. 9) Immediately transfer 0.4 ml to a 2 mm gap ep cuvette. 10) Pulse the cells: 820v, 5 pulses, 99 μsec, 220 ms interval, unipolar. 11) Allow cells to rest for 15 min. 12) Transfer cells using the Pasteur pipette in the cuvette package to medium. Make a common stock from all tubes. 13) Plate 10,000 cells/well in 96 well plates. 14) Rotate plate a little for even cell plating. 15) Incubate for 24 hr before treatment.

### Stage 3: Treating Plates

1) About 24 hr following electroporation prepare medium with the desired concentration of the drug. 2) Aspirate the medium and add 100 μl of fresh medium and drug. Leave untreated wells at the beginning and again at the end. 3) Repeat daily for 2 more days.

Stage 4: Harvesting (day 5 from electroporation)

1) Alamar blue assay—a) Include medium for background subtraction (and also for seeing change in color easily). b) Aspirate medium. c) Make a stock of medium plus 10% Alamar blue. Total volume per well is 100 μl. d) Incubate for 2-2.5 hrs at 37° C. (or until there is a color change). c) Read plates at flex station.

2) Renilla Luciferase assay—a) Aspirate medium with Alamar blue. b) Wash with 1×PBS. c) Aspirate completely (aspirate, then tilt and aspirate remainders of buffer again). d) Make sure which lysis buffer is needed: firefly or renilla. e) Add 30 μl of 1× lysis buffer (add 1 volume of 5× lysis buffer to 4 volumes of sterile water). f) Shake the plate for 15 min. g) Freeze at −80° C. At this point, one can stop or continue to the next phase.

Stage 5: Reading by Luminometer. a) Thaw the plate. b) Leave plate on ice until ready to read. c) Prepare substrate reagent you need; for the renilla: thaw renilla buffer, make 1 volume 100× Renilla luc substrate plus 100 vol luc assay buffer+2 ml for priming luminometer (e.g., for 4 ml Renilla lucsubstrate, add 40 ul assay buffer). For the firefly; thaw 10 ml firefly buffer and add to the luciferase reagent. d) Read plates using a standard luminometer according to the manufacturer's directions.

### 4. HERG Channel Assay

Drugs have been shown to be associated with QT prolongation and in some cases serious ventricular arrhythmias. The most common mechanism for these adverse events is the inhibition of one or more cardiac potassium channels, in particular hERG. This current is important for cardiac myocyte repolarization and is a common target for drugs that prolong the QT interval. Test articles in this study were therefore characterized to determine their ability to inhibit the hERG channel. Ion channel activity was measured using a stably transfected Chinese Hamster Ovary (CHO) cell line expressing the hERG mRNA. The pharmacology of this cloned channel expressed in the CHO cell line is very similar to that observed in native tissue.

Cells: AVIVA's CHO cell line, which stably expresses hERG channels, was used for the study. Cells were cultured in DMEM/F12 containing 10% FBS, 1% penicillin/streptomycin and 500 μg/ml G418. Before testing, cells were harvested using Accumax (Innovative Cell Technologies).

Solutions: For electrophysiological recordings, the following solutions were used. External Solution: 2 mM CaCl₂; 2 mM MgCl₂; 4 mM KCl; 150 mM NaCl; 10 mM Glucose; 10 mM HEPES; 310-320 mOsm; pH 7.4 (adjusted with 1M NaOH). Internal Solution: 140 mM KCl; 10 mM MgCl₂; 6 mM EGTA; 5 mM HEPESNa; mM ATP-Mg; 300-320 mOsm; pH 7.25 (adjusted with 1M KOH).

Electrophysiology: Whole cell recordings were performed using PX 7000A (Axon Instruments) with VIVA's SealChip™ technology. Cells were voltage clamped at a holding potential of −80 mV. The hERG current was then activated by a depolarizing step to −50 mV for 300 ms. This first step at −50 mV was used as a baseline for measuring peak amplitude of the tail current. Next, a voltage step to +20 mV was applied for 5 s to activate the channels. Finally a step back to −50 mV for 5 seconds removed activation and the deactivating tail current was recorded.

Compound Handling and Dilutions: All compounds were prepared from either 10 or 30 mM DMSO stock solutions. Solutions were mixed by sonication for 20 min, followed by vigorous vortexing. Prior to testing, compounds were diluted to test concentrations in glass vials using External Solution. Dilutions were prepared no longer than 20 min prior to use. Equal amounts of DMSO (0.1%) were present in all final dilutions.

Electrophysiology Procedures: After achieving whole cell configuration, cells were monitored for 90 s to assess stability and then washed with External Solution for 66 s. The voltage protocol described above was then applied to the cells every 12 s throughout the procedure. Only stable cells with recording parameters above threshold (see Quality Control section) were allowed to enter the drug addition procedure. External solution containing 0.1% DMSO (vehicle) was applied to the cells to establish a baseline. After allowing the current to stabilize for 3 to 5 min, test articles were applied. Test article solutions were added to cells in 4 separate additions. Cells were kept in test solution until effect of the test article reached steady state, to a maximum of 12 min. Next, 1 μM cisapride (positive control) was added. Finally, washout with External Solution was performed until the recovery current reached a steady state.

Data Analysis: Data analysis was performed using DataXpress (Axon Instruments), Clampfit (Axon Instruments) and Origin (Originlab Corporation) software.

Quality Control: Data included in the report originated from experiments that satisfied all of the following criteria: a) Recording Parameters: membrane resistance (Rm): >200 MΩ; access resistance (Ra): <15MΩ; tail current amplitude: >150 pA; b) Pharmacological Parameters: 1 μM cisapride: >95% inhibition.

### 5. Synergy Studies

Combination studies were conducted in the 1b replicon assay, combining at least two compounds at several concentrations (below, at, and above its EC₅₀ value in the 1b replicon assay). Luciferase values were then analyzed using MacSynergy™ to determine whether the affects of the drug combinations were strongly synergistic (synergy volume>100), moderately synergistic (50<synergy volume<100), mildly synergistic (25<synergy volume<50), additive (−25<synergy volume<25), mildly antagonistic (−50<synergy volume<−25), moderately antagonistic (−100<synergy volume<−50) or strongly antagonistic (synergy volume<−100). To illustrate, see combinations of EBP1047 and clemizole (EBP1) (Entry 15) in the table below, which shows a synergy volume of 495 μM², indicating strong synergism in genotype 1b.

Additional synergistic combinations between clemizole (EBP1) and other aza-indazole analogs were identified as exemplified in entries 5 (EBP1 and EBP697), 8 (EPB1 and EBP726), 10 (EBP1 and EBP756), and 12 (EBP1 and EBP841), 13 P909), 14 (EBP1 and EBP987), 18 (EBP1 and EBP 1147), 19 (EBP1 and EBP1171), 20 (EBP1 and EBP1452), 21 (EBP1 and EBP1456), 22 (EBP1 and EBP1479) and 23 (EBP1 and EBP1489). EBP697, EBP726, EBP756, EBP841, EBP909, EBP987, EBP1147, EBP1171, EBP1452, EBP1456, EBP1479, and EBP1489. have EC₅₀ values less than 3 μM in the 48 hour 1b replicon assay.

In the table or results below, EBP520 is boceprevir, EBP521 is ITMN-191 (also known as RG7227).

TABLE 2

| | | | Mac | |
|---|---|---|---|---|
| | | | Synergy | |
| | Combo | | Results | |
| | Cmpd #1 | Cmpd #2 | (μM²) | Comments |
| 1 | 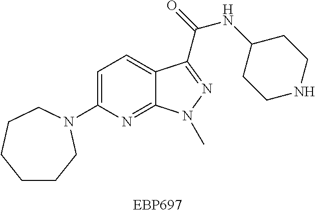 | 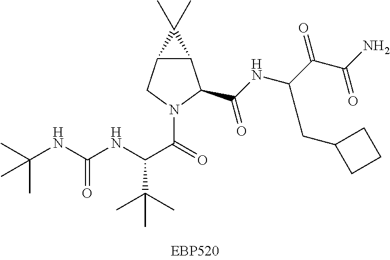 | −75 | Moderate antagonism |
| 2 | 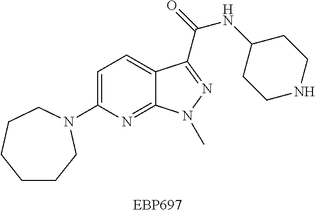 | 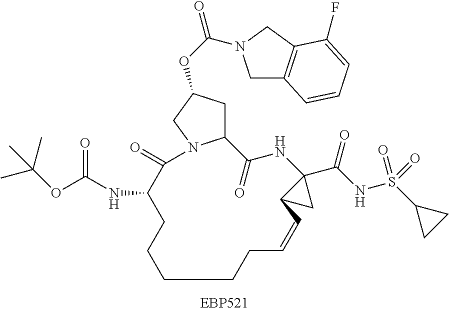 | −35 | Additivity |
| 3 | 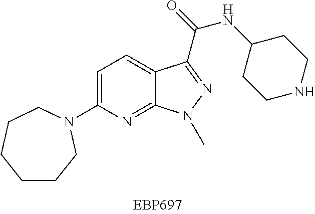 | 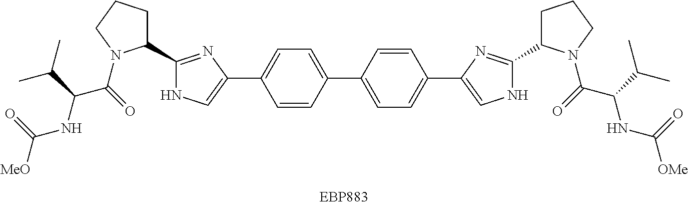 | −221 | Significant antagonism |
| 4 | 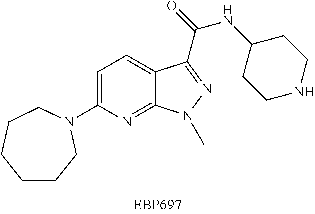 | 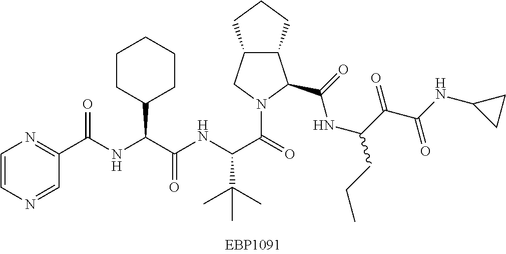 | −78 | Moderate antagonism |
| 5 | 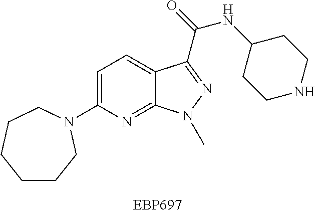 | 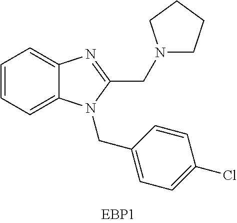 | 234 | Strong synergy |
| 6 | 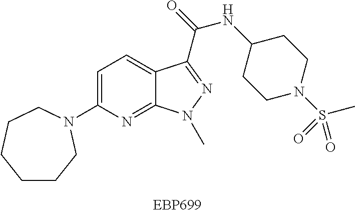 | 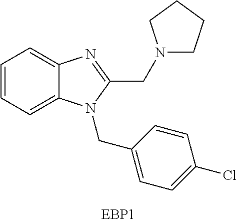 | 49 | Minor synergy |
| 7 | 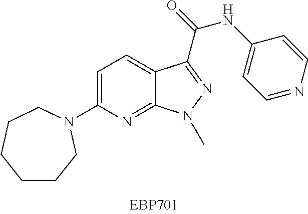 | 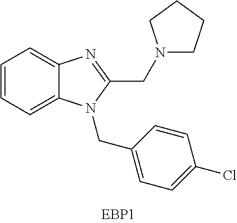 | 40 | Minor synergy |
| 8 | 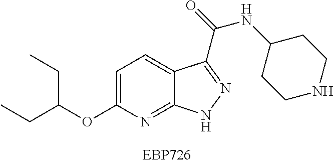 | 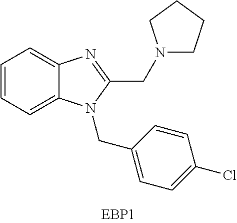 | 153 | Strong synergy |
| 9 | 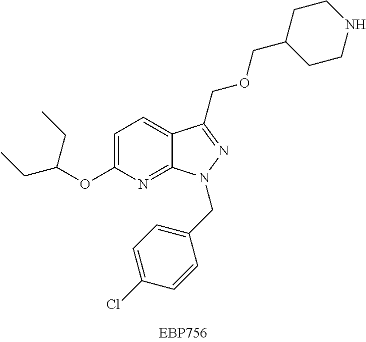 | 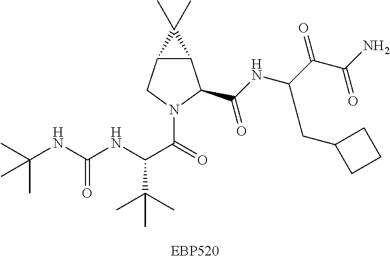 | −164 | Strong antagonism |
| 10 | 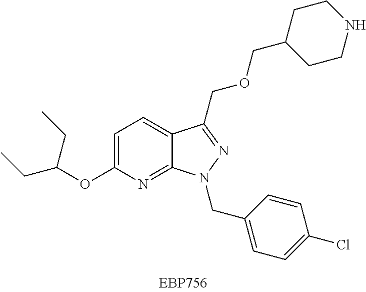 | 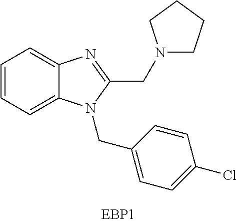 | 144 | Strong synergy |
| 11 | 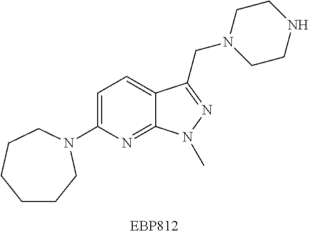 | 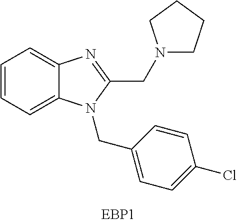 | 33 | Minor synergy |
| 12 | 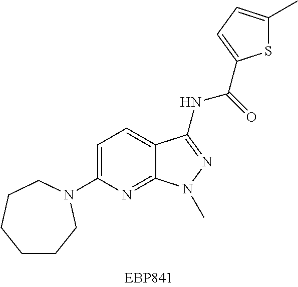 | 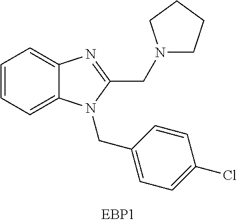 | 77 | Moderate synergy |
| 13 | 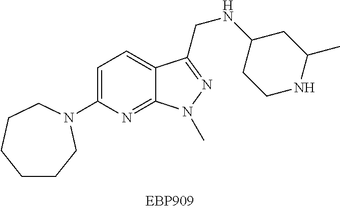 | 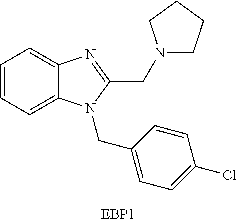 | 587 | Strong synergy |
| 14 | 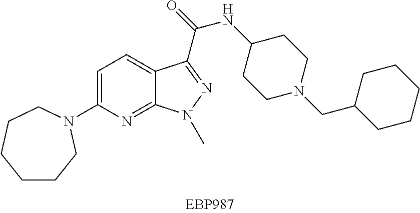 | 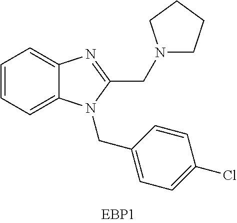 | 153 | Strong synergy |
| 15 | 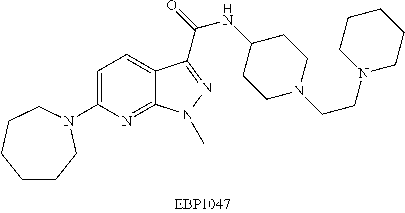 | 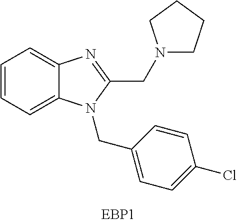 | 495 | Strong synergy |
| 16 | 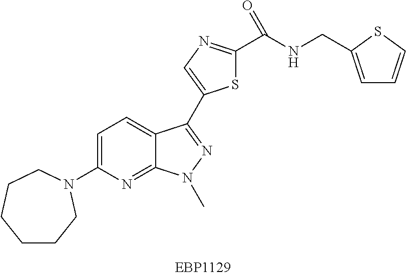 | 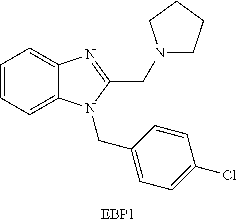 | −2 | Additivity |
| 17 | 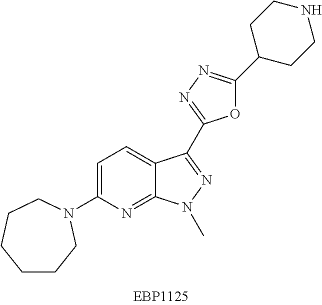 | 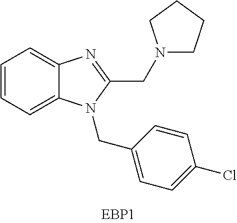 | 29 | Additivity |
| 18 | 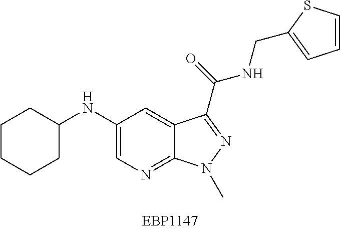 | 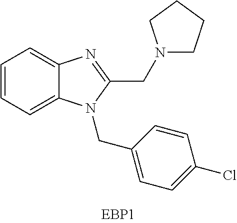 | 51 | Moderate synergy |
| 19 | 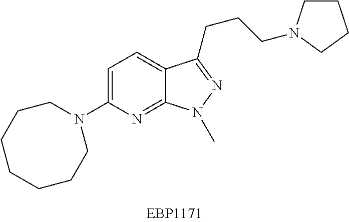 | 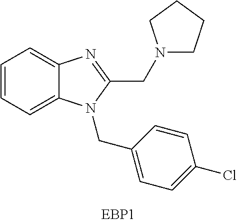 | 299 | Strong synergy |
| 20 | 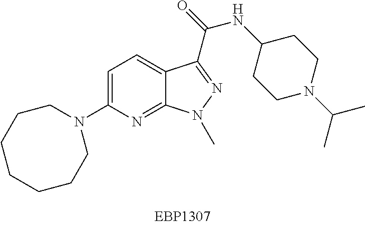 | 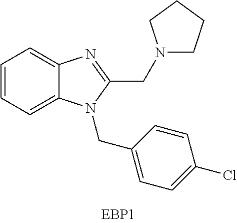 | 115 | Strong synergy |
| 21 | 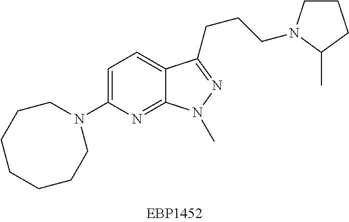 | 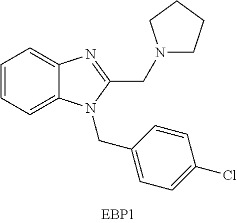 | 405 | Strong synergy |
| 22 | 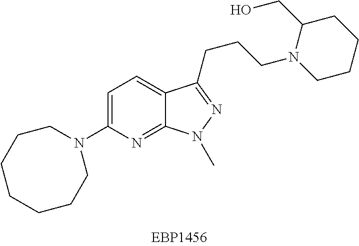 | 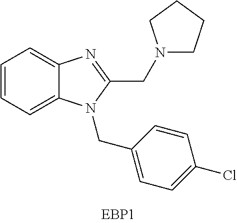 | 267 | Strong synergy |
| 23 | 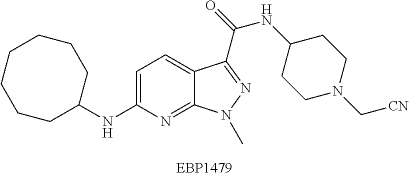 | 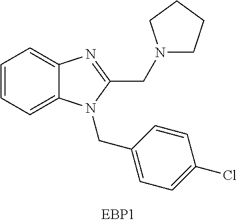 | 363 | Strong synergy |
| 24 | 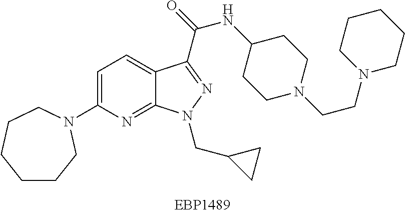 | 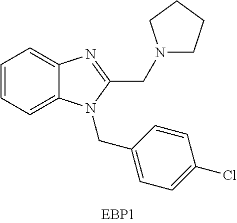 | 207 | Strong synergy |
| 25 | 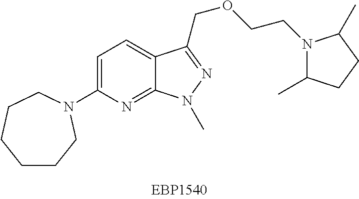 | 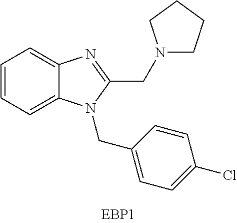 | 352 | Strong synergy |
| 26 | 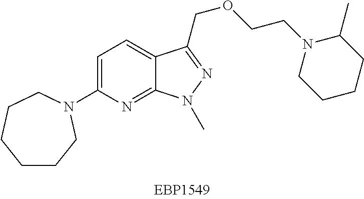 | 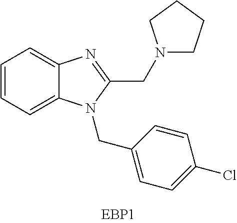 | 391 | Strong synergy |
| 27 | 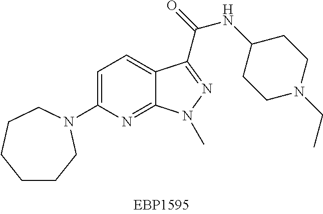 | 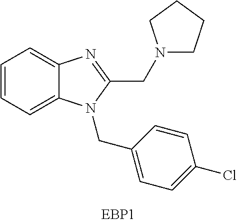 | 20 | Additivity |
| 28 | 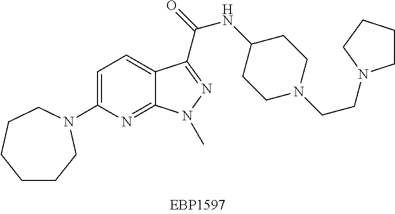 | 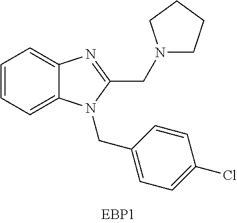 | 92 | Moderate synergy |

### 6. Caco-2 Permeability Testing

Intestinal epithelium permeability is a critical characteristic that determines the rate and extent of human absorption and affects bioavailability of a drug candidate. Poor intestinal permeability leads to limited absorption. Generally, higher absorption is preferred. The Caco-2 cell line is a human colon adeno-carcinoma cell line that resembles the epithelial lining of the human small intestine. Several transport proteins that are expressed in human intestinal epithelium are also expressed in the Caco-2 cell model. Some transporter proteins are efflux systems which mediate the secretion of compounds from inside the cell back to the apical lumen (representative of the intestinal lumen), limiting overall absorption. In a permeability assay, the apparent permeability coefficient is measured between the apical to basolateral sides of a cell monolayer, Papp (A-B), and the basolateral to the apical side, Papp (B-A). Papp (A-B)<2×10⁻⁶ cm/s is predictive of low permeability to the basolateral side, 2×10⁻⁶ cm/s<Papp (A-B)<20×10⁻⁶ cm/s is predictive of medium permeability, and Papp (A-B)>20×10⁻⁶ cm/s is predictive of high permeability. In the B-A permeability assay, Papp (B-A) is measured and compounds evaluated for efflux potential. Together, these assay results are reported as a ratio of Papp (B-A) to Papp (A-B). Papp (B-A)/Papp (A-B)>3 are likely to be transported by one of the efflux systems, and have poor absorption. Papp (B-A)/Papp (A-B)<3 are likely to show reasonable intestinal absorption. An in vitro drug absorption assay suitable for use in predicting intestinal absorption of the compounds useful in the methods of the invention is described in this example. The embodiments of this assay described in this example were developed by Shanghai ChemPartner Co., Ltd., a corporation of China with its principal office located at 720 Cailun Road, Building No. 3, Shanghai 201203, China.

### Reagents

Caco-2 (ATCC, catalog No. HTB-37™) cells are cultured in growth medium (MEM+10% FBS+1% NEAA). The growth medium is prepared by adding 50 mL FBS and 5 mL NEAA to 445 mL of MEM or by adjusting the final volume according to actual needs. Trypsin-EDTA (Invitrogen, Cat #25200-072) is also used.

### Assay and Dosing Solution Buffer

Hanks Balanced Salt Solution (HBSS, Invitrogen, Cat #14025-092) with 25 mM HEPES, pH 7.4, HBSS buffer with 0.2% DMSO (50 μL DMSO into 25 ml HBSS buffer), and HBSS buffer with 0.4% DMSO (100 μL DMSO into 25 ml HBSS buffer) are used.

### Controls

10 mM stock solutions of a compound is prepared in DMSO and Lucifer Yellow in assay buffer using the formula (Actual Weight/Molecular Weight)/mL solvent=10 mM. Erythromycin, Metoprolol, and Atenolol are used as reference compounds. Donor solutions (10 μM for compounds and 5 μM for Lucifer Yellow) are prepared. For A-B, 4 μL of 10 mM compound stock solution and 2 μL of 10 mM Lucifer Yellow stock solution are loaded into 4 mL of HBSS buffer, and centrifuged (5 min, 4000 rpm) to precipitate undissolved particles. Supernatants are collected for compound dosing. For B-A, 4 μL (compounds) of 10 mM compound stock solution is added to 4 mL HBSS buffer with 0.2% DMSO, and centrifuged (5 min, 4000 rpm) to precipitate undissolved particles. Supernatants are collected for compound dosing.

Receiver solutions are prepared. For A-B, the solution is HBSS buffer with 0.4% DMSO. For B-A, 2 μL of 10 mM Lucifer Yellow stock solution is added to 4 mL of HBSS buffer with 0.2% DMSO. Compound solutions are prepared for standard curve (3 μM/1 μM/0.2 μM/0.04 μM/0.01 μM/0.005 μM):

20× solution:
15 μL (10 mM)+485 μL (MeOH:H₂O=1:1) - - - 500 μL (300 μM)

### 200 μL (300 μM)+800 μL (MeOH: H₂O=1:1) - - - 1000 μL (60 μM)

### 200 μL (60 μM)+400 μL (MeOH: H₂O=1:1) - - - 600 μL (20 μM)

### 200 μL (20 μM)+800 μL (MeOH: H₂O=1:1) - - - 1000 μL (4 μM)

### 200 μL (4 μM)+800 μL (MeOH: H₂O=1:1) - - - 1000 μL (0.8 μM)

### 200 μL (0.8 μM)+600 μL (MeOH: H₂O=1:1) - - - 800 μL (0.2 μM)

### 200 μL (0.2 μM)+200 μL (MeOH: H₂O=1:1) - - - 400 μL (0.1 μM)

1× solution: 3 μL of 20× solution (0.1-60 μM)+57 μL 0.4% DMSO HBSS+60 μL acetonitrile (ACN) with internal standard (IS, 200 ng/mL Osalmid). The assay was performed as described below

### Routine Culture and Maintenance

Stock cultures are maintained in MEM+10% FBS+1% NEAA, grown in 75 cm² tissue culture treated flasks and split (passed) 2 times weekly to maintain desired confluence. For maintenance passage, trypsinized cells are routinely distributed into new flasks at a standard passage ratio of 1:4.

### Seeding Assay Plates

Caco-2 assay plates are seeded 21-27 days prior to running the assay. 24-Well plates are seeded at a cell density of 0.17×105/well in a 250 μL apical chamber volume (6.6×104/mL) with a 1 mL volume of growth medium to the 24-well basal chamber. Assay plates are generally provided with a growth medium change every other day.

### Preparation of Assay Plates

After the desired cell growth period, the cell culture plate is removed from the incubator to allow the culture to equilibrate to room temperature (approximately 0.5 hour). The monolayer is washed exchanging the volume one time using sterile HBSS buffer, pH 7.4. The electrical resistance across the monolayer is measured using the Millicell ERS system ohm meter (The cells are to be used if (transepithelial electrical resistance) TEER is higher than 250 ohm*cm²).

### Preparation of the Cell Plates

The buffer is removed from the apical side and basolateral side. 600 μL of donor solution (for A-to-B) or 500 μL of receiver solution (for B-to-A) is added to the apical wells based on plate map. A fresh basolateral plate is prepared by adding 800 μL of receiver solution (for A-to-B) or 900 μL of donor solution (B-to-A) to the well of a new 24-well plate. The apical and the basolateral plates are incubated at 37 degrees C.

### Preparation of Analytical Plate

After 5 min, 100 μL of sample from all donors (for both A-to-B and B-to-A) is transferred into appropriate wells of a sample plate for D0. 100 μL of sample from all apical chambers (the donor of A-to-B and receiver of B-to-A) is transferred into appropriate wells of a microplate for Lucifer Yellow D0 (D0 LY). The apical plate is laid on the basolateral plate to start transport process. At 90 min, the apical and basolateral plates are separated and 100 μL of sample from all donors (for both A-to-B and B-to-A) transferred into appropriate wells of a new sample plate for D90, and 200 μL of sample from all receivers is transferred into appropriate wells of a sample plate for R90. Transfer 100 μL of samples from all basolateral chambers (receiver of A-to-B and donor of B-to-A) into appropriate wells of a new microplate for Lucifer Yellow R90 (R90 LY). Determine LY permeability by reading D0 LY and R90 LY at an excitation wavelength of 485 nm and an emission wavelength of 535 nm using a fluorescent plate reader.

### Sample Preparation

For receiver solution, samples are prepared using 60 μL of sample+60 μL ACN with IS (200 ng/mL Osalmid). For donor solution, samples are prepared using 6 μL of sample+54 μL 0.4% DMSO/HBSS+60 μL ACN with IS (200 ng/mL Osalmid).

### 7. Liver Microsome Stability Testing

Metabolic stability assays are designed to measure the stability of a test compound in a variety of assay matrices from human and animal species. From a metabolism perspective, a drug would be relatively stable, have a small first-pass effect, and maintain an effective concentration in blood for a reasonable period of time. A microsomal preparation from the liver contains all CYP isozymes and other membrane-bound drug metabolizing enzymes which are responsible for the metabolism for the majority of drugs in humans. Metabolic stability in liver microsomes can determine half-life (T1/2) and intrinsic clearance (Clint). The determination of Clint may be useful to determine whether metabolism is the major elimination pathway when it is compared to total body clearance in vivo. For high metabolic stability in liver microsomes: T1/2>120 min, for moderate metabolic stability in liver microsomes: 30 min<T1/2<120 min, and for low metabolic stability in liver microsomes: T1/2<30 min In this assay, test compounds are tested against human, Sprague Dawley rat and CD-1 mouse liver microsomes. Results are reported as % of parent compound remaining after 15, 30 and 60 minutes. Human, Sprague Dawley rat, and CD-1 mouse liver microsome samples were purchased from BD Gentest. An in vitro metabolic stability assay suitable for use in predicting first pass metabolism of the compounds useful in the methods of the invention is described in this example. The embodiments of this assay described in this example were developed by Shanghai ChemPartner Co., Ltd., a corporation of China with its principal office located at 720 Cailun Road, Building No. 3, Shanghai 201203, China.

### Reagents

Compound stock solutions (10 mM test compound in DMSO, stored at −80° C.), Assay Buffer (0.1 M Potassium phosphate buffer, pH 7.4), Buffer A (1.0 L of 0.1 M monobasic Potassium Phosphate buffer containing 1.0 mM EDTA), Buffer B (1.0 L of 0.1 M Dibasic Potassium Phosphate buffer containing 1.0 mM EDTA), Buffer C (K-phosphate buffer, 0.1 M Potassium Phosphate buffer, 1.0 mM EDTA, pH 7.4 by titrating 700 mL of buffer B with buffer A while monitoring the pH meter) are used.

### Spiking Solutions

Test compound or positive control solutions (3×) are prepared. 500 μM spiking solution is prepared by adding 10 μL of 10 mM DMSO stock solution into 190 μL ACN. For 1.5 μM spiking solution in microsomes (0.75 mg/mL), 1.5 μL of 500 μM spiking solution and 18.75 μL of 20 mg/mL liver microsomes are added into 479.75 μL of K-phosphate buffer.

### Other Solutions

6 mM NADPH in 0.1 M K-phosphate buffer is prepared by dissolving 25.1 mg of NADPH tetrasodium salt in 5 mL of K-phosphate buffer.

### Procedure

30 μL of 1.5 μM spiking solution containing 0.75 mg/mL microsomes solution is added to the wells designated for 60 min, 30 min, 15 min, 5 min and 0 min. The plate is pre-incubated at 37° C. for 10 minutes. 15 μL of NADPH stock solution (6 mM) is added to the wells designated for Time 60 and timed. At 30 min, 15 min, and 5 min, 15 μL of NADPH stock solution (6 mM) is added to the wells. At the end of incubation, 135 μL of ACN containing IS is added to all the wells (60 min, 30 min, 15 min, 5 min and 0 min). Then 15 μL of NADPH stock solution (6 mM) is added to the wells designated as Time 0. After quenching, the reaction mixtures is centrifuged at 3220×g for 10 min. 50 μL of the supernatant from each well is transferred into a 96-well sample plate containing 50 μL of ultra pure water (Millipore) for LC/MS analysis.

### 8. CYP Inhibition Testing

Metabolic drug-drug interactions may occur when a drug inhibits or induces the activity of a drug metabolizing enzyme such as a CYP, which may affect the metabolism of a concomitant drug. As a result, plasma concentrations of these drugs may increase, leading to potential toxicities.

Select compounds are tested for CYP inhibition against five different CYP enzymes: CYP 1A2, CYP 2C9, CYP 2C19, CYP 2D6, CYP 3A4. Individual CYP enzymes are incubated with corresponding substrates with no inhibitor (negative control). Individual CYP enzymes are then incubated with corresponding substrates in the presence of test compound. Results are reported as “CYP (% negative controls) 1A2=v %; 2C9=w %; 2C19=x %; 2D6=y %; 3A4=z %” where v %, w %, x %, y %, and z % are % inhibition with respect to the negative control. Values of v, w, x, y, z>75 indicate low level of inhibition of the corresponding enzyme. Values of 70>v, w, x, y, z>30 indicate moderate level of inhibition of the corresponding enzyme.

The following reagents are used: Sodium Phosphate, monobasic; Sodium Phosphate, dibasic; β-Nicotinamide Adenine Dinucleotide Phosphate-Reduced (NADPH), Roche; Milli-Q Water; 0.1 M Potassium phosphate buffer (K-buffer), pH 7.4. The following stock solutions are used. Stock A (136.5 g of monobasic potassium phosphate in 1 L of Milli-Q water (1.0 M)). Stock B (174.2 g of dibasic potassium phosphate in 1 L of Milli-Q water (1.0 M)). 40.5 mL of Stock B is mixed with 9.5 mL of Stock A, and the total volume brought near 500 mL with Milli-Q water to give 0.1 M potassium phosphate buffer. The buffer is titrated with KOH or H₃PO₄ to pH 7.4. To prepare a cofactor buffer (4×, 8 mM NADPH), 66.7 mg of NADPH tetrasodium salt is dissolved in 10 mL of K-buffer. For human liver microsome solution, a stock solution (20 mg/mL (4×final)) is used.

The following Positive control stock solutions are prepared: 0.3 mM α-Naphthoflavone (MW: 272.3, 0.0817 mg of α-Naphthoflavone in 1 mL DMSO), 10 mM Sulfaphenazole (MW: 314.4, 3.14 mg of sulfaphenazole in 1 mL DMSO) 100 mM Omeprazole (MW: 345.4, 34.54 mg of omeprazole in 1 mL DMSO), 2.5 mM Quinidine (MW: 324, 0.81 mg of quinidine in 1 mL DMSO), and 2.5 mM Ketoconazole (MW: 531.4, 1.33 mg of ketoconazole in 1 mL DMSO).

The following substrate stock solutions are prepared: 6 mM Phenacetin (MW: 179.22, 1.075 mg of phenacetin in 1 mL can), 10 mM Diclofenac (MW: 318.83, 3.18 mg of Diclofenac in 1 ml H₂O), 35 mM S-Mephenyloin (MW: 218.25, 7.64 mg of s-mephenyloin in 1 mL ACN 10 mM Bufuralol (MW: 297.82, 2.98 mg of Bufuralol in 1 ml H₂O), 1 mM Midazolam (MW: 325.77, 0.326 mg of midazolam in 1 mL can), and 10 mM Testosterone (MW: 288.42, 2.88 mg of Testosterone in 1 ml ACN.

### Assay Procedure

Test compounds and reference compounds (positive control) are prepared in 0.2 mg/ml liver microsome solution. A 0.2 mg/ml liver microsome solution (2×final) is prepared from stock solution by adding 10 μL microsome stock solutions (20 mg/ml) in 990 μL K-buffer. 8 μL of 10 mM test compound stock solution (in DMSO or other solvent at various concentrations) is diluted with 12 μL ACN to make 4 mM solutions. 1:3 serial dilutions are performed using DMSO/ACN (40:60) from 4 mM solution further down seven concentration points (400×final): 4 mM, 1.33 mM, 0.44 mM, 0.148 mM, 0.1494 mM, 0.0165 mM, 0.00549 mM, 0 mM. 8 μL of reference compound stock solution in DMSO is diluted with 12 μL ACN. 1:3 serial dilutions are performed using DMSO/ACN (40:60) from diluted concentration further down seven concentration points (400×final). 2 μL of serially diluted test compounds are added to 400 μL of 0.2 mg/mL microsome solution. 1 μL of serially diluted reference compound is added to 200 μL of 0.2 mg/mL microsome solution.

The substrate solution (4×final) is prepared for CYP2C19 in microsome solution. substrate solutions (4×final) for other CYPs is prepared in K-buffer. The following pre-warmed solutions (in duplicates) are added in a 96-well assay plate: 30 μL of 2×test compound and reference compound in 0.2 mg/mL microsome solution. Add 15 μL of 4×substrate solutions (CYP2C19 contains liver microsomes). Samples are pre-incubated for 10 minutes at 37° C. 15 μL pre-warmed 4×NADPH cofactor solution (see step 6.7) is added into the assay plate to initiate the reaction and incubated (37° C.) 5 minutes for CYP3A4, 10 minutes for CYP1A2, CYP2C9 and CYP2D6, and 45 minutes for CYP2C19. Stop the reaction by adding 120 μL ACN containing IS. Centrifuge the assay plate (4000 rpm) for 15 minutes and transfer the supernatants into a new 96-well plate for LC/MS analysis. Curve-fit to calculate IC₅₀ using a Sigmoidal (non-linear) dose-response model (GraphPad Prism 5.0) based on data calculation using the formula Y=Bottom+(Top-Bottom)/(1+10̂((Log EC50−X)*HillSlope)) where X is the logarithm of concentration, and Y is the response starting from Bottom to Top in a sigmoid shape in response to inhibitor concentration from high to low.

While certain embodiments have been illustrated and described, it will be understood that changes and modifications can be made therein in accordance with ordinary skill in the art without departing from the present invention in its broader aspects as defined in the following claims.

### 9. In Vivo Rat Pharmacokinetic Studies

In vivo rat pharmacokinetic studies are designed to measure bioavailability, tissue distribution and metabolite identification in rats after intravenous (IV) and oral (PO) dosing. A full PK study typically includes two study arms (IV and PO) and takes serial blood samples from multiple animals per compound. The assay as described in this example was developed by BioDuro, a corporation of China with its principal office located at Building E, No. 29 Life Science Park Road, Changping District, Beijing, 102206, P.R. China.

TABLE 3

| | # of | Dosing | Dose | |
|---|---|---|---|---|
| Group | rats | Route | (mpk) | Timepoints (h) |
| 1 | 3 | IV | 1 | Plasma draw: 0, 0.167, 0.833, 0.25, |
| | | | | 0.5, 1, 2, 4, 8, 12, 24 |
| 2 | 3 | PO | 20 or 40 | Plasma draw: 0, 0.25; Liver draw: |
| | | | | 0.25 |
| 3 | 3 | PO | 20 or 40 | Plasma draw: 0.5, 4; Liver draw: 4.0 |
| 4 | 3 | PO | 20 or 40 | Plasma draw: 0, 1; Liver draw: 1 |
| 5 | 3 | PO | 20 or 40 | Plasma draw: 2, 8; Liver draw: 8 |
| 6 | 3 | PO | 20 or 40 | Plasma draw: 12, 24; Liver draw: 24 |

EBP1047, EBP1595, EBP1597, and EBP1604 were tested in rats, in vivo, to demonstrate their bioavailability and liver concentration. EBP1047, EBP1595, EBP1597, and EBP1604 demonstrate HCV 1b replicon activity between 0.5-2 μM, in vitro PK profiles, which predict higher stability in human liver vs rat liver, and moderate intestinal absorption.

TABLE 4

| | | EBP1047 | EBP1595 | EBP1597 | EBP1604 |
|---|---|---|---|---|---|
| 1b replicon | | 0.5 μM | 1 μM | 0.9 μM | 2.2 μM |
| LM | human | 78/62/34 | 73/46/14 | 81/74/57 | 71/63/28 |
| (% parent | rat | 44/18/10 | 67/46/8 | 57/24/15 | 58/41/14 |
| remaining at | mouse | 56/29/12 | 67/50/17 | 67/32/14 | 57/40/14 |
| 15, 30, 60 min) | | | | | |

| | | | | |
|---|---|---|---|---|
| Efflux/Permeability | 8/11 = 0.8 | 14/15 = | 7/9 = 0.8 | 9/12 = 0.8 |
| (desired <3) | | 0.9 | | |

Each compound was singly dosed both IV (1 mpk (mg/kg) to rats 1-3) and PO (20 or 40 mpk to rats 4-18), followed by blood draws and liver concentration studies (for PO dosed rats) from each animal at each time point. All four compounds exhibit higher liver concentrations than in plasma, with liver/plasma ratios (L/P ratio)>170 at Cmax.

Concentrations of EBP1047 in liver (ng/g) indicate that between 1 h and 8 h post dosing, liver concentrations are in the range of 1-30 μM. With an EC₅₀ of 0.5 μM for EBP1047, for once a day (qd) dosing, the concentration of EBP1047 in liver can be between 2-60 fold above the EC₅₀, for at least 8 hours. Thus, this compound and compounds with similar activity profiles can be dosed once, twice, or thrice (or more frequently) daily to treat HCV infection.

Concentrations of EBP1595 in liver (ng/g) indicate that between 0.25 h and 4 h post dosing, liver concentrations are in the range of 1-32 μM. With an EC₅₀ of 1 μM for EBP1595, for qd dosing, the concentration of EBP1595 in liver can be between 2-32 fold above the EC₅₀, for at least 4 hours. Thus, this compound and compounds with similar activity profiles can be dosed twice, or thrice (or more frequently) daily to treat HCV infection.

Concentrations of EBP1597 in liver (ng/g) indicate that between 0.25 h and 12 h post dosing, liver concentrations are in the range of 2-18 μM. With an EC₅₀ of 0.9 μM for EBP1597, for qd dosing, the concentration of EBP1597 in liver may be between 2-18 fold above the EC₅₀, for at least 12 hours. During these 12 hours, the concentration of EBP1597 in liver is higher than its EC₅₀. Thus, this compound and compounds with similar activity profiles can be dosed once, twice, or thrice (or more frequently) daily to treat HCV infection.

Concentrations of EBP1604 in liver (ng/g) indicate that between 0.25 h and 1 h post dosing, liver concentrations are in the range of 2-18 μM. With an EC₅₀ of 2.2 μM, for qd dosing, the concentration of EBP1604 may be up to 6 fold above its EC₅₀, for at least 1 hour. Thus, this compound and compounds with similar activity profiles can be dosed twice or thrice (or more frequently) daily to treat HCV infection.

These results are tabulated below. BID, TID, or more frequent dosing can further extend the exposure of these compounds in the liver at concentrations above the EC₅₀ concentrations of these compounds. For treatments where longer liver exposure to a drug is desired, EBP1047, EBP1595, or EBP1597 can provide the required exposure. For treatments where shorter liver exposure to a drug is sufficient, EBP1047, EBP1595, EBP1597, and EBP1604 can provide the required exposure.

TABLE 5

| | EBP1047 | | | EBP1595 | | | EBP1597 | | | EBP1604 | | |
|---|---|---|---|---|---|---|---|---|---|---|---|---|
| | 20 mpk | | | 20 mpk | | | 40 mpk | | | 20 mpk | | |
| Time | Plasma | Liver | L/P | Plasma | Liver | L/P | Plasma | Liver | L/P | Plasma | Liver | L/P |
| (h) | (ng/mL) | (ng/g) | Ratio | (ng/mL) | (ng/g) | Ratio | (ng/mL) | (ng/g) | Ratio | (ng/mL) | (ng/g) | Ratio |
| 0.25 | 5.1 | 382 | 78.3 | 1.6 | 1900 | 652 | 1.6 | 2625 | 1673 | 0.8 | 915 | 618 |
| 1 | 24.1 | 14010 | 558 | 96.4 | 12450 | 172 | | | | 17.9 | 4868 | 275 |
| 4 | 10.4 | 2668 | 261 | 14.0 | 1398 | 67 | 10.3 | 8118 | 717 | 0 | 203 | NA |
| 8 | 2.9 | 563 | 182 | 0 | 132 | NA | 2.6 | 1498 | 582 | 0 | 43 | NA |
| 12 | | | | | | | 2.5 | 1030 | 527 | | | |
| 24 | 0 | 5.35 | NA | 0 | 0 | NA | | | | 0 | 0 | NA |

| | | | | |
|---|---|---|---|---|
| Liver | 1-30 μM (8 h) | 4-32 μM (4 h) | 2-18 μM (12 h) | 2-12 μM (1 h) |
| Conc | EC₅₀ = 0.5 μM | EC₅₀ = 1 μM | EC₅₀ = 0.9 μM | EC₅₀ = 2.2 μM |
| % F | 9 | 9 | 9 | 2 |

In general, these results demonstrate that compounds with R₁=small aliphatic (containing less than 3 carbons, e.g., alkyl groups); R₅=sterically hindered amines (7-8 membered ring, N,N-methyl-isobutyl, or 2-alkyl substituted piperidine); R₂₂=small aliphatic group (not containing greater than 5 carbons, e.g., alkyl and cycloalkyl groups) or R₂₂═—CH₂CH₂-secondary or -tertiary amine (containing no more than 7 carbons) can have good oral absorption and therefore be useful in anti viral treatment, for example, as orally administered compounds.

While this invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1.-31. (canceled)

32. A compound of formula: 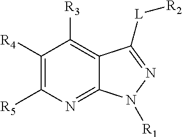
or a pharmaceutically acceptable salt thereof, wherein
R₁ is hydrogen; C₁-C₆ alkyl; C₁-C₆ alkyl substituted with a substituted or unsubstituted C₃-C₈ cycloalkyl, 5-8 membered heterocyclyl, a heteroaryl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl-, —CO-heterocyclo-, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;
L is a bond, —CONH—, —NH—CO—, substituted or unsubstituted C₁-C₅ alkylene, substituted or unsubstituted C₂-C₅ heteroalkylene, a substituted or unsubstituted 5 membered heteroaryl group, or a combination thereof;
R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group;
R₃ and R₄ are hydrogen;
R₅ is —OH, —OR′, —NHR′, —NR′R′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and —N(R′)C(O)NHR′; and
R′ is a substituted or unsubstituted C₃-C₈ cycloalkyl, aryl, heteroaryl, or heterocyclyl group, or two R′ groups together with the nitrogen atom to which they are bonded form a heterocyclic ring.

33. The compound of claim 32 of formula: 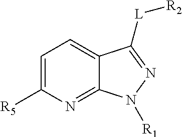
wherein
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO—(C₃-C₈ cycloheteroalkyl), —CO—(C₁-C₆ heteroalkyl), —SO₂—(C₁-C₆ cycloalkyl), or —SO₂—(C₃-C₈ cycloalkyl) group;
L is a bond, —CONH—, —NH—CO—, substituted or unsubstituted C₁-C₅ alkylene, substituted or unsubstituted C₂-C₅ heteroalkylene, or a combination thereof;
R₂ is a substituted or unsubstituted 5-7 membered heterocyclyl, C₅-C₇ cycloalkyl, 5-6 membered heteroaryl, or a 6 membered aryl group;
R₅ is R₅₁R₅₂N—, R₅₃(MeSO₂)N—, R₅₄O—, or substituted or unsubstituted C₁-C₆ alkyl;
R₅₁ is hydrogen or C₁-C₃ alkyl;
R₅₂ is C₁-C₃ alkyl, substituted or unsubstituted cycloalkyl, aryl, heterocyclyl, or heteroaryl group, wherein each cycloalkyl, aryl, heterocyclyl, or heteroaryl group contains 6-8 ring atoms, or R₅₁ and R₅₂ together with the nitrogen atom to which they are bonded form a 6, 7, 8, or 9-membered heterocyclyl ring containing up to 3 heteroatoms substituted by a substituted or unsubstituted benzyl, acyl, or sulfonyl group;
R₅₃ is substituted and unsubstituted C₁-C₆ alkyl; and
R₅₄ is hydrogen, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups.

34. The compound of claim 33, wherein
R₁ is hydrogen, C₁-C₅ alkyl, or —(CH₂)ₖ—R₁₁;
k is 1 or 2; and
R₁₁ is C₃-C₈ cycloalkyl or a substituted or unsubstituted aryl or heteroaryl group;
L is —CONH— and the carbon atom of the —CO—NH— is bonded to the azaindazole ring, or L is —(CH₂)ₙ—, —O—(CH₂)ₙ—, or —CH₂—O—(CH₂)ₙ—; where the left hand side of the L is bonded to the azaindazole moiety; and n is 3, or 4.
R₂ is a substituted or unsubstituted 5-7 membered heterocyclyl; and
R₅ is —NR₅₁R₅₂, R₅₁ is H, methyl, or ethyl and R₅₂ is ethyl, isobutyl, cyclohexyl, cycloheptyl, cyclooctyl, or —NR₅₁R₅₂ is: 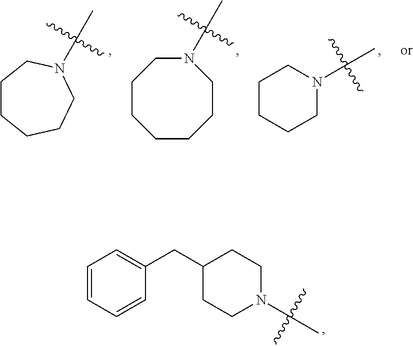
or
R₅ is:

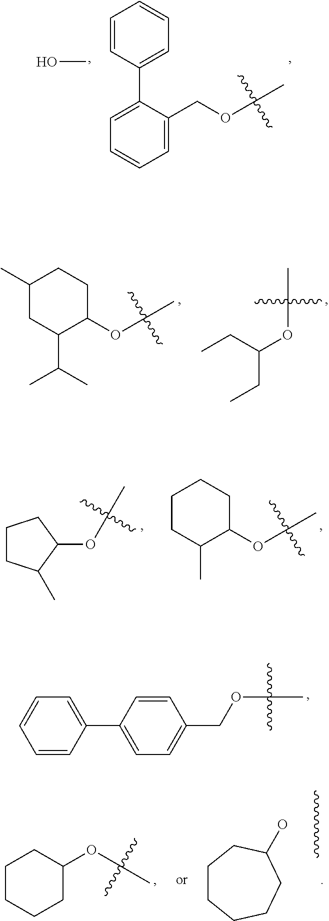

35. The compound of claim 33, wherein L is a substituted or unsubstituted C₁-C₅ alkylene or C₁-C₅ heteroalkylene group.

36. The compound of claim 33, wherein
L is —(CH₂)ₙ—, —O—(CH₂)ₙ—, or —CH₂—O—(CH₂)ₙ—;
the left hand side of the L is bonded to the azaindazole moiety; and
n is 1, 2, 3, or 4.

37. The compound of claim 33, wherein R₂ is substituted or unsubstituted piperidinyl, pyrrolidinyl, piperazinyl, or azepanyl group.

38. The compound of claim 33, wherein R₂ is 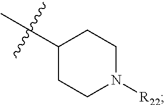
R₂₂ is C₂-C₃ alkyl or —CH₂CH₂—NR₂₃R₂₄; and
R₂₃ and R₂₄ are independently hydrogen, C₁-C₃ alkyl, C₁-C₃ alkyl substituted with a C₃-C₄ cycloalkyl ring, a 6-7 membered heterocycle, or R₂₃ and R₂₄ together with the nitrogen atom to which they are bonded form a substituted or unsubstituted 5-8 membered heterocyclic ring, wherein R₂ is —NR₂₃R₂₄ and R₂₃ and R₂₄ are independently hydrogen, C₁-C₃ alkyl, C₁-C₃ alkyl substituted with a C₃-C₄ cycloalkyl ring, a 6-7 membered heterocycle, or R₂₃ and R₂₄ together with the nitrogen atom to which they are bonded form a substituted or unsubstituted 5-8 membered heterocyclic ring.

39. The compound of claim 38, wherein NR₂₃R₂₄ is:

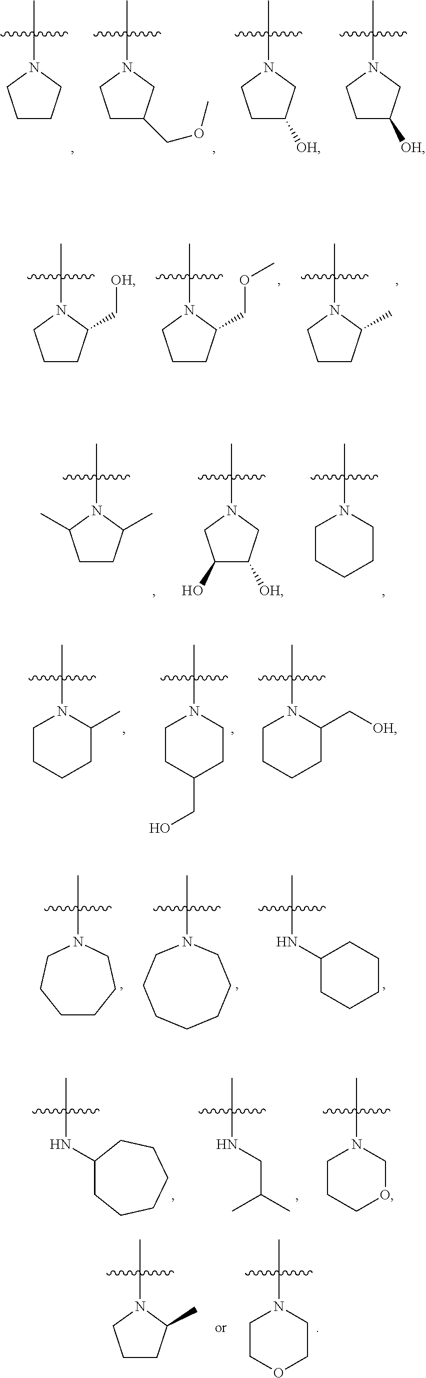

40. The compound of claim 33, wherein R₅ is —NR₅₁R₅₂, R₅₁ is H, methyl, or ethyl and R₅₂ is ethyl, isobutyl, cyclohexyl, cycloheptyl, cyclooctyl, or cyclohexylmethyl, or —NR₅₁R₅₂ is:

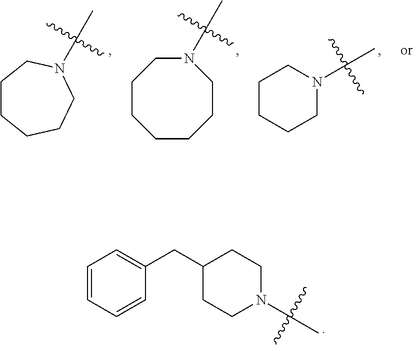

41. A compound that is 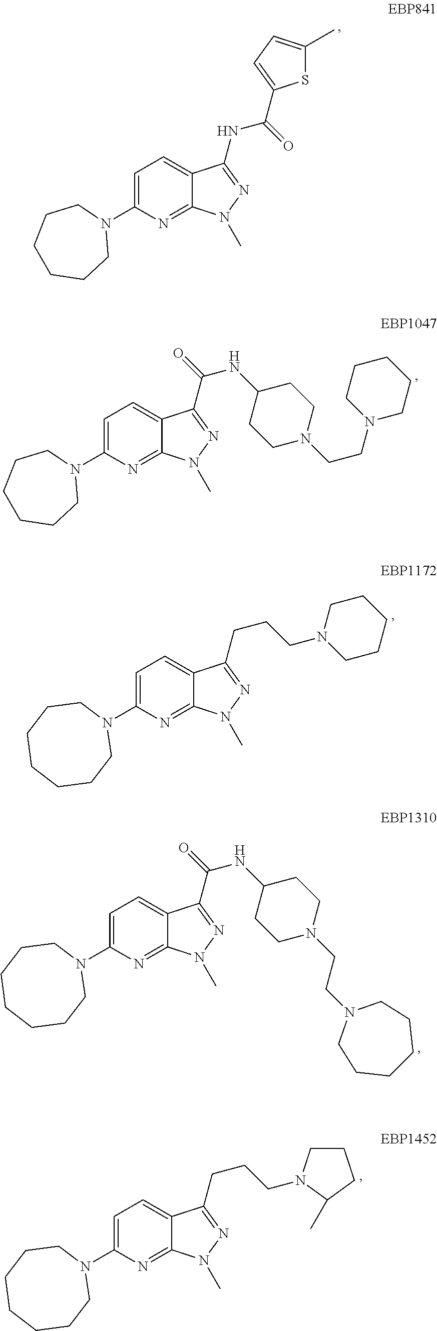 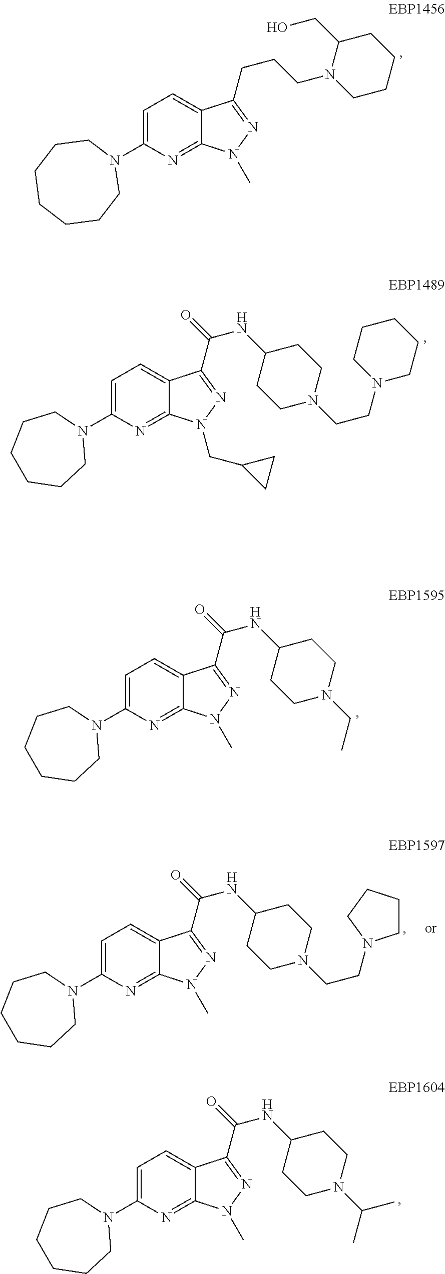
or a pharmaceutically acceptable salt thereof.

42. The composition of claim 33, further comprising a pharmaceutically acceptable carrier, excipient, or diluent.

43. The composition of claim 34, further comprising a pharmaceutically acceptable carrier, excipient, or diluent.

44. The composition of claim 35, further comprising a pharmaceutically acceptable carrier, excipient, or diluent.

45. The composition of claim 36, further comprising a pharmaceutically acceptable carrier, excipient, or diluent.

46. The composition of claim 37, further comprising a pharmaceutically acceptable carrier, excipient, or diluent.

47. The composition of claim 38, further comprising a pharmaceutically acceptable carrier, excipient, or diluent.

48. The composition of claim 39, further comprising a pharmaceutically acceptable carrier, excipient, or diluent.

49. The composition of claim 40, further comprising a pharmaceutically acceptable carrier, excipient, or diluent.

50. The composition of claim 41, further comprising a pharmaceutically acceptable carrier, excipient, or diluent.

51. The composition of claim 41, wherein the composition is a medicament for the treatment of HCV infection.
